(19) United States
(12) Patent Application Publication
(10) Pub. No.: US 2011/0104191 A1
(43) Pub. Date: May 5, 2011

(54) MALVA MOSAIC VIRUS AND VIRUS-LIKE PARTICLES AND USES THEREOF

(75) Inventor: Denis Leclerc, Fossambault-sur-le Lac (CA)

(73) Assignee: Universite Laval, Quebec City,, QC (CA)

(21) Appl. No.: 12/677,772

(22) PCT Filed: Sep. 11, 2008
(86) PCT No.: PCT/CA2008/001603

§ 371 Date: Nov. 30, 2010

Related U.S. Application Data
(60) Provisional application No. 60/971,486, filed on Sep. 11, 2007.

(51) Int. Cl. A61K 39/12; C12N 7/00; C07K 14/005; A61P 37/02

(52) U.S. Cl. 424/186.1; 424/204.1; 435/235.1; 530/350

(57) ABSTRACT

The immunogenic properties of the potexvirus MaI va mosaic virus (MaMV) and virus-like particles (VLPs) comprising the coat proteins of MaMV are disclosed VLPs prepared from MaMV coat proteins, methods of preparing the VLPs, MaMV coat protein polypeptides and polynucleotides encoding the coat protein are taught. Further, immunogenic compositions comprising MaMV or a VLP comprising the MaMV coat protein alone or in combination with one or more antigens and the use of said compositions to vaccinate and/or induce an immune response in an animal are also taught.

### FIELD OF THE INVENTION

The present invention relates to the field of vaccine formulations and adjuvants and, in particular, to vaccines and adjuvants based on plant virus particles.

### BACKGROUND OF THE INVENTION

Among the numerous new approaches to vaccine development, virus-like-particles (VLPs) made of viral nucleocapsids have emerged as a promising strategy. To date, two VLP vaccines, hepatitis B virus (HBV) and Human Papilloma Virus (HPV), have been shown to function efficiently in humans (Fagan et al., 1987, J. Med. Virol., 21:49-56; Harper et al., 2004, Lancet, 364:1757-1765). VLPs made from the human papillomavirus (HPV) major capsid protein L1, for example, were shown to provide 100% protection in woman against development of cervical cancers (Ault, K. A., 2006, Obstet. Gynecol. Surv. 61:S26-S31; Harper et al., 2004, Lancet 364:1757-1765, see also International Patent Application PCT/US01/18701 (WO 02/04007)). Platforms such as the bacteriophage Qβ (Maurer et al., 2005, Eur. J. Immunol. 35:2031-2040), the hepatitis B virus VLPs made of the viral core protein (Mihailova et al., 2006, Vaccine 24:4369-4377; Pumpens et al., 2002, Intervirology 45:24-32), and parvovirus VLPs (Antonis et al., 2006, Vaccine 24:5481-5490; Ogasawara et al., 2006, In Vivo 20:319-324) have also shown capacity to carry epitopes and induce a strong antibody response. Similarly, U.S. Pat. No. 6,627,202, describes HBV core proteins comprising antigens crosslinked by HBV capsid-binding peptides for use as epitope delivery systems, including antigens targeted to or derived from various viruses and bacteria.

The use of VLPs from plant viruses as epitope presentation systems has been described. Plant viruses are comprised mainly of proteins that are highly immunogenic, and possess a complex, repetitive and crystalline organisation. In addition, they are phylogenetically distant from the animal immune system, which makes them good candidates for the development of vaccines. For example, cowpea mosaic virus (CPMV), Johnson grass mosaic virus (JGMV), tobacco mosaic virus (TMV), and alfalfa mosaic virus (AIMV) have been modified for the presentation of epitopes of interest (Canizares, M. C. et al., 2005, Immunol. Cell. Biol. 83:263-270; Brennan et al., 2001, Molec. Biol. 17:15-26; Saini and Vrati, 2003, J. Virol. 77:3487-3494). International Patent Application PCT/GB97/01065 (WO 97/39134) describes chimaeric virus-like particles that comprise a coat protein and a non-viral protein, which can be used, for example, for presentation of peptide epitopes. International Patent Application PCT/US01/07355 (WO 01/66778) describes a plant virus coat protein, and specifically a tobamovirus coat protein, fused via a linker at the N-terminus to a polypeptide of interest, which may include an epitope of a pathogenic microorganism. International Patent Application PCT/US01/20272 (WO 02/00169) describes vaccines comprising either potato virus Y coat protein or a truncated bean yellow mosaic virus coat protein fused to a foreign peptide, and specifically a Newcastle Disease Virus or human immunodeficiency virus (HIV) epitope. Also, U.S. Pat. No. 6,042,832 describes methods of administering fusions of polypeptides, such as pathogen epitopes, with alfalfa mosaic virus or ilarvirus capsid proteins to an animal in order to raise an immune response.

VLPs derived from the coat protein of papaya mosaic virus (PapMV) and their use as immunopotentiators has been described (International Patent Application No. PCT/CA03/00985 (WO 2004/004761) and U.S. patent application Ser. No. 11/556,678 (US2007/0166322)). Expression of the PapMV coat protein in E. coli leads to the self-assembly of VLPs composed of several hundred CP subunits organised in a repetitive and crystalline manner (Tremblay et al., 2006, FEBS J 273:14). Studies of the expression and purification of PapMV CP deletion constructs further indicate that self-assembly (or multimerization) of the CP subunits is important for function (Lecours et al., 2006, Protein Expression and Purification, 47:273-280). The ability of PapMV VLPs comprising epitopes from either gp100 or the influenza virus M1 protein have been shown to induce MHC class I cross-presentation of the epitopes leading to expansion of specific human T cells (Leclerc, D., et al., J. Virol, 2007, 81(3):1319-26; Epub. ahead of print Nov. 22, 2006). In addition, PapMV VLPs comprising epitopes derived from the hepatitis C virus E2 envelope protein were shown to induce an humoral response in mice toward the PapMV VLP as well as the E2 peptide (Denis et al., 2007, Virology, 363(1): 59-68).

The use of papaya mosaic virus VLPs fused to affinity peptides capable of binding resting spores of Plasmodiophora brassicae has also been described (Morin et al., 2007, J. Biotechnology, 128: 423-434). The VLPs were shown to be capable of binding P. brassacae spores with high avidity and were proposed as an alternative to antibodies for the detection of P. brassacae.

VLPs derived from Potato Virus X (PVX) carrying various antigenic determinants from HIV, HCV, EBV or the influenza virus have been described (European Patent Application No. 1 167 530). The ability of the PVX VLP carrying an HIV epitope to induce antibody production in mice via humoral and cell-mediated pathways is also described. Additional adjuvants were used in conjunction with the PVX VLP to potentiate this effect.

Hepatitis B core protein or parvovirus VLPs have been reported to induce a CTL response even when they do not carry genetic information (Ruedl et al., 2002, Eur. Immunol. 32; 818-825; Martinez et al., 2003, Virology, 305; 428-435) and can not actively replicate in the cells where they are invaginated. The cross-presentation of such VLPs carrying an epitope from lymophocytic choriomeningitis virus (LCMV) or chicken egg albumin by dendritic cells in vivo has also been described (Ruedl et al., 2002, ibid.; Morón, et al., 2003, J. Immunol. 171:2242-2250). The ability of a hepatitis B core protein VLP carrying an epitope from LCMV to prime a CTL response has also been described, however, this VLP was unable to induce the CTL response when administered alone and failed to mediate effective protection from viral challenge. An effective CTL response was induced only when the VLP was used in conjunction with anti-CD40 antibodies or CpG oligonucleotides (Storni, et al., 2002, J. Immunol. 168:2880-2886). An earlier report indicated that porcine parvovirus-like particles (PPMV) carrying a peptide from LCMV were able to protect mice against a lethal LCMV challenge (Sedlik, et al., 2000, J. Virol. 74:5769-5775).

A potexvirus, Malva veinal necrosis potexvirus (MVNV), was reported in 1990 (Brunt et al., (eds.) 1996 onwards. “Plant Viruses Online: Descriptions and Lists from the VIDE Database. Version: 20 Aug. 1996”) in Brazil as a virus infecting Malva parviflora on which local lesions and systemic veinal necrosis was detected.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide Malva mosaic virus and virus-like particles (VLPs) and uses thereof. In accordance with one aspect of the invention, there is provided an immunogenic composition comprising Malva mosaic virus (MaMV) or a virus-like particle (VLP) comprising MaMV coat protein and a pharmaceutically acceptable carrier.

In accordance with another aspect, there is provided a method of inducing an immune response in an animal comprising administering to said animal an effective amount of a immunogenic composition of the invention.

In accordance with another aspect of the invention, there is provided a method of vaccinating an animal against a disease, disorder or infection comprising administering to said animal an effective amount of immunogenic composition comprising Malva mosaic virus (MaMV) or a virus-like particle (VLP) comprising MaMV coat protein and one or more antigens.

In accordance with another aspect of the invention, there is provided a use of Malva mosaic virus (MaMV) or a virus-like particle (VLP) comprising MaMV coat protein in the preparation of an immunogenic composition.

In accordance with another aspect of the invention, there is provided a virus-like particle (VLP) comprising Mavia mosaic virus (MaMV) coat protein.

In accordance with another aspect of the invention, there is provided a method of preparing a virus-like particle (VLP), said method comprising the step of expressing a Malva mosaic virus (MaMV) coat protein in a host cell.

In accordance with another aspect of the invention, there is provided a fusion protein comprising a Malva mosaic virus (MaMV) coat protein fused to an antigen.

In accordance with another aspect, there is provided an isolated polynucleotide encoding a fusion protein comprising a Malva mosaic virus (MaMV) coat protein fused to an antigen.

In accordance with another aspect, there is provided a host cell genetically engineered with a polynucleotide encoding a fusion protein comprising a Malva mosaic virus (MaMV) coat protein fused to an antigen.

In accordance with another aspect, there is provided a use of a fusion protein comprising a Malva mosaic virus (MaMV) coat protein fused to an antigen, a polynucleotide encoding a fusion protein comprising a Malva mosaic virus (MaMV) coat protein fused to an antigen or a host cell genetically engineered with a polynucleotide encoding a fusion protein comprising a Malva mosaic virus (MaMV) coat protein fused to an antigen to prepare a virus-like particle.

### BRIEF DESCRIPTION OF THE DRAWINGS

These and other features of the invention will become more apparent in the following detailed description in which reference is made to the appended drawings.

FIG. 1 presents the genome sequence of the Malva mosaic virus (SEQ ID NO:1). The sequence is available under GenBank Accession No. DQ660333.

FIG. 2 presents the amino acid sequence of the coat protein of the Malva mosaic virus (SEQ ID NO:2).

FIG. 3 presents nucleotide sequence encoding the coat protein of the Malva mosaic virus (SEQ ID NO:3).

FIG. 4 depicts (A) a photograph showing mosaic symptoms on Malva spp. infected with Malva mosaic virus, (B) a photograph showing mosaic symptoms on Chenopodium quinoa when inoculated with an extract from Malva neglecta Wallr. infected leaves, (C) a photograph showing local lesions observed on C. quinoa when inoculated with an extract from C. quinoa infected leaves, and (D) an electron micrograph of purified Malva mosaic virus (bar represents 50 nm).

FIG. 5 depicts the results from an analysis of the 5′ end of Malva mosaic virus. The bars indicate the frequency that each sequence has been found in all the clones sequenced.

FIG. 6 (A) presents a schematic representation of the genomic organization of Malva mosaic virus (MaMV), (B) depicts a Northern Blot showing total RNA extracted from healthy (lane 1) or infected (lane 2) Chenopodium quinoa using a probe directed to MaMV coat protein, and (C) presents a sequence alignment of the octanucleotide putative sgPromoter sequence of various potexviruses (highly conserved regions are highlighted with black or grey boxes).

FIG. 7 presents a schematic representation of the secondary structure of the Malva mosaic virus 3′ untranslated region.

FIG. 8 presents the results of a phylogenetic analysis of replicase and capsid proteins of Malva mosaic virus and other potexviruses.

FIG. 9 (A) presents a SDS-PAGE gel showing purified recombinant Malva mosaic virus (MaMV) coat protein isolated from E. coli, and (B) depicts an electron micrograph of the MaMV VLP comprising the recombinant coat protein. Bar is 50 nm.

FIG. 10 presents (A) the nucleotide sequence of the MaMV coat protein gene contained in plasmid pMaMV-CP-6H (SEQ ID NO: 23) (the start codon is shown underlined and the stop codon is shown in bold and italicized); (B) the nucleotide sequence encoding the MaMV CP-SM protein (SEQ ID NO:62) (the sequence in italics and underlined indicates the two restriction enzyme recognition sequences); and (C) the nucleotide sequence encoding the MaMV CP gl-SM protein (SEQ ID NO:64) (the sequence in italics and underlined indicates the two restriction enzyme recognition sequences and the sequence encoding the spacer).

FIG. 11 presents the amino acid sequence of (A) the coat protein encoded by the MaMV coat protein gene contained in plasmid pMaMV-CP-6H (SEQ ID NO: 24); (B) MaMV CP-SM protein (SEQ ID NO:63) (the sequence in italics and underlined represents the amino acids inserted by inclusion of two restriction enzyme recognition sequences in the encoding nucleotide sequence); and (C) MaMV CP gl-SM protein (SEQ ID NO:65) (the sequence in italics and underlined represents the amino acids inserted by inclusion of two restriction enzyme recognition sequences in the encoding nucleotide sequence and the spacer sequence). The His-tag in each sequence is shown in bold.

FIG. 12 presents graphs demonstrating the production of: (A), (B) and (C), total IgG, IgG1 and IgG2a, respectively, in Balb/C mice injected s.c. once with MaMV VLPs in the absence of adjuvant as measured by ELISA; and (D), (E) and (F), total IgG, IgG1 and IgG2a, respectively, in the same mice injected s.c. a second time at day 40 with MaMV VLPs in the absence of adjuvant. ELISA was performed with blood collected at day 5, 10 and 14 after the first immunization and 4 days after the second immunization. + symbols in represent the negative control (pre-immune serum) that was used as the baseline for the ELISA.

FIG. 13 presents a SDS-PAGE gel showing the profile of the porins, OmpC and OmpF, purified from Salmonella typhi.

FIG. 14 depicts the HLA-A*0201 epitopes (in bold and underlined) from gp100 (SEQ ID NO:59) and influenza M1 protein (SEQ ID NO:60) together with their respective flanking sequences.

FIG. 15 presents A) an SDS-PAGE gel showing: first lane: molecular weight markers, second lane: bacterial lysate containing only the expression vector pET-3D without insert, third lane: lysate of E. coli cells expressing MaMV CP, and fourth lane: MaMV CP purified by affinity chromatography; B) an electron micrograph of MaMV virus-like particles comprising recombinant MaMV CP; C) elution profile of gel filtration chromatography showing that the purified MaMV CPs were of high molecular weight and were excluded from the column, suggesting that all the protein is in an oligomerization state of molecular weight exceeding 500 kDa.

FIG. 16 presents graphs demonstrating the production of: total IgG (A), IgG1 (B) and IgG2a (C) to Fluviral® and (D) IgG2a to the NP protein measured in the sera of Balb/C mice, 5 per group, 14 days after immunisation with Fluviral® adjuvanted either with MaMV VLPs (3 or 30 μg) or with alum.

FIG. 17 presents the nucleotide sequence encoding (A) the MaMV-M1 protein (SEQ ID NO:66) (the sequence encoding the M1 epitope is shown underlined); (B) the MaMV-gp100 protein (SEQ ID NO:68) (the sequence encoding the gp100 epitope is shown underlined); and (C) the MaMV gl-F3 protein (SEQ ID NO:70) (the sequence encoding the F3 peptide is shown underlined).

FIG. 18 presents the amino acid sequence of (A) the MaMV-M1 protein (SEQ ID NO:67) (the M1 epitope is shown in bold and underlined); (B) the MaMV-gp100 protein (SEQ ID NO:69) (the gp100 epitope is shown in bold and underlined); and (C) the MaMV gl-F3 protein (SEQ ID NO:71) (the F3 peptide is shown in bold and underlined). The His-tag in each sequence is shown in bold.

FIG. 19 presents A) an SDS-PAGE gel showing: first lane: molecular weight markers, second lane: bacterial lysate containing only the expression vector pET-3D without insert, third lane: lysate of E. coli cells expressing MaMV-SM protein, and fourth lane: MaMV-SM protein purified by affinity chromatography; B) an electron micrograph of MaMV virus-like particles comprising recombinant MaMV-SM protein; C) elution profile of gel filtration chromatography showing that the purified MaMV-SM protein was of high molecular weight and excluded from the column, suggesting that all the protein is in an oligomerization state of molecular weight exceeding 500 kDa.

FIG. 20 presents A) an SDS-PAGE gel showing: first lane: molecular weight markers, second lane: bacterial lysate containing only the expression vector pET-3D without insert, third lane: lysate of E. coli cells expressing MaMV gl-SM protein, and fourth lane: MaMV gl-SM protein purified by affinity chromatography; B) an electron micrograph of MaMV virus-like particles comprising recombinant MaMV gl-SM protein; C) elution profile of gel filtration chromatography showing that the purified MaMV gl-SM protein was of high molecular weight and excluded from the column, suggesting that all the protein is in an oligomerization state of molecular weight exceeding 500 kDa.

FIG. 21 presents A) an SDS-PAGE gel showing: first lane: molecular weight markers, second lane: bacterial lysate containing only the expression vector pET-3D without insert, third lane: lysate of E. coli cells expressing MaMV-M1 protein, and fourth lane: MaMV-M1 protein purified by affinity chromatography; B) an electron micrograph of MaMV virus-like particles comprising recombinant MaMV-M1 protein; C) elution profile of gel filtration chromatography showing that the purified MaMV-M1 protein was of high molecular weight and excluded from the column, suggesting that all the protein is in an oligomerization state of molecular weight exceeding 500 kDa.

FIG. 22 presents A) an SDS-PAGE gel showing: first lane: molecular weight markers, second lane: bacterial lysate containing only the expression vector pET-3D without insert, third lane: lysate of E. coli cells expressing MaMV-gp100 protein, and fourth lane: MaMV-gp100 protein purified by affinity chromatography; B) an electron micrograph of MaMV virus-like particles comprising recombinant MaMV-gp100 protein; C) elution profile of gel filtration chromatography showing that the purified MaMV-gp100 protein was of high molecular weight and excluded from the column, suggesting that all the protein is in an oligomerization state of molecular weight exceeding 500 kDa.

FIG. 23 presents A) an SDS-PAGE gel showing: first lane: molecular weight markers, second lane: bacterial lysate containing only the expression vector pET-3D without insert, third lane: lysate of E. coli cells expressing MaMV gl-F3 protein, and fourth lane: MaMV gl-F3 protein purified by affinity chromatography; B) an electron micrograph of MaMV virus-like particles comprising recombinant MaMV gl-F3 protein; C) elution profile of gel filtration chromatography showing that the purified MaMV gl-F3 protein was of high molecular weight and excluded from the column, suggesting that all the protein is in an oligomerization state of molecular weight exceeding 500 kDa.

FIG. 24 presents a graph showing the average length of different MaMV VLPs (100 VLPs from each recombinant construct were measured).

FIG. 25 presents flow cytometry results from pulsing CD40-activated B lymphocytes with fluorescently labelled Papaya mosaic virus (PapMV) VLPs, MaMV VLPs and MaMV-M1 VLPs.

FIG. 26 presents (A) flow cytometry results from pulsing T2 cells fluorescently labelled Papaya mosaic virus (PapMV) VLPs and MaMV VLPs showing the uptake of the VLPs over time; and (B) a graphical representation of the uptake of PapMV VLPs and MaMV VLPs by T2 cells over time as measured by mean fluorescence intensity (MFI).

FIG. 27 presents graphs showing (A) the amount of IFN-γ secreted by M1-specific T lymphocytes when added to T2 cells pulsed with PapMV VLPs or MaMV VLPs carrying the M1 (left) or gp100 (right) epitope or the corresponding M1 or gp100 peptide; (B) the amount of IFN-γ secreted by M1-specific T lymphocytes when added to T2 cells pulsed with different amounts of MaMV VLPs carrying the M1 (left) or gp100 (right) epitope or with the corresponding M1 or gp100 peptide, and (C) the amount of IFN-γ secreted by M1-specific T lymphocytes when added to T2 cells pulsed with PapMV VLPs or MaMV VLPs carrying the M1 (left) or gp100 (right) epitope or the corresponding M1 or gp100 peptide at 4° C. IFN-γ secretion was evaluated by ELISA.

FIG. 28 presents the results of in vitro T cell sensitization for donor #405 using PapMV VLPs or MaMV VLPs carrying an influenza M1 peptide: (A) shows the specificity of raised T cells evaluated by co-culture with the indicated pulsed T2 cells; and (B) presents the results of co-culturing the specific T cell lines with titrated concentrations of influenza M1 peptide or the gp100 peptide, as control.

FIG. 29 presents the results of in vitro T cell sensitization for donor #542 using PapMV VLPs or MaMV VLPs carrying an influenza M1 peptide: (A) shows the specificity of raised T cells evaluated by co-culture with the indicated pulsed T2 cells; and (B) presents the results of co-culturing the specific T cell lines with titrated concentrations of influenza M1 peptide or the gp100 peptide, as control.

FIG. 30 presents the results of in vitro T cell sensitization for donor #614 using PapMV VLPs or MaMV VLPs carrying an influenza M1 peptide: (A) shows the specificity of raised T cells evaluated by co-culture with the indicated pulsed T2 cells; and (B) presents the results of co-culturing the specific T cell lines with titrated concentrations of influenza M1 peptide or the gp100 peptide, as control.

FIG. 31 presents the results of in vitro T cell sensitization for donor #621 using PapMV VLPs or MaMV VLPs carrying an influenza M1 peptide: (A) shows the specificity of raised T cells evaluated by co-culture with the indicated pulsed T2 cells; and (B) presents the results of co-culturing the specific T cell lines with titrated concentrations of influenza M1 peptide or the gp100 peptide, as control.

FIG. 32 presents the results of an ELISA showing the total IgG response to (A) peptides F3, M1 or gp100, and (B) the MaMV CP, in mice injected with MaMV-SM VLPs, MaMV gl-F3 VLPs, MaMV gl-SM VLPs+F3, MaMV-M1 VLPs or MaMV-gp100 VLPs. 10 mice per group.

FIG. 33 presents (A) an alignment of the MaMV and PapMV CP amino acid sequences revealing a 31.2% identity between the two CPs; (B) the results of an ELISA revealing that antibodies directed to the MaMV CP are unable to recognise PapMV VLPs; and (C) the results of an ELISA revealing that antibodies directed to the MaMV CP are unable to recognise the PapMV VLPs.

### DETAILED DESCRIPTION OF THE INVENTION

The present invention relates to the immunogenic properties identified herein of the potexvirus Malva mosaic virus (MaMV) and virus-like particles comprising the coat protein of MaMV. The invention encompasses various embodiments related to the generation of an immune response in an animal that are based on these immunogenic properties of MaMV and MaMV VLPs. In one aspect, for example, the invention provides for the use of MaMV or VLPs comprising MaMV coat protein to prepare an immunogenic composition; for immunogenic compositions comprising MaMV or a virus-like particle comprising MaMV coat protein and optionally one or more antigens, and for the use of these compositions to induce an immune response in and/or to vaccinate an animal. Another aspect of the invention provides for the use of the MaMV coat protein to prepare VLPs; for VLPs prepared from MaMV coat protein; for methods of preparing such VLPs; coat protein polypeptides suitable for preparing the VLPs and polynucleotides encoding same.

In some embodiments, administration of MaMV or VLPs comprising MaMV coat protein alone is sufficient to raise an immune response and such administration can be used, for example, to “prime” the immune system prior to administration of an antigen. MaMV and MaMV VLPs can also be used as an adjuvant, in which context their immunogenic properties can be useful, for example, to enhance an immune response to an antigen. The antigen in this context can be administered prior to the MaMV or VLP, after the MaMV or VLP or concurrently with the MaMV or VLP. When administered concurrently, the antigen can be attached to the MaMV or VLP or can be separate. In one embodiment of the invention, the antigen is attached to the coat protein of the MaMV or MaMV VLP. MaMV and MaMV VLPs can also be used in the preparation of vaccines.

In accordance with one embodiment of the present invention, immunogenic compositions comprising the MaMV or MaMV VLP and one or more antigens are capable of inducing a humoral and/or cellular immune response in an animal.

### DEFINITIONS

Unless defined otherwise, all technical and scientific terms used herein have the same meaning as commonly understood by one of ordinary skill in the art to which this invention belongs.

As used herein, the term “about” refers to approximately a +/−10% variation from a given value. It is to be understood that such a variation is always included in any given value provided herein, whether or not it is specifically referred to.

The term “virus-like particle” (VLP), as used herein, refers to a self-assembling particle which has a similar physical appearance to a virus particle. The VLP may or may not comprise viral nucleic acids. VLPs are generally incapable of replication.

The term “pseudovirus,” as used herein, refers to a VLP that comprises nucleic acid sequences, such as DNA or RNA, including nucleic acids in plasmid form. Pseudoviruses are generally incapable of replication.

As used herein, a “fusion protein” is a protein that is created when two or more polynucleotides encoding two separate polypeptides, for example proteins or protein fragments, are genetically combined to provide a third polynucleotide encoding a protein (the “fusion protein”) that is a combination of the two polypeptides. For example, a polypeptide that comprises the amino acid sequence of the MaMV coat protein and the amino acid of an antigen would be considered to be a fusion protein.

The term “adjuvant,” as used herein, refers to an agent that augments, stimulates, actuates, potentiates and/or modulates an immune response in an animal.

The term “immunogenic,” as used herein, refers to the ability of a substance to induce a detectable immune response in an animal.

The term “immune response,” as used herein, refers to an alteration in the reactivity of the immune system of an animal in response to administration of a substance (for example, a compound, molecule, material or the like) and may involve antibody production, induction of cell-mediated immunity, complement activation, development of immunological tolerance, or a combination thereof.

The term “immunoprotective response,” as used herein, means an immune response that is directed against one or more antigen so as to protect against a condition (for example, a disease or disorder) and/or infection caused by an agent from which the one or more antigens are derived. For purposes of the present invention, immunoprotection against a condition and/or infection includes not only the absolute prevention of the condition or infection, but also any detectable reduction in the degree or rate of the condition or infection, or any detectable reduction in the severity of the condition or any symptom resulting from infection by the agent in a treated animal as compared to an untreated animal suffering from the condition or infection. An immunoprotective response can be induced in animals that were not previously suffering from the condition, have not previously been infected with the agent and/or do not have the condition or infection at the time of treatment. An immunoprotective response can also be induced in an animal already suffering from the condition or infected with the pathogen at the time of treatment. The immunoprotective response can be the result of one or more mechanisms, including humoral and/or cellular immunity.

The terms “immune stimulation” and “immunostimulation” as used interchangeably herein, refer to the ability of a molecule, such as a MaMV VLP, that is unrelated to an animal pathogen or disease to provide protection against infection by the pathogen or against the disease by stimulating the immune system and/or improving the capacity of the immune system to respond to the infection or disease. Immunostimulation may have a prophylactic effect, a therapeutic effect, or a combination thereof.

The term “vaccination,” as used herein, refers to the administration of a vaccine to a subject for the purposes of generating an immunoprotective response. Vaccination may have a prophylactic effect, a therapeutic effect, or a combination thereof. Vaccination can be accomplished using various methods depending on the subject to be treated including, but not limited to, parenteral administration, such as intraperitoneal injection (i.p.), intravenous injection (i.v.) or intramuscular injection (i.m.); oral administration; intranasal administration; intradermal administration, transdermal administration and immersion.

The term “vaccine,” as used herein, refers to a composition administered to a subject for the purpose of producing an immunoprotective response.

“Naturally-occurring,” as used herein, as applied to an object, refers to the fact that an object can be found in nature. For example, an organism (including a virus), or a polypeptide or polynucleotide sequence that is present in an organism that can be isolated from a source in nature and which has not been intentionally modified by man in the laboratory is naturally-occurring.

The terms “polypeptide” or “peptide” as used herein is intended to mean a molecule in which there is at least four amino acids linked by peptide bonds.

The expression “viral nucleic acid,” as used herein, refers to the genome or a portion thereof of a virus, or a nucleic acid molecule complementary in base sequence to that genome or portion. A DNA molecule that is complementary to viral RNA is also considered viral nucleic acid, as is a RNA molecule that is complementary in base sequence to viral DNA.

The terms “immunogen” and “antigen,” as used herein, refer to a molecule, molecules, a portion or portions of a molecule, or a combination of molecules, up to and including whole cells and tissues, which are capable of inducing an immune response in a subject alone or in combination with an adjuvant. The immunogen/antigen may comprise a single epitope or may comprise a plurality of epitopes. For example, peptides, polypeptides, proteins, glycoproteins, lipoproteins, carbohydrates, lipopolysaccharides, nucleic acids, small molecules, and various microorganisms, in whole or in part, including viruses, bacteria and parasites, and other infectious particles, may thus be antigens provided that they are capable of inducing an immune response. Haptens and mimotopes are also considered to be encompassed by the terms “immunogen” and “antigen” as used herein.

The term “prime” and grammatical variations thereof, as used herein, means to stimulate and/or actuate an immune response in an animal prior to administering an antigen.

As used herein, the terms “treat,” “treated,” or “treating” when used with respect to a condition, such as a disease or disorder, or infectious agent refers to a treatment which increases the resistance of a subject to the condition or to infection with a pathogen (i.e. decreases the likelihood that the subject will contract the condition or become infected with the agent) as well as a treatment after the subject has contracted the condition or become infected in order to fight a condition or infection (for example, reduce, eliminate, ameliorate or stabilise a condition or infection, or symptoms associated therewith).

The term “subject” or “patient” as used herein refers to an animal in need of treatment.

The term “animal,” as used herein, refers to both human and non-human animals. Non-human animals, include, but are not limited to, mammals, birds, reptiles and fish, and encompass domestic animals (including pets), farm animals, laboratory animals, zoo animals and wild animals, such as, for example, cows, pigs, horses, goats, sheep and other hoofed animals; dogs; cats; chickens, ducks and other birds; non-human primates; guinea pigs; rabbits; ferrets; rats; hamsters and mice.

The term “substantially identical,” as used herein in relation to a nucleic acid or amino acid sequence indicates that, when optimally aligned, for example using the methods described below, the nucleic acid or amino acid sequence shares at least 80%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length used in an alignment will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The terms “corresponding to” or “corresponds to” indicate that a nucleic acid sequence is identical to all or a portion of a reference nucleic acid sequence. In contradistinction, the term “complementary to” is used herein to indicate that the nucleic acid sequence is identical to all or a portion of the complementary strand of a reference nucleic acid sequence. For illustration, the nucleic acid sequence “TATAC” corresponds to a reference sequence “TATAC” and is complementary to a reference sequence “GTATA.”

### Immunogenic Compositions Comprising MaMV or MaMV Virus-Like Particles

The present invention provides for immunogenic compositions comprising MaMV or a MaMV VLP and optionally one or more antigens. The immunogenic compositions may further optionally comprise a suitable carrier, excipient or the like, and/or other standard components of pharmaceutical compositions that improve the stability, palatability, pharmacokinetics, bioavailability or the like, of the composition.

### Malva Mosaic Virus

A representative example of a MaMV suitable for use in the immunogenic compositions of the invention was isolated from Malva neglecta harvested in the vicinity of Summerland, British Columbia, Canada, and is described in Example 1. Analysis of the genome sequence and physical characteristics of the virus, as described in Examples 1 to 4 herein, indicate that MaMV is a member of the potexvirus genus, and most likely of the Flexiviridae family.

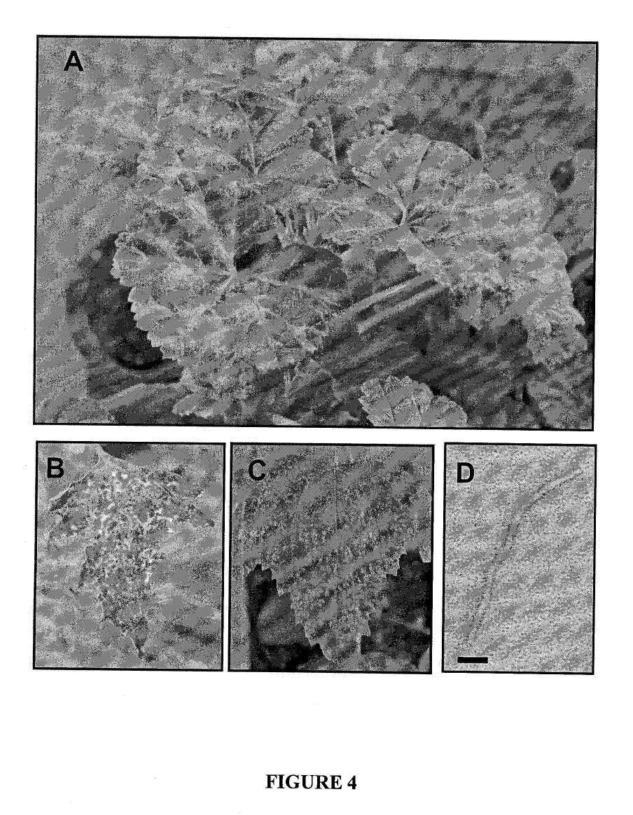

MaMV is characterised as being a flexible filamentous virion of between about 450 nm and about 600 nm in length and between about 10 nm and about 18 nm in width. The exemplary virus shown in FIG. 4D is between about 520 and 560 nm in length and between about 12 and about 16 nm in width.

Infection of M. neglecta with MaMV produces mosaic symptoms and vein clearing. MaMV is also characterised by its ability to propagate on members of the family Chenopodiaceae, for example, Chenopodium quinoa.

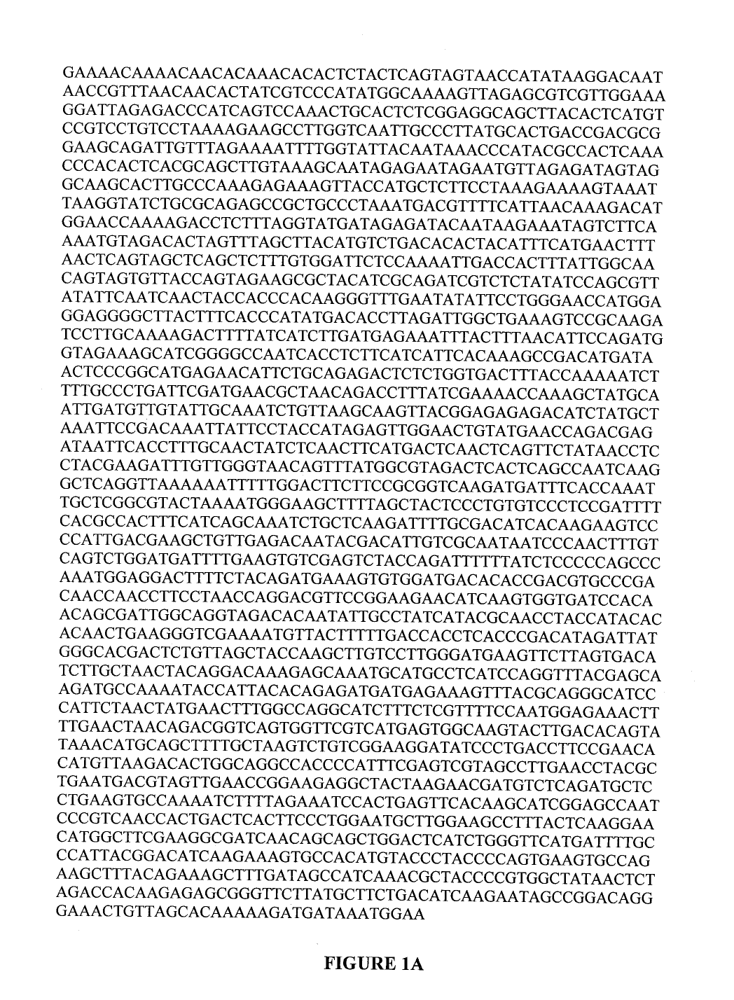

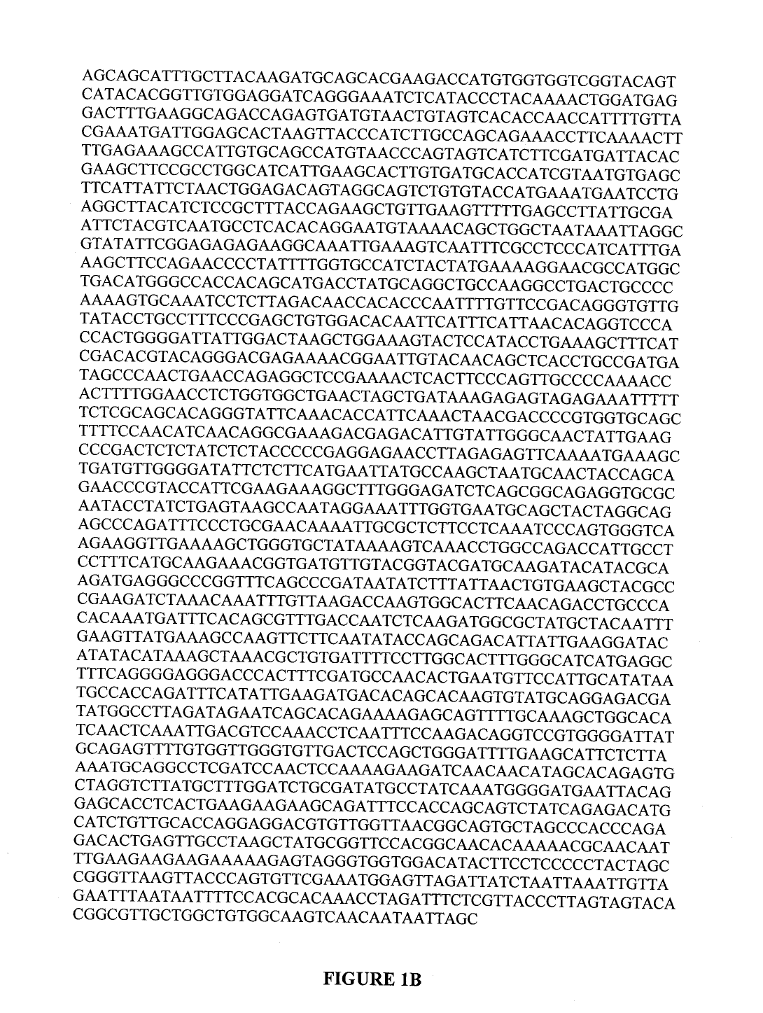

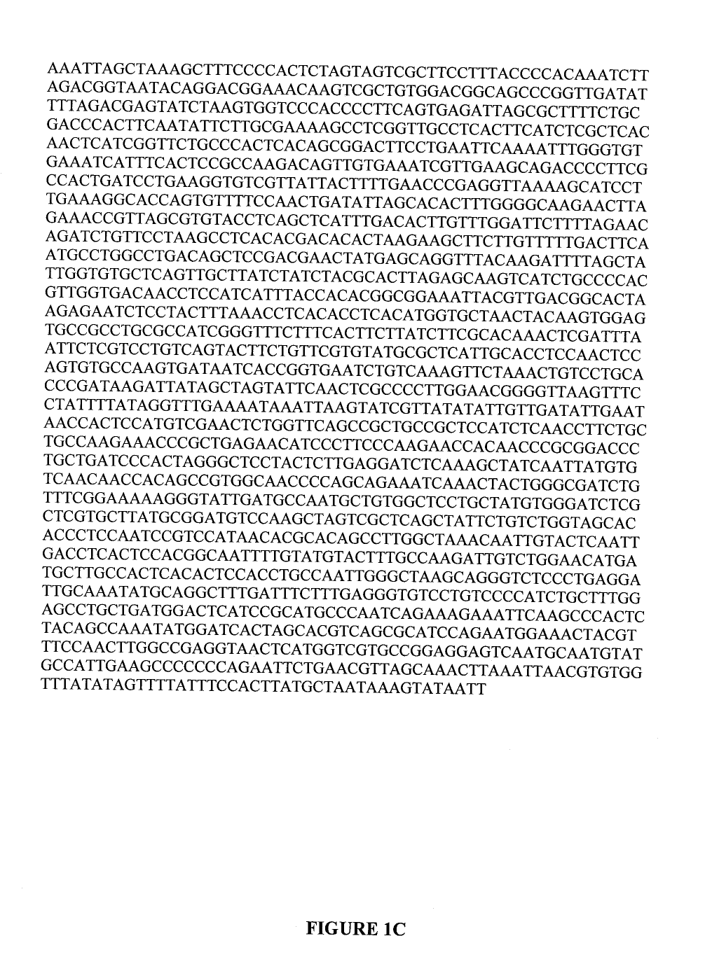

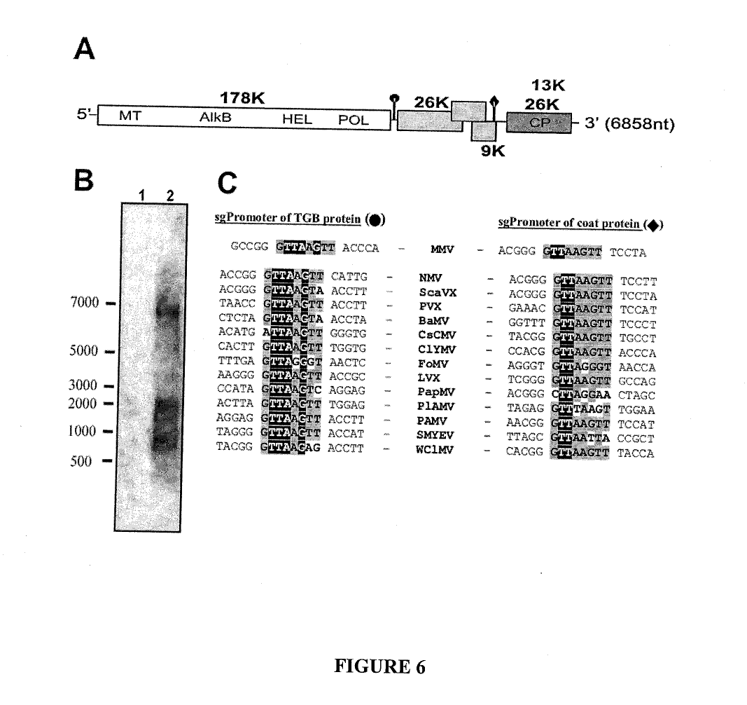

MaMV is further characterised as having a genome sequence substantially identical to the sequence as set forth in SEQ ID NO:1 (see also FIG. 1 and GenBank Accession No. DQ660333). MaMV genomic RNA is 6858 nucleotides (nt) long (excluding the poly(A) tail) with a GC content of 45%. The genomic organization is similar to other potexviruses, comprising a putative RNA-dependent RNA polymerase (RdRp), followed by three overlapping genes coding for the TGB proteins and finally, a coat protein (see FIG. 6A). MaMV can be additionally characterised as producing 3 major viral RNAs during infection: one large genomic RNA, and two subgenomic species migrating as RNA of approximately 2000 and 800 nucleotides respectively (see FIG. 6B).

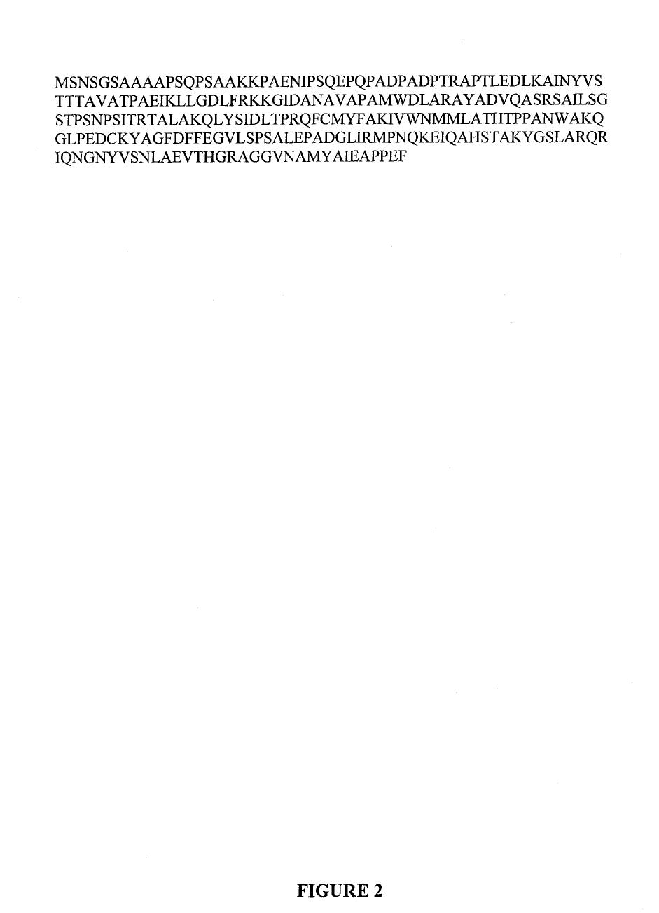

MaMV can be further characterised as having a coat protein having an amino acid sequence substantially identical to the sequence as set forth in SEQ ID NO:2 (see also FIG. 2).

As is known in the art, viral species are often represented by a number of different strains that share the same characteristics. The present invention, therefore, encompasses strain variants of the MaMV described in Example 1 that have the same physical characteristics and a genome sequence that shares at least about 80% sequence identity with SEQ ID NO:1. In one embodiment of the invention, strain variants have the same physical characteristics as the MaMV described in Example 1 and a genome sequence that shares at least about 90% sequence identity with SEQ ID NO:1. In one embodiment of the invention, strain variants have the same physical characteristics as the MaMV described in Example 1 and a genome sequence that shares at least about 95% sequence identity with SEQ ID NO:1.

### MaMV Virus-Like Particles (VLPs)

The MaMV VLPs of the present invention are preferably derived from MaMV coat protein. By “derived from” it is meant that the VLP comprises coat proteins that have an amino acid sequence substantially identical to the sequence of the wild-type MaMV coat protein and may optionally include one or more antigens attached to the coat protein, as described in more detail below. The MaMV coat protein comprised by the VLP, therefore, can be the wild-type coat protein or a recombinant version thereof, and may have the sequence of the wild-type protein or a modified version of this sequence that is capable of multimerization and self-assembly to form a VLP. VLPs comprising combinations of coat proteins having wild-type and modified sequences are also contemplated.

MaMV VLPs are formed from MaMV coat proteins that retain the ability to multimerise and self-assemble. When assembled, each VLP comprises a long helical array of coat protein subunits. The number of coat proteins comprised by the VLP can vary, for example, between about 40 and about 1600.

The VLPs of the present invention can be prepared from a plurality of coat proteins having identical amino acid sequences, such that the final VLP when assembled comprises identical coat protein subunits, or the VLP can be prepared from a plurality of coat proteins having different amino acid sequences, such that the final VLP when assembled comprises variations in its coat protein subunits.

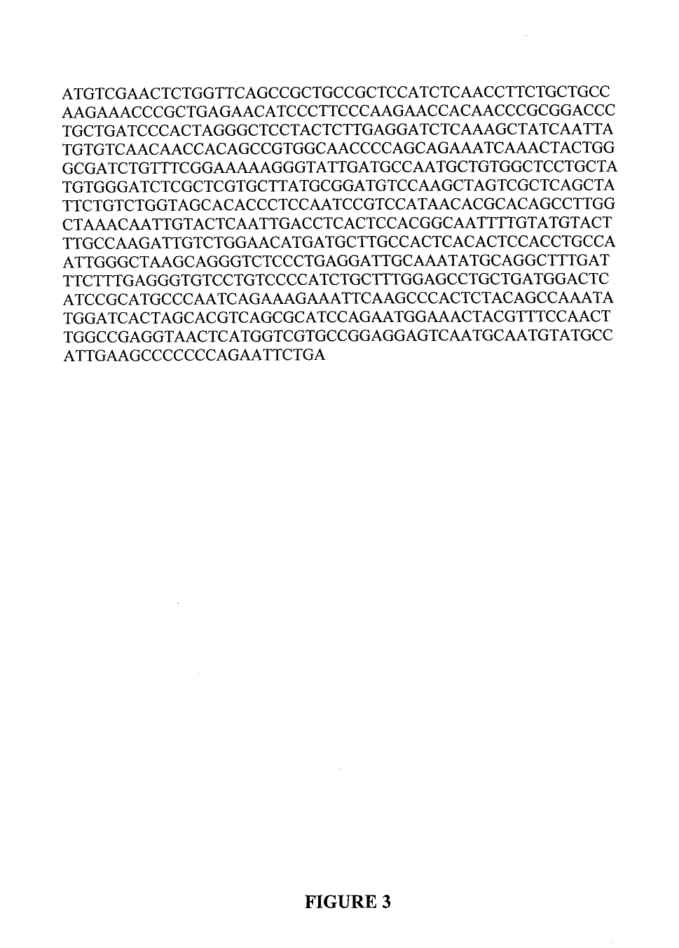

The coat protein used to form the VLP can be a full length coat protein, or a portion of the full-length coat protein, which is capable of multimerising to form a VLP. The coat protein sequence can be the naturally-occurring (wild-type) sequence, or it can be a genetically modified version of the wild-type sequence, for example, comprising one or more amino acid deletions, insertions, replacements and the like, provided that the modified coat protein retains the ability to multimerise and assemble into a VLP as described herein. The amino acid sequence of the coat protein of the MaMV described in Example 1 is provided herein as SEQ ID NO:2 (see FIG. 2) and is also accessible from GenBank (see GenBank Accession No. ABG48664). The nucleotide sequence of the MaMV coat protein is also is provided herein as SEQ ID NO:3 (see FIG. 3) and is accessible from GenBank (see GenBank Accession No. DQ660333 (nucleotides 6057-6788)).

As noted above, the amino acid sequence of the coat protein comprised by the VLP need not correspond exactly to the wild-type sequence as set forth in SEQ ID NO:2, i.e. it may be a modified sequence. For example, the sequence of the coat protein may be genetically modified by substitution, insertion or deletion of one or more amino acid residues so that the residue at the altered site(s) does not correspond to the wild-type (reference) sequence. For example, the nucleotide sequence encoding the coat protein may be modified by addition of an enzyme cleavage site resulting in a modification of the encoded coat protein, for example, by addition of between one and several amino acids at or near the C-terminus, the N-terminus, or both. One skilled in the art will appreciate, however, that such mutations will not be extensive and should not prevent the modified coat protein from multimerising and assembling into a VLP. Such a mutation may, however, improve the ability of the MaMV coat protein to self assemble and/or lead to formation of longer VLPs as previously shown for PapMV coat protein (Laliberté et al., 2008, FEBS Journal, 275:1474-1484). The ability of a modified version of the MaMV coat protein to assemble into VLPs can be assessed, for example, by electron microscopy following standard techniques, such as the exemplary methods set out in the Examples provided herein.

In general, suitable modified coat proteins comprise an amino acid sequence that is at least about 80% identical to the wild-type sequence as set forth in SEQ ID NO:2. In one embodiment, a suitable modified coat protein comprises an amino acid sequence that is at least about 85% identical to the wild-type sequence as set forth in SEQ ID NO:2. In another embodiment, suitable modified coat proteins comprise an amino acid sequence that is at least about 90% identical to the wild-type sequence as set forth in SEQ ID NO:2. In other embodiments, suitable modified coat proteins comprise an amino acid sequence that is at least about 91%, at least about 92%, at least about 93%, at least about 94%, at least about 95%, at least about 96%, at least about 97%, at least about 98% or at least about 99% identical to the wild-type sequence as set forth in SEQ ID NO:2.

Coat proteins that are fragments of the wild-type protein that retain the ability to multimerise and assemble into a VLP (i.e. are “functional” fragments) are also contemplated by the present invention. For example, a functional fragment may comprise a deletion of one or more amino acids from the N-terminus, the C-terminus, or the interior of the protein, or a combination thereof. In general, functional fragments comprise at least about 150 contiguous amino acids of the sequence as set forth in SEQ ID NO:2. In one embodiment of the present invention, functional fragments comprise at least about 180 contiguous amino acids of the sequence as set forth in SEQ ID NO:2. In another embodiment, functional fragments comprise at least about 190 contiguous amino acids of the sequence as set forth in SEQ ID NO:2. In other embodiments, functional fragments comprise at least about 200 contiguous amino acids, at least 210 contiguous amino acids, at least 220 contiguous amino acids, at least 225 contiguous amino acids, and at least 230 contiguous amino acids of the sequence as set forth in SEQ ID NO:2. Deletions made at the N-terminus or C-terminus of the protein should generally delete 25 amino acids or less in order to retain the ability of the protein to multimerise. For example, in one embodiment, deletions made at the N-terminus delete 20 amino acids or less. In other embodiments, deletions made at the N-terminus delete 15 amino acids or less, 12 amino acids or less, 10 amino acids or less, 8 amino acids or less, and 5 amino acids or less.

In one embodiment of the invention, the VLP comprises a recombinant version of the MaMV coat protein. In another embodiment, the VLP comprises a genetically modified version of the MaMV coat protein.

When the coat protein comprises a modified sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include, for example, alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another example of a grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

The nucleic acid sequence encoding the coat protein likewise need not correspond precisely to the wild-type sequence but may vary by virtue of the degeneracy of the genetic code and/or such that it encodes a modified amino acid sequence as described above. In one embodiment of the present invention, therefore, the nucleic acid sequence encoding the coat protein is at least about 70% identical to the sequence as set forth in SEQ ID NO:3. In another embodiment, the nucleic acid sequence encoding the coat protein is at least about 75% identical to the sequence as set forth in SEQ ID NO:3. In another embodiment, the nucleic acid sequence encoding the coat protein is at least about 80% identical to the sequence as set forth in SEQ ID NO:3. In other embodiments, the nucleic acid sequence encoding the coat protein is at least about 85% or at least about 90% identical to the sequence as set forth in SEQ ID NO:3.

As described in more detail below, the VLP coat protein may optionally be genetically fused to one or more antigens, an affinity peptide or other short peptide sequence, or a combination thereof, to facilitate attachment of one or more antigens.

### Antigens

The immunogenic compositions may optionally comprise one or more antigens, which can be separate from the MaMV or MaMV VLP, or conjugated to the MaMV or MaMV VLP Immunogenic compositions comprising both conjugated and non-conjugated antigens are also contemplated. In this latter context, the non-conjugated antigens can be considered as additional isolated antigens (AIAs). The AIAs may be the same as or different than the conjugated antigen(s). Conjugation can be, for example, by genetic fusion with the coat protein, or binding via covalent, non-covalent or affinity means. In one embodiment of the invention, the one or more antigens can be provided in the form of a commercially available vaccine.

A wide variety of antigens suitable for the development of vaccines are known in the art. Appropriate antigens for inclusion in the immunogenic compositions of the invention can be readily selected by one skilled in the art based on, for example, the desired end use of the immunogenic composition such as the disease or disorder against which it is to be directed, the format of composition, whether the composition is intended for use as a multivalent or monovalent vaccine and/or the animal to which it is to be administered.

For example, the antigen can be derived from an agent capable of causing a disease or disorder in an animal, such as a cancer, infectious disease, allergic reaction, or autoimmune disease, or it can be an antigen suitable for use to induce an immune response against drugs, hormones or a toxin-associated disease or disorder. The antigen may be derived from a pathogen known in the art, such as, for example, a bacterium, virus, protozoan, fungus, parasite, or infectious particle, such as a prion, or it may be a tumour-associated antigen, a self-antigen or an allergen. Alternatively, the antigen may comprise a B-cell epitope or a T-cell epitope of an antigen.

The antigen(s) can be included in the immunogenic composition in various formats, as noted above and described in more detail below. The antigen(s) for incorporation into the immunogenic compositions can thus vary in size depending on the format selected. The antigen may be, for example, a peptide, a protein, a nucleic acid, a polysaccharide, a small molecule, or a combination thereof up to and including a whole pathogen or a portion thereof, for example, a live, inactivated or attenuated version of a pathogen.

When the immunogenic composition is to comprise more than one antigen, the antigens selected for inclusion in the composition can be the same, or they can be different, and may be derived from a single source or from a plurality of sources. The antigens can each have a single epitope capable of triggering a specific immune response, or each antigen may comprise more than one epitope.

The antigen may comprise epitopes recognised by surface structures on T cells, B cells, NK cells, macrophages, Class I or Class II APC associated cell surface structures, or a combination thereof. In one embodiment, the present invention contemplates that the immunogenic compositions are especially useful for small and/or weakly immunogenic antigens.

Antigens for inclusion in the immunogenic compositions of the invention may also be selected from pathogens or other sources of interest by art known methods and screened for their ability to induce an immune response in an animal using standard immunological techniques known in the art. For example, methods for prediction of epitopes within an antigenic protein are described in Nussinov R and Wolfson H J, Comb Chem High Throughput Screen (1999) 2(5):261, and methods of predicting CTL epitopes are described in Rothbard et al., EMBO J. (1988) 7:93-100 and in de Groot M S et al., Vaccine (2001) 19(31):4385-95. Other methods are described in Rammensee H-G. et al., Immunogenetics (1995) 41:178-228 and Schirle M et al., Eur J Immunol (2000) 30(18):2216-2225.

Useful viral antigens for example, include those derived from members of the families Adenoviradae; Arenaviridae (for example, Ippy virus and Lassa virus); Birnaviridae; Bunyaviridae; Caliciviridae; Coronaviridae; Filoviridae; Flaviviridae (for example, yellow fever virus, dengue fever virus and hepatitis C virus); Hepadnaviradae (for example, hepatitis B virus); Herpesviradae (for example, human herpes simplex virus 1); Orthomyxoviridae (for example, influenza virus A, B and C); Paramyxoviridae (for example, mumps virus, human metapneumovirus, measles virus and respiratory syncytial virus); Picornaviridae (for example, poliovirus and hepatitis A virus); Poxyiridae; Reoviridae; Retroviradae (for example, BLV-HTLV retrovirus, HIV-1, HIV-2, bovine immunodeficiency virus and feline immunodeficiency virus); Rhabodoviridae (for example, rabies virus), and Togaviridae (for example, rubella virus). In one embodiment, the immunogenic compositions comprise one or more antigens derived from a major viral pathogen such as the various hepatitis viruses, human immunodeficiency virus (HIV), various influenza viruses, West Nile virus, respiratory syncytial virus, rabies virus, human papilloma virus (HPV), Epstein Barr virus (EBV), polyoma virus, or SARS coronavirus.

Viral antigens derived from the hepatitis viruses, including hepatitis A virus (HAV), hepatitis B virus (HBV), hepatitis C virus (HCV), the delta hepatitis virus (HDV), hepatitis E virus (HEY) and hepatitis G virus (HGV), are known in the art. For example, antigens can be derived from HCV core protein, E1 protein, E2 protein, NS3 protein, NS4 protein or NS5 protein, from HBV HbsAg antigen or HBV core antigen, and from HDV delta-antigen (see, for example, U.S. Pat. No. 5,378,814). U.S. Pat. Nos. 6,596,476; 6,592,871; 6,183,949; 6,235,284; 6,780,967; 5,981,286; 5,910,404; 6,613,530; 6,709,828; 6,667,387; 6,007,982; 6,165,730; 6,649,735 and 6,576,417, for example, describe various antigens based on HCV core protein.

Non-limiting examples of known antigens from the herpesvirus family include those derived from herpes simplex virus (HSV) types 1 and 2, such as HSV-1 and HSV-2 glycoproteins gB, gD and gH.

Non-limiting examples of HIV antigens include antigens derived from gp120, antigens derived from various envelope proteins such as gp160 and gp41, gag antigens such as p24gag and p55gag, as well as proteins derived from the pol, env, tat, vif rev, nef vpr, vpu and LTR regions of HIV. The sequences of gp120 from a multitude of HIV-1 and HIV-2 isolates, including members of the various genetic subtypes of HIV are known (see, for example, Myers et al., Los Alamos Database, Los Alamos National Laboratory, Los Alamos, N. Mex. (1992); and Modrow et al., J. Virol. (1987) 61:570 578).

Non-limiting examples of other viral antigens include those from varicella zoster virus (VZV), Epstein-Barr virus (EBV) and cytomegalovirus (CMV) including CMV gB and gH; and antigens from other human herpesviruses such as HHV6 and HHV7 (see, for example Chee et al. (1990) Cytomegaloviruses (J. K. McDougall, ed., Springer-Verlag, pp. 125 169; McGeoch et al. (1988) J. Gen. Virol. 69:1531 1574; U.S. Pat. No. 5,171,568; Baer et al. (1984) Nature 310:207 211; and Davison et al. (1986) J. Gen. Virol. 67:1759 1816.)

Antigens can also be derived from the influenza virus, for example, from the haemagglutinin (HA), neuramidase (NA), nucleoprotein (NP), the matrix proteins (M1 and M2), the polymerase acidic protein (PA) and the polymerase basic proteins subunits (PB1, PB2). The sequences of these proteins are known in the art and are readily accessible from GenBank database maintained by the National Center for Biotechnology Information (NCBI). Suitable antigenic fragments of HA, NP and the matrix proteins include, but are not limited to, the haemagglutinin epitopes: HA 91-108, HA 307-319 and HA 306-324 (Rothbard, Cell, 1988, 52:515-523), HA 458-467 (J. Immunol. 1997, 159(10): 4753-61), HA 213-227, HA 241-255, HA 529-543 and HA 533-547 (Gao, W. et al., J. Virol., 2006, 80:1959-1964); the nucleoprotein epitopes: NP 206-229 (Brett, 1991, J. Immunol. 147:984-991), NP335-350 and NP380-393 (Dyer and Middleton, 1993, In: Histocompatibility testing, a practical approach (Ed.: Rickwood, D. and Hames, B. D.) IRL Press, Oxford, p. 292; Gulukota and DeLisi, 1996, Genetic Analysis: Biomolecular Engineering, 13:81), NP 305-313 (DiBrino, 1993, PNAS 90:1508-12); NP 384-394 (Kvist, 1991, Nature. 348:446-448); NP 89-101 (Cerundolo, 1991, Proc. R. Soc. Lon. 244:169-7); NP 91-99 (Silver et al, 1993, Nature. 360: 367-369); NP 380-388 (Suhrbier, 1993, J. Immunology 79:171-173); NP 44-52 and NP 265-273 (DiBrino, 1993, ibid.); and NP 365-380 (Townsend, 1986, Cell. 44:959-968); the matrix protein (M1) epitopes: M1 2-22, M1 2-12, M1 3-11, M1 3-12, M1 41-51, M1 50-59, M1 51-59, M1 134-142, M1 145-155, M1 164-172, M1 164-173 (all described by Nijman, 1993, Eur. J. Immunol. 23:1215-1219); M1 17-31, M1 55-73, M1 57-68 (Carreno, 1992, Mol Immunol 29:1131-1140); M1 27-35, M1 232-240 (DiBrino, 1993, ibid.), M1 59-68 and M1 60-68 (Eur. J. Immunol. 1994, 24(3): 777-80); and M1 128-135 (Eur. J. Immunol. 1996, 26(2): 335-39).\; PA 412-422 (Heiny A T, et al., 2007, PLoS ONE, 21; 2(11):e1190); PB1 15-51 (Heiny A T, et al., 2007, PLoS ONE, 21; 2(11):e1190) and PB2 685-696 (Heiny A T, et al., 2007, PLoS ONE, 21; 2(11):e1190).

Other examples include the F3 peptide derived from the HA protein, which has the sequence: KAYSNCYPYDVPDY (SEQ ID NO:72) (Lu et al., 2002, Int. Arch. Allergy Immunol, 127: 245-250), and CTL epitopes from the M1 protein having the sequence: SPLTKGILGFVFTLTVPSE (SEQ ID NO:73), and GILGFVFTL (SEQ ID NO:60).

Other related antigenic regions and epitopes are also known. For example, fragments of the influenza ion channel protein (M2), including the M2e peptide (the extracellular domain of M2). The sequence of this peptide is highly conserved across different strains of influenza. An example of a M2e peptide sequence is shown in Table 1 as SEQ ID NO:4. Variants of this sequence have been identified and some are also shown in Table 1.

TABLE 1
M2e Peptide and Variations Thereof

| Region | | | SEQ |
|---|---|---|---|
| of | | | ID |
| M2 | Sequence | Viral Strain | NO |
| 2-24 | SLLTEVETPIRN | Human H1N1 e.g. | 4 |
| | EWGCRCNDSSD | A/USRR/90/77 and | |
| | | A/WSN/33 | |
| 2-24 | SLLTEVETPIRN | N/A* | 5 |
| | EWGCRCNGSSD | | |
| 2-24 | SLLTEVETPTKN | N/A* | 6 |
| | EWDCRCNDSSD | | |
| 2-24 | SLLTEVETPTRN | Equine H3N8 | 7 |
| | GWECKCSDSSD | A/equine/Massachussetts/ | |
| | | 213/2003 | |
| 2-24 | SLLTEVETPTRN | H5N1 A/Vietnam/1196/04 | 8 |
| | EWECRCSDSSD | | |
| 1-24 | MSLLTEVETPIR | Human H1N1 e.g. | 74 |
| | NEWGCRCNDSSD | A/USRR/90/77 and | |
| | | A/WSN/33 | |
| 1-24 | HSLLTEVETPTR | Avian H5N1 | 75 |
| | NEWECRCSDSSD | A/Vietnam/1196/04 | |
| 1-24 | MSLLTEVETPTR | H3N8, Horse-Dog | 76 |
| | NGWECKCSDSSD | A/equine/Massachussetts/ | |
| | | 213/2003 | |
| 1-24 | MSLLTEVETPTR | H9N2, | 77 |
| | NGWGCRCSDSSD | A/chicken/Osaka/aq69/2001 | |
| 1-24 | MSLLTEVETPTR | Mutant H1N1 I/T | 78 |
| | NEWGCRCSDSSD | | |

*see U.S. Pat. application No. 2006/0246092

The entire M2e sequence or a partial M2e sequence may be used, for example, a partial sequence that is conserved across the variants, such as fragments within the region defined by amino acids 2 to 10, or the conserved epitope EVETPIRN [SEQ ID NO:9] (amino acids 6-13 of the M2e sequence). The 6-13 epitope has been found to be invariable in 84% of human influenza A strains available in GenBank. Variants of this sequence that were also identified include EVETLTRN [SEQ ID NO:10] (9.6%), EVETPIRS [SEQ ID NO:11] (2.3%), EVETPTRN [SEQ ID NO:12] (1.1%), EVETPTKN [SEQ ID NO:13] (1.1%) and EVDTLTRN [SEQ ID NO:14], EVETPIRK [SEQ ID NO:15] and EVETLTKN [SEQ ID NO:16] (0.6% each) (see Zou, P., et al., 2005, Int Immunopharmacology, 5:631-635; Liu et al. 2005, Microbes and Infection, 7:171-177).

Other useful antigens include live, attenuated and inactivated viruses such as inactivated polio virus (Jiang et al., J. Biol. Stand., (1986) 14:103-9), attenuated strains of Hepatitis A virus (Bradley et al., J. Med. Virol., (1984) 14:373-86), attenuated measles virus (James et al., N. Engl. J. Med., (1995) 332:1262-6), and epitopes of pertussis virus (for example, ACEL-IMUNE™ acellular DTP, Wyeth-Lederle Vaccines and Pediatrics).

Antigens can also be derived from unconventional viruses or virus-like agents such as the causative agents of kuru, Creutzfeldt-Jakob disease (CJD), scrapie, transmissible mink encephalopathy, and chronic wasting diseases, or from proteinaceous infectious particles such as prions that are associated with mad cow disease, as are known in the art.

Useful bacterial antigens include for example superficial bacterial antigenic components, such as lipopolysaccharides, capsular antigens (proteinacious or polysaccharide in nature), or flagellar components and may be obtained or derived from known causative agents responsible for diseases such as Diptheria, Pertussis, Tetanus, Tuberculosis, Bacterial or Fungal Pneumonia, Cholera, Typhoid, Plague, Shigellosis or Salmonellosis, Legionaire's Disease, Lyme Disease, Leprosy, Malaria, Hookworm, Onchocerciasis, Schistosomiasis, Trypamasomialsis, Lesmaniasis, Giardia, Amoebiasis, Filariasis, Borrelia, and Trichinosis.

Examples of antigens derived from gram-negative bacteria of the family Enterobacteriaceae include, but are not limited to, the S. typhi Vi (capsular polysaccharide) antigen, the E. coli K and CFA (capsular component) antigens and the E. coli fimbrial adhesin antigens (K88 and K99). Examples of antigenic proteins include the outer membrane proteins (Omps), also known as porins (Secundino et al., 2006, Immunology 117:59); related porins such as the S. typhi iron-regulated outer membrane protein (IROMP, Sood et al., 2005, Mol Cell Biochem 273:69-78), and heat shock proteins (HSPs) including, but not limited to S. typhi HSP40 (Sagi et al., 2006, Vaccine 24:7135-7141). Non-limiting examples of antigenic porins include OmpC and OmpF, which are found in numerous Salmonella and Escherichia species. Orthologues of OmpC and OmpF are also found in other Enterobacteriaceae and are suitable antigenic proteins for the purposes of the present invention. In addition, Omp1B (Shigella flexneri), OmpC2 (Yersinia pestis), OmpD (S. enterica), OmpK36 (Klebsiella pneumonie), OmpN (E. coli) and OmpS (S. enterica) may be suitable, based on conserved regions of sequences found in the porin proteins of the Enterobacteriaceae family (Diaz-Quinonez et al., 2004, Infect. and Immunity 72:3059-3062).

The sequences of antigenic proteins from various enterobacteria are known in the art and are readily accessible from GenBank database maintained by the National Center for Biotechnology Information (NCBI). For example, GenBank Accession No. P0A264 and GenBank Accession No. NP_{—}804453: OmpC (S. enterica subsp. enterica serovar Typhi Ty2); GenBank Accession No. CAD05399: OmpF precursor protein (S. enterica subsp. enterica serovar Typhi CT18); GenBank Accession No. 16761195: OmpC (S. enterica serovar Typhimurium); GenBank Accession No. 47797: OmpC (S. enterica serovar Typhi); GenBank Accession No. 8953564: OmpC (S. enterica serovar Minnesota); GenBank Accession No. 19743624: OmpC (S. enterica serovar Dublin); GenBank Accession No. 19743622: OmpC (S. enterica serovar Gallinarum); GenBank Accession No. 26248604: OmpC (E. coli); GenBank Accession No. 24113600: Omp1B (Shigella flexneri); GenBank Accession No. 16764875: OmpC2 (Yersinia pestis); GenBank Accession No. 16764916: OmpD (S. enterica Serovar Typhimurium); GenBank Accession No. 151149831: OmpK36 (Klebsiella pneumonie); GenBank Accession No. 3273514: OmpN (E. coli), and GenBank Accession No. 16760442: OmpS (S. enterica serovar Typhi).

Various tumour-associated antigens are known in the art. Representative examples include, but are not limited to, Her2 (breast cancer); GD2 (neuroblastoma); EGF-R (malignant glioblastoma); CEA (medullary thyroid cancer); CD52 (leukemia); human melanoma protein gp100 (for example, antigens comprising the epitope: IMDQVPFSV (SEQ ID NO:59)); human melanoma protein melan-A/MART-1; human Dickkopf1 (DKK1) protein, human angiomotin (Amot), NA17; NA17-A nt protein; p53 protein; various MAGEs (melanoma associated antigen E), including MAGE 1, MAGE 2, MAGE 3 (HLA-A1 peptide) and MAGE 4; various tyrosinases (HLA-A2 peptide); mutant ras; p97 melanoma antigen; Ras peptide and p53 peptide associated with advanced cancers; the HPV 16/18 and E6/E7 antigens associated with cervical cancers; MUC1-KLH antigen associated with breast carcinoma; CEA (carcinoembryonic antigen) associated with colorectal cancer, and the PSA antigen associated with prostate cancer.

Useful allergens include, but are not limited to, allergens from pollens, animal dander, grasses, moulds, dusts, antibiotics, stinging insect venoms, as well as a variety of environmental, drug and food allergens. Common tree allergens include pollens from cottonwood, popular, ash, birch, maple, oak, elm, hickory, and pecan trees. Common plant allergens include those from rye, ragweed, English plantain, sorrel-dock and pigweed, and plant contact allergens include those from poison oak, poison ivy and nettles. Common grass allergens include Timothy, Johnson, Bermuda, fescue and bluegrass allergens. Common allergens can also be obtained from moulds or fungi such as Alternaria, Fusarium, Hormodendrum, Aspergillus, Micropolyspora, Mucor and thermophilic actinomycetes. Penicillin, sulfonamides and tetracycline are common antibiotic allergens. Epidermal allergens can be obtained from house or organic dusts (typically fungal in origin), from insects such as house mites (dermatphagoides pterosinyssis), or from animal sources such as feathers, and cat and dog dander. Common food allergens include milk and cheese (diary), egg, wheat, nut (for example, peanut), seafood (for example, shellfish), pea, bean and gluten allergens. Common drug allergens include local anesthetic and salicylate allergens, and common insect allergens include bee, hornet, wasp and ant venom, and cockroach calyx allergens.

Particularly well characterized allergens include, but are not limited to, the dust mite allergens Der pI and Der pII (see, Chua, et al., J. Exp. Med., 167:175 182, 1988; and, Chua, et al., Int. Arch. Allergy Appl. Immunol., (1990) 91:124-129), T cell epitope peptides of the Der pII allergen (see, Joost van Neerven, et al., J. Immunol., (1993) 151:2326-2335), the highly abundant Antigen E (Amb aI) ragweed pollen allergen (see, Rafnar, et al., J. Biol. Chem., (1991) 266:1229-1236), phospholipase A2 (bee venom) allergen and T cell epitopes therein (see, Dhillon, et al., J. Allergy Clin. Immunol., (1992) 42), white birch pollen (Betvl) (see, Breiteneder, et al., EMBO, (1989) 8:1935-1938), the Fel dI major domestic cat allergen (see, Rogers, et al., Mol. Immunol., (1993) 30:559-568), tree pollen (see, Elsayed et al., Scand. J. Clin. Lab. Invest. Suppl., (1991) 204:17-31) and the multi-epitopic recombinant grass allergen rKBG8.3 (Cao et al. Immunology (1997) 90:46-51). These and other suitable allergens are commercially available and/or can be readily prepared following known techniques.

Antigens relating to conditions associated with self antigens are also known to those of ordinary skill in the art. Representative examples of such antigens include, but are not limited to, lymphotoxins, lymphotoxin receptors, receptor activator of nuclear factor kB ligand (RANKL), vascular endothelial growth factor (VEGF), vascular endothelial growth factor receptor (VEGF-R), interleukin-5, interleukin-17, interleukin-13, CCL21, CXCL12, SDF-1, MCP-1, endoglin, resistin, GHRH, LHRH, TRH, MIF, eotaxin, bradykinin, BLC, tumour Necrosis Factor alpha and amyloid beta peptide, as well as fragments of each which can be used to elicit immunological responses.

Useful toxins are generally the natural products of toxic plants, animals, and microorganisms, or fragments of these compounds. Such compounds include, for example, aflatoxin, ciguautera toxin, pertussis toxin and tetrodotoxin.

Antigens useful in relation to recreational drug addiction are known in the art and include, for example, opioids and morphine derivatives such as codeine, fentanyl, heroin, morphine and opium; stimulants such as amphetamine, cocaine, MDMA (methylenedioxymethamphetamine), methamphetamine, methylphenidate, and nicotine; hallucinogens such as LSD, mescaline and psilocybin; cannabinoids such as hashish and marijuana, other addictive drugs or compounds, and derivatives, by-products, variants and complexes of such compounds.

In one embodiment of the present invention, the antigen(s) included in the immunogenic composition are protein antigens. A protein antigen can be a full-length protein, a substantially full-length protein (for example, a protein comprising a N-terminal and/or C-terminal deletion of about 25 amino acids or less), an antigenic fragment of the protein, or a combination thereof. The full-length protein can be, when applicable, a precursor form of the protein or the mature (processed) form of the protein. The protein may be post-translationally modified, for example, a glycoprotein or lipoprotein. An antigenic fragment can comprise one, or a plurality of epitopes, and thus may range in size from a peptide of a few amino acids (for example, at least 4 amino acids) to a polypeptide several hundred amino acids in length. In one embodiment of the invention, antigenic fragments suitable for inclusion in the immunogenic compositions are between about 4 amino acids and about 250 amino acids in length. In another embodiment, antigenic fragments suitable for inclusion in the immunogenic compositions are between about 5 amino acids and about 200 amino acids in length. In other embodiments, antigenic fragments suitable for inclusion in the immunogenic compositions are between about 5 amino acids and about 150 amino acids in length, between about 5 amino acids and about 100 amino acids in length, between about 5 amino acids and about 75 amino acids in length, between about 5 amino acids and about 70 amino acids in length, between about 5 amino acids and about 60 amino acids in length, and between about 5 amino acids and about 50 amino acids in length.

As noted above, in one embodiment of the invention, the one or more antigens included in the immunogenic compositions can be in the form of a commercial vaccine. Various human vaccines are known in the art and include, but are not limited to, vaccines against:

Bacillus anthracis (anthrax), such as BioThrax® (BioPort Corporation);
Haemophilus influenzae type b (Hib), such as, ActHIB® (Sanofi-aventis), PedvaxHIB® (Merck) and HibTITER® (Wyeth);
hepatitis A, such as, Havrix® (GlaxoSmithKline) and Vaqta® (Merck);
hepatitis B, such as, Engerix-B® (GlaxoSmithKline) and Recombivax HB® (Merck);
Herpes zoster (shingles), such as, Zostavax® (Merck);
human papillomavirus (HPV), such as, Gardasil® (Merck);
influenza, such as, Fluarix® and Fluviral® (GlaxoSmithKline), FluLaval® (ID Biomedical Corp of Quebec); FluMist® (intranasal) (Medimmune), Fluvirin® (Chiron), and Fluzone® (Sanofi-aventis);
Japanese encephalitis, such as, JE-Vax® (Sanofi-aventis);
measles, such as, Attenuvax® (Merck);
Meningococcal meninigitis, such as, Menomune® Meningococcal Polysaccharide (Sanofi-aventis);
mumps, such as, Mumpsvax® (Merck);
pneumococcal disease, such as, Pneumovax 23® Pneumococcal Polysaccharide (Sanofi-aventis) and Prevnar® Pneumococcal Conjugate (Wyeth);
polio, such as, Ipol® (Sanofi-aventis) and Poliovax® (Sanofi-Pasteur);
rabies, such as, BioRab® (BioPort Corporation), RabAvert® (Chiron) and Imovax® Rabies (Sanofi-aventis);
rotavirus, such as, RotaTeq® (Merck);
rubella, such as, Meruvax II® (Merck);
S. typhi (typhoid fever), such as, Typhim Vi® (Sanofi-aventis) and Vivotif® Berna (oral) (Berna);
tuberculosis (BCG), such as, TheraCys® and ImmuCyst® (Sanofi-aventis); TICE® BCG and Oncotice™ (Organon Teknika Corporation); Pacis™; and Mycobax® (Sanofi-Pasteur);
vaccinia (smallpox), such as, Dryvax® (Wyeth);
varicella (chickenpox), such as, Varivax® (Merck);
yellow fever, such as, YF-Vax® (Sanofi-aventis);
hepatitis A/hepatitis B, such as, Twinrix® (GlaxoSmithKline);
hepatitis B and Hib, such as, Comvax® (Merck);
tetanus/Hib, such as, ActHIB® (Sanofi-Pasteur);
diphtheria/Hib, such as, HibTITER® (Wyeth Pharmaceuticals);
Hib/meningitis, such as, PedVaxHIB (Merck & Co);
meningitis/diptheria, such as, Menactra® Meningococcal Conjugate (Sanofi-Pasteur);
tetanus/dipheria (Td), such as, Decavac® (Sanofi-aventis);
diphtheria/tetanus/pertussis (DTaP/DT or DTaP), such as, Daptacel® and Tripedia® (Sanofi-aventis) and Infanrix® (GlaxoSmithKline);
tetanus/diphtheria/pertussis (Tdap), such as, Boostrix® (GlaxoSmithKline) and Adacel® (Sanofi-Pasteur);
DTaP/Hib, such as, TriHIBit® (Sanofi-aventis);
DTaP/polio/hepatitis B, such as Pediarix® (GlaxoSmithKline);
measles/mumps/rubella (MMR), such as, M-M-R II (Merck) and
measles/mumps/rubella/chickenpox, such as, ProQuad® (Merck).

Examples of vaccines for veterinarian use include, but are not limited to, vaccines against Lawsonia intracellularis (for example, Enterisol and Ileitis), Porphyromonas gulae, and P. denticanis (for example, Periovac), Streptococcus equi (for example, Equilis StrepE), Chlamydophila abortus (for example, Ovilis and Enzovax), Mycoplasma synoviae (for example, Vaxsafe MS), Mycoplasma gallisepticum (for example, Vaxsafe MG), Bordetella avium (for example, Art Vax), Actinobacillus pleuropneumoniae (for example, PleuroStar APP), Actinobacillus pleuropneumoniae (for example, Porcilis APP), Salmonella (for example, Megan Vac1 and MeganEgg), Brucella abortus (for example, RB-51), Eimeria spp. (for example, Coccivac, Immucox, Paracox, Advent, and Nobilis Cox ATM), Eimeria spp. (for example, Inovocox), E. tenella (for example, Livacox), Toxoplasma gondii (for example, Ovilis and Toxovax), Pseudorabies virus (for example, Suvaxyn Aujeszky), Classical swine fever virus (for example, Porcilis Pesti and Bayovac CSF E2), Equine influenza virus (for example, PROTEQ-FLU and Recombitek), Newcastle disease virus (for example, Vectormune FP-ND), Avian influenza virus (for example, Poulvac FluFend I AI H5N3 RG), Avian influenza virus (for example, Trovac AI H5), Rabies virus (for example, Raboral and Purevax Feline Rabies), Feline leukemia virus (for example, EURIFEL FeLV), Canine parvovirus 1 (for example, RECOMBITEK Canine Parvo), Canine coronavirus (for example, RECOMBITEK Corona MLV), Canine distemper virus (for example, RECOMBITEK rDistemper and PUREVAXFerret Distemper), IHN virus (for example, Apex-IHN). Other examples of veterinarian vaccines include reproduction control vaccines such as LHRH (for example, Vaxstrate, Improvac, Equito, Canine gonadotropinreleasing factor immunotherapeutic, and GonaCon) and Androstenedione (for example, Fecundin, Androvax and Ovastim).

In one embodiment of the invention, the antigens included in the immunogenic composition are in the form of a known influenza vaccine. Most commercially available influenza vaccines are split virus vaccines in which the influenza virus has been treated with an organic solvent to remove surface glycoproteins, subunit vaccines, or live attenuated virus vaccines, or a combination thereof. In general, commercial influenza vaccines are trivalent in that they provide protection against three strains of influenza, for example, for the 2007-2008 season the strains were A/Solomon Islands/3/2006 (H1N1)-like, A/Wisconsin/67/2005 (H3N2)-like, and B/Malaysia/2506/2004-like.

Influenza vaccines that are presently commercially available include, but are not limited to, Fluzone® and Vaxigrip® (Sanofi-aventis), Fluvirin® (Novartis Vaccine), Fluarix®, FluLaval® and Fluviral S/F® (GlaxoSmithKline), Afluria (CSL Biotherapies), FluMist® (MedImmune), and Influvac™ (Solvay Pharma).

### Antigen-MaMV and Antigen MaMV VLP Combinations

As noted above, the one or more antigens comprised by the immunogenic composition can be conjugated to a coat protein of the MaMV or MaMV VLP, or they may be present in the composition in a non-conjugated form (i.e. simply combined with the MaMV or MaMV VLP), or they may be present in both conjugated and non-conjugated form. Conjugation can be, for example, by genetic fusion with the coat protein, or binding via covalent, non-covalent or affinity means. Combination of the antigen(s) with the MaMV or VLP, however, should not interfere with the recognition of the antigen by the host's immune system or the ability of the MaMV or VLP to potentiate an immune response.

In accordance one embodiment of the present invention, the one or more antigens comprised by the iummunological composition are conjugated to a coat protein of a MaMV VLP. As the VLP comprises multiple copies of self-assembled coat protein, attaching the antigen to the coat protein allows presentation of multiple antigens on the surface of the VLP.

In order to allow presentation of the antigen on the surface of the VLP and enhance immune recognition of the antigen, the antigen is preferably attached to a region of the coat protein that is disposed on the outer surface of the VLP. Thus the antigen can be inserted near, or attached at, the amino- (N-) or carboxy- (C-) terminus of the coat protein, or it can be inserted into, or attached to, an internal region of the coat protein which is disposed on the outer surface of the VLP. The ability of the antigen-conjugated coat protein to assemble with other fusion coat proteins or with wild-type coat protein to form a VLP should be retained and this ability can be readily tested by art known methods, including those described herein. In one embodiment, the antigen is attached at, or proximal to, the C-terminus of the coat protein.

In accordance with one embodiment of the invention, the immunogenic composition comprises MaMV coat protein genetically fused to one or more antigens. In order to avoid the possibility of the antigen interfering with the ability of the MaMV coat protein-antigen fusion to self-assemble into a VLP, antigens selected for genetic fusion to the MaMV coat protein are typically about 50 amino acids or less in length, for example, about 45 amino acids or less in length.

If desired a spacer can be included between the antigen and the coat protein. Suitable spacers for this purpose are known in the art and include, for example, short amino acid sequences of between about 3 and about 10 amino acids. In general, amino acid spacers in this context are composed of neutral amino acids, such glycine, leucine, valine and isoleucine. In one embodiment, when the antigen is genetically fused to the MaMV coat protein, the fusion comprises a peptide spacer of between about 3 and about 10 neutral amino acids.

Larger antigens can be readily incorporated into the immunogenic composition by simple combination with the MaMV or VLP, or by chemical cross-linking or affinity attachment, as described in more detail below.

The antigen(s) can be chemically cross-linked to the coat protein, for example, by covalent or non-covalent (such as, ionic, hydrophobic, hydrogen bonding, or the like) attachment. The antigen and/or coat protein can be modified to facilitate such cross-linking as is known in the art, for example, by addition of a functional group or chemical moiety to the protein and/or antigen, for example at the C- or N-terminus or at an internal position. Exemplary modifications include the addition of functional groups such as S-acetylmercaptosuccinic anhydride (SAMSA) or S-acetyl thioacetate (SATA), or addition of one or more cysteine residues. Other cross-linking reagents are known in the art and many are commercially available (see, for example, catalogues from Pierce Chemical Co. and Sigma-Aldrich). Examples include, but are not limited to, diamines, such as 1,6-diaminohexane, 1,3-diamino propane and 1,3-diamino ethane; dialdehydes, such as glutaraldehyde; succinimide esters, such as ethylene glycol-bis(succinic acid N-hydroxysuccinimide ester), disuccinimidyl glutarate, disuccinimidyl suberate, N-(g-Maleimidobutyryloxy)sulfosuccinimide ester and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; 3-maleimidopropionic acid N-hydroxysuccinimide ester, and the like. Many of the above-noted cross-linking agents incorporate a spacer that distances the affinity moiety from the VLP. The use of other spacers is also contemplated by the invention. Various spacers are known in the art and include, but are not limited to, 6-aminohexanoic acid; 1,3-diamino propane; 1,3-diamino ethane; and short amino acid sequences, such as polyglycine sequences, of 1 to 5 amino acids.

To facilitate covalent attachment of the one or more antigen to the coat protein of the VLP, the coat protein can be genetically fused to a short peptide or amino acid linker that is exposed in the surface of the VLP and provides an appropriate site for chemical attachment of the antigen. For example, short peptides comprising cysteine residues, or other amino acid residues having side chains that are capable of forming covalent bonds (for example, acidic and basic residues) or that can be readily modified to form covalent bonds as known in the art. The amino acid linker or peptide can be, for example, between one and about 20 amino acids in length. In one embodiment, the coat protein is fused with a short peptide comprising one or more lysine residues, which can be covalently coupled, for example with a cysteine residue in the antigen through the use of a suitable cross-linking agent as described above.

In a further embodiment of the present invention, the antigen is attached via an affinity moiety present on the coat protein. In accordance with this embodiment, the MaMV VLP comprises an affinity moiety, such as a peptide, that is exposed on the surface of the VLP following self-assembly, and which is capable of specifically binding to the antigen. The affinity moiety may be genetically fused (in the case of a peptide or protein fragment), or covalently or non-covalently attached to the MaMV or VLP. Binding of the antigen to the affinity moiety should not interfere with the recognition of the antigen by the host's immune system. The affinity moiety can be capable of binding a whole protein or it may be capable of binding a protein fragment or peptide.

Examples of suitable affinity moieties include, but are not limited to, antibodies and antibody fragments (such as Fab fragments, Fab′ fragments, Fab′-SH, fragments F(ab′)₂ fragments, Fv fragments, diabodies, and single-chain Fv (scFv) molecules), streptavidin (to bind biotin labelled antigens), affinity peptides or protein fragments that specifically bind the antigen.

Suitable peptides or antibodies (including antibody fragments) for use as affinity moieties can be selected by art-known techniques, such as phage or yeast display techniques. The peptides can be naturally occurring, recombinant, synthetic, or a combination of these. For example, the peptide can be a fragment of a naturally occurring protein or polypeptide. The term peptide also encompasses peptide analogues, peptide derivatives and peptidomimetic compounds. Such compounds are well known in the art and may have advantages over naturally occurring peptides, including, for example, greater chemical stability, increased resistance to proteolytic degradation and/or reduced antigenicity.

Suitable peptides for use as affinity moities can range from about 3 amino acids in length to about 50 amino acids in length. In accordance with one embodiment of the invention, the affinity binding peptide is at least 5 amino acids in length. In accordance with another embodiment of the invention, the affinity binding peptide is at least 7 amino acids in length. In accordance with another embodiment of the invention, the affinity binding peptide is between about 5 and about 50 amino acids in length. In accordance with another embodiment of the invention, the affinity binding peptide is between about 7 and about 50 amino acids in length. In other embodiments of the invention, the affinity binding peptide is between about 5 and about 45 amino acids in length, between about 5 and about 40 amino acids in length, between about 5 and about 35 amino acids in length and between about 5 and about 30 amino acids in length. In accordance with a specific embodiment of the invention, the affinity binding peptide is 3, 4, 5, 6, 7, 8, 9, 10, 11, 12, 13, 14 or 15 amino acids in length. As would be understood by a worker skilled in the art, the length of the peptide selected for binding the antigen to the affinity moiety should not interfere with the ability of the MaMV VLP to self-assemble or with recognition of the antigen, once bound, by the host's immune system.

Affinity moieties comprised by the MaMV or VLP can be single peptides or can be a tandem or multiple arrangement of peptides. A spacer can be included between the affinity moiety and the coat protein if desired in order to facilitate the binding of large antigens. Suitable spacers include short stretches of neutral amino acids, such as glycine. For example, a stretch of between about 3 and about 10 neutral amino acids.

Phage display can be used to select specific peptides that bind to an antigenic protein of interest using standard techniques (see, for example, Current Protocols in Immunology, ed. Coligan et al., J. Wiley & Sons, New York, N.Y.) and/or commercially available phage display kits (for example, the Ph.D. series of kits available from New England Biolabs, and the T7-Select® kit available from Novagen). An example of selection of peptides by phage display is also provided in Example 8, below.

### Preparation of MaMV and MaMV VLPs

The present invention provides for MaMV VLPs derived from a recombinant MaMV coat protein, and for immunogenic compositions comprising MaMV or MaMV VLPs. The invention further provides MaMV VLPs that comprise one or more antigens, or an affinity moiety, in genetic fusion with the coat proteins. These recombinant coat proteins are capable of multimerisation and assembly into VLPs. Methods for genetically fusing the antigens, or affinity peptides for linking to antigens, to the coat protein are well known in the art and representative examples are described below and in the Examples section. Methods of chemical cross-linking various molecules to proteins are also well known in the art and can be employed.

### Malva Mosaic Virus

As described in the Examples section provided herein, MaMV can be isolated from Malva neglecta Wallr. (common mallow). The virus can be readily propagated on M. neglecta or on plant species from the family Chenopodiaceae, for example, Chenopodium quinoa as described in Example 1.

The virus can be readily propagated by, in brief, rubbing healthy leaves of Chenopodium quinoa (4 leaves stage) with about 10-100 μg of purified virus in an appropriate volume of carrier, for example, about 40 μL. Alternatively, juice obtained from grinding infected leaves in water in a mortar (for example, 1 g infected leaves in 2 ml of water) can be used to inoculate the healthy plant. Carborendum (an abrasive) is gently deposited at the surface of the healthy leaf prior rubbing with the virus or juice. The infection is initiated by rubbing gently with the finger to create microlesions through which the virus will enter the plant cells. Approximately ten minutes after rubbing, the inoculated leaf is rinsed with water to remove the carborendum and any residue. The inoculated plant is grown for approximately an additional 5-14 days until the infection shows symptoms of mosaic or local lesions (depending of the environmental conditions of light and photoperiod). Usually, plants are grown with 16 hours light and 8 hours darkness at 22° C. during the light periods and at 16° C. during the dark periods. Plants can be fertilised using 20-20-20 fertiliser according to the manufacturer's protocol. In general, approximately 100-1000 g of infected plant leaves are required for virus purification.

Virions can be isolated from infected leaves by standard potexvirus isolation techniques (see, for example, AbouHaidar M G, et al. (1998) Methods Mol Cell Biol 81:131-143, and Tremblay, M.-H., et al., (2006). FEBS J 273:14-25). A further example of a suitable method is as follows. Infected leaves are harvested and homogenized in a suitable buffer, followed by filtration and/or centrifugation to remove debris. The resulting suspension is treated with butanol and optionally a detergent, then stirred on ice. The solution is next centrifuged and the resulting pellet resuspended in an appropriate buffer. The pellet is homogenized then re-centrifuged. Virions are pelleted from the supernatant by ultracentrifugation on a sucrose cushion and the resulting pellet resuspended in an appropriate buffer using the homogenizer. The virus solution can be cleaned by an additional centrifugation step if desired prior to being passed through a 0.45 μM syringe filter.

Purified virions can be stored under refrigeration, for example at 4° C.

### MaMV VLPs

Recombinant MaMV coat proteins suitable for preparation of VLPs in accordance with the invention can be readily prepared using standard genetic engineering techniques by the skilled worker provided with the sequence of the wild-type coat protein. Methods of genetically engineering proteins are well known in the art (see, for example, Ausubel et al. (1994 & updates) Current Protocols in Molecular Biology, John Wiley & Sons, New York). The amino acid sequence of the wild-type MaMV coat protein (see SEQ ID NO:2) and the nucleotide sequence encoding the wild-type protein (see SEQ ID NO:3) are provided herein and are also publicly available from GenBank as noted above.

Isolation and cloning of the nucleic acid sequence encoding the wild-type protein can be achieved using standard techniques (see, for example, Ausubel et al., ibid.), for example, by extracting RNA from MaMV by standard techniques and then synthesizing cDNA from the RNA template (for example, by RT-PCR). MaMV can be purified from infected plant leaves that show mosaic symptoms by standard techniques as noted above (see, also Example 1 provided herein).

Alternatively, the gene encoding the coat protein can be constructed artificially using standard techniques. For example, several (for example, 18 to 20) overlapping phosphorylated oligonucleotides of about 80 nucleotides in length and representing the entire gene sequence can be synthesized using standard techniques. Each nucleotide should overlap at their 5′ and 3′ ends, for example by about 20 nucleotides, with the exception of the final 5′ oligo that overlaps only at the 3′ end and the final 3′ oligo that overlaps only at the 5′ end. The oligonucleotides can be pooled in an appropriate buffer (for example 10 mM Tris/HCl pH 8 and 25 mM NaCl), heated at 90° C. for 15 min and cooled to room temperature slowly to allow annealing between the oligonucleotides and generation of the full-length MaMV CP gene. The addition of T4 DNA ligase for 1 hour in ligase buffer will complete the assembly of the oligonucleotides. Optionally, the oligonucleotides comprising the 5′ and the 3′ end of the MaMV CP gene can contain unique restriction sites which can be used to clone the annealed DNA an appropriate vector, such as a bacterial plasmid. The vector can be used to transform an appropriate host cell, such as plasmid E. coli to amplify the plasmid and complete the ligation of the oligonucleotides. Annealing of all the oligonucleotides may be improved by annealing them 2 by 2 sequentially, followed by annealing of each pair of oligonucleotides 2 by 2, and so on, until the full-length gene is generated.

Alternatively, the full length MaMV CP gene can be amplified by polymerase chain reaction (PCR) before cloning into an appropriate plasmid (pET-3D as an example) for expression of the protein in E. coli (BL21 (DE3) for example).

The full-length gene sequence may also be obtained through the services of one of a number of commercial companies that construct synthetic genes (for example, GenScript Corp. (Piscataway, N.Y.), Geneart AG (Regensberg, Bavaria) and Molecular Cloning Laboratories (San Francisco, Calif.)).

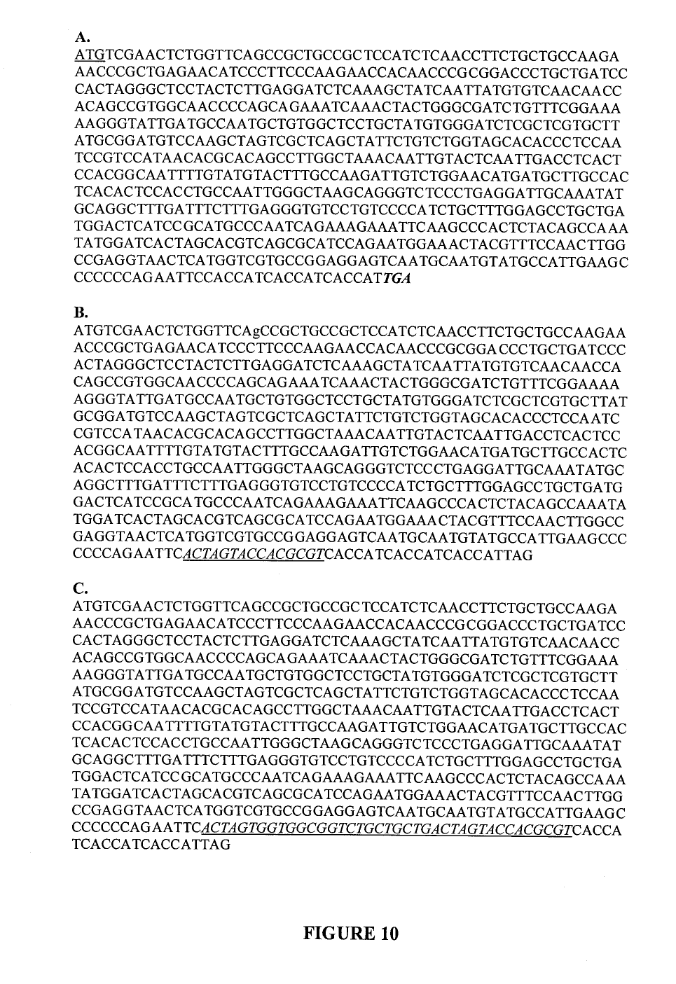

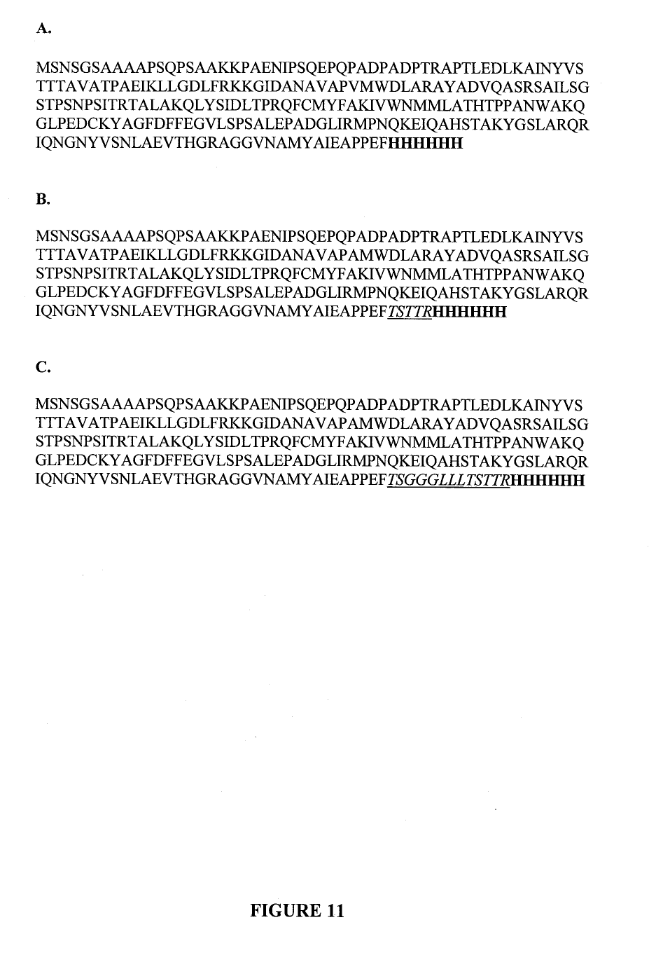

The nucleic acid sequence encoding the coat protein is then inserted directly, or after one or more subcloning steps, into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described in more detail below. An example of a vector comprising the coat protein of MaMV (the plasmid pMaMV-CP-6H (pET-3D comprising the MaMV coat protein)) is described in Example 6. The nucleotide sequence of the MaMV coat protein gene contained in plasmid pMaMV-CP-6H is provided in FIG. 10A (SEQ ID NO:23) and the amino acid sequence of the encoded coat protein is provided in FIG. 11A (SEQ ID NO:24).

Optionally, the nucleic acid sequence encoding the coat protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

As noted above, the coat proteins can also be engineered to produce fusion proteins comprising one or more antigens, affinity peptides and/or spacer peptides fused to the coat protein. Methods for making fusion proteins are well known to those skilled in the art. DNA sequences encoding a fusion protein can be inserted into a suitable expression vector as noted above.

One of ordinary skill in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

One skilled in the art will also understand that the expression vector may further include regulatory elements, such as transcriptional elements, required for efficient transcription of the DNA sequence encoding the coat or fusion protein. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a coat protein or fusion protein. One skilled in the art will appreciate that selection of suitable regulatory elements is dependent on the host cell chosen for expression of the protein and that such regulatory elements may be derived from a variety of sources, including bacterial, fungal, viral, mammalian or insect genes.

If desired, the expression vector may additionally contain heterologous nucleic acid sequences that facilitate the purification of the expressed protein as is known in the art. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The resulting heterologous amino acid sequence can be removed from the expressed protein prior to use according to methods known in the art. Alternatively, the heterologous amino acid sequence can be retained on the protein provided that it does not interfere with subsequent assembly of the protein into VLPs. In one embodiment of the present invention, the coat protein is expressed as a histidine tagged protein. The heterologous amino acid sequence can be located at the carboxyl terminus or the amino terminus of the coat protein.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (for example, E. coli, A. salmonicida or B. subtilis) or in a eukaryotic host (for example, Saccharomyces or Pichia; mammalian cells, for example, COS, NIH 3T3, CHO, BHK, 293, or HeLa cells; or insect cells). In one embodiment of the invention, the recombinant coat protein is expressed in plant or bacterial cells. In another embodiment, the recombinant coat protein is expressed in bacterial cells. In another embodiment, the recombinant coat protein is expressed in E. coli cells.

If desired, the expressed protein can be purified from the host cells by standard techniques known in the art (see, for example, in Current Protocols in Protein Science, ed. Coligan, J. E., et al., Wiley & Sons, New York, N.Y.) and sequenced by standard peptide sequencing techniques using either the intact protein or proteolytic fragments thereof to confirm the identity of the protein.

In accordance with one embodiment of the invention, the recombinant coat proteins are capable of multimerisation and assembly into VLPs. In general, VLP assembly takes place in the host cell expressing the coat protein. The VLPs can be isolated from the host cells by standard techniques, such as those described in the Examples section provided herein. The VLPs can optionally be further purified by standard techniques, such as chromatography, to remove contaminating host cell proteins or other compounds, such as LPS.

In one embodiment of the present invention, the coat proteins assemble to provide a virus or pseudovirus in the host cell and can be used to produce infective virus particles which comprise nucleic acid and protein. This can enable the infection of adjacent cells by the infective virus or pseudovirus particle and expression of the protein therein. In this embodiment, the host cell used to replicate the virus or pseudovirus can be a plant cell, insect cell, mammalian cell or bacterial cell that will allow the virus to replicate. The cell may be a natural host cell for the virus from which the virus-like particle is derived, but this is not necessary. The host cell can be infected initially with virus or pseudovirus in particle form (i.e. in assembled rods comprising nucleic acid and a protein) or alternatively in nucleic acid form (i.e. RNA such as viral RNA; cDNA or run-off transcripts prepared from cDNA) provided that the virus nucleic acid used for initial infection can replicate and cause production of whole virus particles.

### Production of Stock MaMV or MaMV VLP

Stocks of recombinant MaMV or VLP can be prepared by standard techniques. For example, MaMV or a pseudovirus comprising the recombinant coat protein can be propagated in an appropriate host, such as Chenopodium quinoa, such that sufficient MaMV or pseudovirus can be harvested.

Stocks of MaMV VLPs can be prepared from an appropriate host cell, such as E. coli, transformed or transfected with an expression vector prepared as described encoding the recombinant coat protein that makes up the VLP. The host cells are then cultured under conditions that favour the expression of the encoded protein, as is known in the art. The expressed coat protein can multimerise and assemble into VLPs in the host cell and can be isolated from the cells by standard techniques as described above, for example, by rupturing the cells and submitting the cell lysate to one or more chromatographic purification steps.

Stocks of the MaMV and MaMV VLPs can be stored in a refrigerator, for example at 4° C.

### Characteristics of Recombinant and Modified Coat Proteins

Recombinant coat proteins and coat proteins to which antigens, affinity peptides and/or spacer peptides have been attached can be analysed for their ability to multimerize and self-assemble into a VLP by standard techniques. For example, by visualising the purified protein by electron microscopy (see, for example, Example 6). VLP formation may also be determined by ultracentrifugation, and circular dichroism (CD) spectrophotometry may be used to compare the secondary structure of the recombinant or modified proteins with the WT virus (see, for example, Tremblay, et al., FEBS J, 2006, 273:14-25).

Stability of the VLPs, and of MaMV, can be determined if desired by techniques known in the art, for example, by SDS-PAGE and trypsin degradation analyses (see, for example, Tremblay, et al., 2006, supra).

### Evaluation of Efficacy

The ability of the immunogenic compositions of the present invention to induce an immune response in an animal can be tested by art-known methods, such as those described below and in the Examples. For example, the MaMV, MaMV VLP or immunogenic composition comprising same can be administered to a suitable animal model, for example by subcutaneous injection or intranasally, and the development of antibodies evaluated.

Cellular immune response can also be assessed by techniques known in the art. For example, the cellular immune response can be determined by evaluating processing and cross-presentation of an epitope expressed on a MaMV VLP to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines (see, for example, Leclerc, D., et al., J. Viral, 2007, 81(3):1319-26).

In order to determine the efficacy of immunogenic compositions comprising MaMV or MaMV VLPs and one or more antigens as a vaccine, challenge studies can be conducted. Such studies involve the inoculation of groups of a test animal (such as mice, rats or ferrets) with an immunogenic composition of the invention by standard techniques. Control groups comprising non-inoculated animals and/or animals inoculated with a commercially available vaccine, or other positive control, are set up in parallel. After an appropriate period of time post-vaccination, the animals are challenged with the naturally-occurring antigen-containing substance or organism. Blood samples collected from the animals pre- and post-inoculation, as well as post-challenge are then analyzed for an antibody response to the virus. Suitable tests for the antibody response include, but are not limited to, Western blot analysis and Enzyme-Linked Immunosorbent Assay (ELISA). The animals can also be monitored for development of symptoms of the condition associated with the antigen-containing substance or organism.

Similarly, immunogenic compositions comprising tumour-associated antigens can be tested for their prophylactic effect by inoculation of test animals and subsequent challenge by transplanting cancer cells into the animal, for example subcutaneously, and monitoring tumour development in the animal. Alternatively, the therapeutic effect of the immunogenic composition can be tested by administering the composition to the test animal after implantation of cancer cells and establishment of a tumour and monitoring the growth and/or metastasis of the tumour.

### Pharmaceutical Compositions

As noted above, the immunogenic compositions comprising MaMV or a MaMV VLP and optionally one or more antigens may further optionally comprise a suitable carrier, excipient or the like, and/or other standard components of pharmaceutical compositions that improve the stability, palatability, pharmacokinetics, bioavailability or the like, of the composition. In one embodiment of the invention, the immunogenic composition is formulated for use as an adjuvant. In another embodiment, the immunogenic composition is formulated for use as a vaccine.

The compositions can be formulated for administration by a variety of routes. For example, the compositions can be formulated for oral, topical, rectal, nasal or parenteral administration or for administration by inhalation or spray. The term parenteral as used herein includes subcutaneous injections, intravenous, intramuscular, intrathecal, intrasternal injection or infusion techniques. Intranasal administration to the subject includes administering the pharmaceutical composition to the mucous membranes of the nasal passage or nasal cavity of the subject. In one embodiment of the present invention, the compositions are formulated for topical, rectal or parenteral administration or for administration by inhalation or spray, for example by an intranasal route. In another embodiment, the compositions are formulated for parenteral administration.

The compositions preferably comprise an effective amount of the immunogenic composition of the invention. The term “effective amount” as used herein refers to an amount of the composition required to produce a detectable immune response. The effective amount of immunogenic composition for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the invention, the unit dose comprises between about 5 μg and about 10 mg of coat protein. In another embodiment, the unit dose comprises between about 10 μm and about 10 mg of coat protein. In another embodiment, the unit dose comprises between about 5 μg and about 5 mg of coat protein. In other embodiments, the unit dose comprises between about 10 μg and about 5 mg of coat protein, between about 10 μg and about 2 mg of coat protein, between about 15 μg and about 5 mg of coat protein and between about 20 μg and about 5 mg of coat protein. One or more doses may be used to immunise the animal, and these may be administered on the same day or over the course of several days or weeks.

As noted above, the immunogenic compositions of the present invention may comprise a plurality of antigens (in a conjugated and/or non-conjugated form), and may thus provide a multivalent vaccine formulation. Multivalent vaccine compositions that comprise a plurality of VLPs, each conjugated to a different antigen are also contemplated. Multivalent vaccine formulations include bivalent and trivalent formulations in addition to vaccines having higher valencies.

In certain embodiments, vaccine formulations comprising a plurality of (i.e. two or more) different antigens may also provide improved protection due to the higher number of epitopes in the formulation. The antigens can be included in the formulation in conjugated form (for example, by way of a plurality of different VLPs each conjugated to a different antigen), or in non-conjugated form, or in both conjugated and non-conjugated forms.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the immunogenic composition in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the immunogenic composition is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Pharmaceutical compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the immunogenic composition in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the immunogenic composition in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the immunogenic composition in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the immunogenic composition with one or more sweetening agents, for example glycerol, propylene glycol, sorbitol or sucrose. Such formulations can also optionally contain one or more demulcents, preservatives, flavouring agents and/or colouring agents.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the composition of the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum albumin or skimmed milk) together with a suitable buffer (e.g. phosphate buffer). The pH and exact concentration of the various components of the composition may be adjusted according to well-known parameters.

Further, one or more compounds having adjuvant activity may be optionally added to the vaccine composition. Suitable adjuvants include, for example, aluminium hydroxide, phosphate or oxide; oil-emulsions (e.g. of Bayol F® or Marcol52®); saponins, or vitamin-E solubilisate. Opsonised vaccine compositions are also encompassed by the present invention, for example, vaccine compositions comprising antibodies isolated from animals or humans previously immunised with the vaccine. Recombinant antibodies based on antibodies isolated from animals or humans previously immunised with the vaccine could also be used to opsonise the vaccine composition.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

In addition, one or more conventional adjuvants may optionally be added to the composition. Suitable adjuvants include, for example, alum adjuvants (such as aluminium hydroxide, phosphate or oxide); oil-emulsions (e.g. of Bayol F® or Marcol52®); saponins, or vitamin-E solubilisate. Virosomes are also known to have adjuvant properties (Adjuvant and Antigen Delivery Properties of Virosomes, Glück, R., et al., 2005, Current Drug Delivery, 2:395-400), as have S. typhi porin proteins (for example, OmpC), and can optionally be included in the compositions of the invention.

In addition, the invention contemplates that, when the immunogenic composition comprises MaMV VLPs fused to an antigen, that the composition may also include MaMV VLPs derived from an unfused coat protein in order to increase the overall adjuvant effect of the VLPs in the composition.

The compositions may optionally comprise an opsonin, for example, antibodies isolated from animals or humans previously immunised with the antigen, MaMV or MaMV VLPs. Recombinant antibodies based on antibodies isolated from animals or humans previously immunised with the antigen, MaMV or MaMV VLPs could also be used as opsonins.

Also encompassed by the present invention are compositions comprising MaMV or MaMV VLPs in combination with a commercially available vaccine, as described above.

### Uses of the Immunogenic Compositions

The present invention provides for a number of uses of MaMV, MaMV VLPs and immunogenic compositions comprising same. Non-limiting examples include the use of the immunogenic composition as an adjuvant, immunostimulant or as a vaccine. MaMV VLPs conjugated to one more antigens can also be used to screen for antibodies to the antigen(s). The present invention thus provides methods for inducing an immune response in an animal by administering the immunogenic composition, as well, the use of the immunogenic compositions for the preparation of medicaments, such as adjuvants, immunostimulants, vaccines and/or pharmaceutical compositions.

The immunogenic compositions of the invention are suitable for use in humans as well as non-human animals, including domestic and farm animals. The administration regime for the immunogenic composition need not differ from any other generally accepted vaccination programs. For example, a single administration of the immunogenic composition in an amount sufficient to elicit an effective immune response may be used or, alternatively, other regimes of initial administration of the immunogenic composition followed by boosting with antigen alone or with the immunogenic composition may be used. Similarly, boosting with either the immunogenic composition or antigen may occur at times that take place well after the initial administration if antibody titres fall below acceptable levels. The exact mode of administration of the immunogenic composition will depend for example on the components of the composition (for example, whether the composition comprises an antigen or is being provided as an adjuvant), the animal to be treated and the desired end effect of the treatment. Appropriate modes of administration can be readily determined by the skilled practitioner.

When the immunogenic composition comprises non-conjugated antigen(s), the MaMV or VLP component can be administered concomitantly with the antigen(s), or it can be administered prior or subsequent to the administration of the antigen, depending on the needs of the subject in which an immune response is desired.

The immunogenic composition can be used prophylactically, for example to prevent infection by a virus, bacteria or other infectious particle, or development of a disease or tumour, or it may be used therapeutically to ameliorate the effects of a disease or disorder, for example, associated with an infection or a cancer. In one embodiment of the invention, the immunogenic composition is used prophylactically. In this context, the immunogenic composition can comprise one or more antigens, or the immunogenic composition may comprise MaMV or a MaMV VLP alone, which can be sufficient to induce resistance to infections, for example low level infections, or disease.

The immunogenic composition can be used in the prevention or treatment of a variety of diseases or disorders depending on the antigen selected for inclusion in, or use with, the composition. Non-limiting examples include influenza (using antigens from various influenza viruses), typhoid fever (using antigens from S. typhi), HCV infections (using HCV antigens), HBV infections (using HBV antugens), HAV infections (using HAV antigens), HIV infections (using HIV antigens), polio (using poliovirus antigens), diptheria (using antigens derived from diptheria toxin), EBV infections (using EBV antigens), allergic reactions (using various allergens) and cancer (using various tumour-associated antigens). Other uses include, for example, prevention or treatment of inflammatory diseases (for example, arthritis) and infections by avian flu virus, human respiratory syncytial virus, Dengue virus, measles virus, herpes simplex virus, human papillomavirus, pseudorabies virus, swine rotavirus, swine parvovirus, Newcastle disease virus, foot and mouth disease virus, hog cholera virus, African swine fever virus, infectious bovine rhinotracheitis virus, infectious laryngotracheitis virus, La Crosse virus, neonatal calf diarrhea virus, bovine respiratory syncytial virus, bovine viral diarrhea virus, Mycoplasma hyopneumoniae, Streptococcal bacteria, Gonococcal bacteria, Enterobacteria and parasites (for example, leishmania or malaria).

The immunogenic compositions of the invention are also suitable for use as multivalent vaccines, for example, when the compositions comprise a plurality of antigens from different disease-causing agents.

The immunogenic compositions can also be used in conjunction with a conventional vaccine to improve the efficacy of the vaccine, or to provide a multivalent vaccine. Non-limiting examples of commercially available vaccines that could be used in this context are provided above. The commercially available vaccine may be a human vaccine or a vaccine intended for veterinary use.

In one embodiment, the invention also provides for the use of the immunogenic compositions for vaccination of subjects who have previously been vaccinated with a potexvirus-based vaccine. This approach can ensure that the efficacy of the second vaccine is not diminished due to interaction with antibodies raised to the potexvirus in the first vaccine. For example, a PapMV-based vaccine platform has been described (see, International Patent Application No. PCT/CA03/00985 (WO 2004/004761) and U.S. patent application Ser. No. 11/556,678 (US2007/0166322)). Immunisation of animals or humans with a vaccine adjuvanted with PapMV VLPs may generate large amount of antibodies directed to the PapMV coat protein that is the main component of the adjuvant. Immunisation of the same patient with the same PapMV platform could result in resident antibodies potentially limiting the immune response to the newly administered vaccine by binding directly to the PapMV platform. As such, the use of a second vaccine based on MaMV or MaMV VLPs will be useful in avoiding this effect and helps to ensure a more efficient vaccination program.

The present invention also provides for the use of the MaMV VLPs conjugated to one or more antigens as a screening agent, for example, to screen for antibodies to the antigen(s). The VLPs can be readily adapted to conventional immunological techniques such as an enzyme-linked immunosorbant assay (ELISA) or Western blotting and are thus useful in diagnostic and research contexts.

### Kits

The present invention additionally provides for kits comprising MaMV, MaMV VLPs or an immunogenic composition of the invention for use as an adjuvant or vaccine. Where the MaMV, MaMV VLPs or immunogenic composition are intended for use with a separate antigen preparation, including a commercial vaccine, the kit can optionally include the antigen preparation.

Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the adjuvant or vaccine.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

Screening kits containing MaMV VLPs conjugated to one or more antigens for use in antibody detection are also provided. The kits can be diagnostic kits or kits intended for research purposes. Individual components of the kit would be packaged in separate containers and, associated with such containers, can be a notice in the form prescribed by a governmental agency regulating the manufacture, use or sale of biological products, which notice reflects approval by the agency of manufacture, use or sale of the biological product. The kit may optionally contain instructions or directions outlining the method of use for the immunogenic composition.

To gain a better understanding of the invention described herein, the following examples are set forth. It will be understood that these examples are intended to describe illustrative embodiments of the invention and are not intended to limit the scope of the invention in any way.

### EXAMPLES

### Example 1

### Malva Mosaic Virus Amplification, Purification and RNA Extraction

A new potexvirus, Malva Mosaic virus (MaMV) was isolated from Malva neglecta Wallr. (common mallow). The initial infected Malva neglecta plants was collected in the area surrounding Summerland, British Columbia, Canada. Mosaic symptoms and vein clearing induced by the viral infection are the only symptoms observed on M. neglecta (see FIG. 4A).

Infection of Chenopodium quinoa with the virus produced a strong mosaic pattern that moved systemically throughout the whole plant (see FIG. 4B). C. quinoa was, therefore, used as the propagation host and was used for the collection of a large amount of infected leaves for purification of the virus. When the inoculum was taken from C. quinoa infected leaves for propagation in C. quinoa, the development of local lesions was noticed (see FIG. 4C). It is likely that the induction of local lesions is related to the concentration of the virus in the sap (more virus in infected C. quinoa leaves) used for inoculation which would explain the differences of the symptoms on the same host.

MaMV was amplified and purified as follows. Infected leaves of Malva neglecta were harvested from the area of Summerland, B.C. and sent to the inventor's laboratory in Québec. Leaf powder was obtained by crushing the infected leaves of Malva neglecta in a pestle/mortar in liquid nitrogen. The powder was resuspended in water and inoculated on the propagation host Chenopodium quinoa by rubbing the leaves with carborundum. Leaves showed symptoms of infection (i.e. mosaic and vein clearing on the infected leaves) 2-3 weeks following inoculation. Infected leaves were homogenized in 100 mM sodium phosphate buffer pH 7.6 containing 10 mM EDTA and 0.1% sodium bisulfate. The homogenate was filtered through layers of cheesecloth and centrifuged at 7,800 g for 20 min. 0.5% Triton X-100 and 2% butanol were added drop by drop to the supernatant and stirred on ice for 60 min. The solution was centrifuged 20 min at 7,800 g. The supernatant was then ultracentrifuged at 100,000 g for 90 min and the resulting pellet resuspended in 100 mM sodium phosphate buffer pH 7.6. The pellet was homogenized with a Dyna-Mix homogenizer (Fisher Scientific at setting 2) before being recentrifuged at 7,800 g for 5 min. Virus was pelleted from the supernatant by an ultracentrifugation at 100,000 g for 3 hours on a 30% sucrose cushion and the pellet resuspended with the Dyna-Mix homogenizer in 10 mM Tris-HCl buffer pH 8.0. The virus solution was cleaned by a last centrifugation at 7,800 g for 5 min before being finally passed through a 0.45 μM syringe filter unit (Nalgene). The purified virus was kept at 4° C. until utilization for electron microscopy or viral RNA extraction.

Viral RNA was extracted from the virus solution by a phenol/chloroform extraction followed by an ethanol precipitation. Proteinase K treatment was also employed to remove any residual protein from the RNA. RNA was stored at −20° C. until required.

For electron microscopic analysis of the virus, purified virus particles were placed onto formvar/carbon-coated copper grid and stained with 1% methylamine tungstate. Samples were analyzed with JEOL transmission electron microscope. Electron microscopy analysis of the purified virus obtained from infected C. quinoa leaves revealed that the virus is a flexible filamentous virion of about 540×13 nm (FIG. 4, bar represents 50 nm), which is typical for a member of the potexviruses family (Adams et al., 2004. Arch. Virol. 149, 1045-1060).

### Example 2

### Malva Mosaic Virus Cloning and Sequencing

MaMV was cloned and sequenced for further study as described below.

First strand cDNA was synthesized from MaMV RNA prepared as described in Example 1 with random hexanucleotide or poly-dT primer using the Superscript™ first-strand synthesis system for RT-PCR (Invitrogen) according to the manufacturer's instructions. cDNA was then passed through a PCR purification kit column (QIAGEN) in order to remove unused primer before proceeding to the addition of a poly-deoxycytidine tail at the 3′ end of the cDNA using terminal transferase (New England Biolabs) according to the manufacturer's instructions. Poly-deoxyguanosine primer tagged with an Asc I or Pac I restriction site was hybridized to the cDNA for the synthesis of the second-strand using E. coli DNA polymerase Klenow fragment (New England Biolabs) according to the manufacturer's instructions. The resulting double-stranded DNA was purified by phenol/chloroform extraction and ethanol precipitation and then treated as described by the manufacturer with T4 DNA polymerase (New England Biolabs) in order to create blunt ended extremities. Blunt-ended DNA was digested with the appropriate restriction enzyme before being ligated in pNEB193 vector (New England Biolabs) overnight at 16° C. with T4 DNA ligase (New England Biolabs). The ligation reaction was transformed into E. coli DH5α and the transformed cells were grown on LB-agar plates with ampicillin (50 μg/ml). Plasmid DNA from resistant colonies was extracted using a QIAprep spin miniprep kit (QIAGEN) and then sequenced with an ABI 3730XL sequencer. Clones containing three overlapping fragments of the MaMV genomic sequence were obtained; one comprising the CP and the triple gene block, one comprising the rest of the replicase, and several clones comprising the 5′ end of MaMV (see FIG. 6A). The resulting DNA sequence corresponding to the MaMV genome is shown in FIG. 1 (SEQ ID NO:1).

### Example 3

### Identification of the 5′ End of Malva Mosaic Virus

The 5′-end sequence of MaMV was identified as follows. The genomic RNA (prepared as in Example 1) was first treated for 60 minutes at 37° C. with 2 U of Tobacco Acid Pyrophosphatase (Epicentre Biotechnologies) in 50 mM sodium acetate pH 6.0, 1 mM EDTA, 0.1% β-mercaptoethanol and 0.01% triton X-100 in order to remove the 5′-cap structure. The reactions were stopped by phenol-chloroform extraction (0.5 vol./0.5 vol.) and the RNA was ethanol precipitated before being reverse transcribed as described above, but using primer 0023-11, which hybridizes at position 498-520 of the genomic RNA (5′-ACATGTAAGCTAAACTAGTGTC-3′, SEQ ID NO:17). The oligonucleotide EMSA1 (5′-GTGATAAAGTTATGACCATAACCTATGTCGTAGGATATGCATTAACTAAT-3′, SEQ ID NO:18) was then added to the 3′-end of the cDNA using 20 U of RNA ligase under conditions described by the manufacturer (New England Biolabs). The second strand was then synthesized and amplified by PCR using the kit “Expand High Fidelity PCR System” as described by the manufacturer (Roche Diagnostics) in presence of the primer 0023-11 and EMSA2 (5′-ATTAGTTAATGCATATCCTACGACATAGGTTATGGTCATAACTTTATCAC-3′, SEQ ID NO:19), which is complementary to EMSA1. The DNA was denatured for 2 minutes at 94° C. before proceeding with amplification by PCR using 33 cycles consisting of: 45 seconds at 94° C., 45 seconds at 60° C. and 60 seconds at 72° C. Amplification products were directly cloned into the vector pCR2.1-TOPO using the TOPO TA cloning kit (Invitrogen). Ligation products were transformed into and amplified in E. coli DH5α, and plasmid DNA was extracted and sequenced as described in Example 2. Several clones were sequenced to precisely evaluate the 5′-end of the MaMV genome.

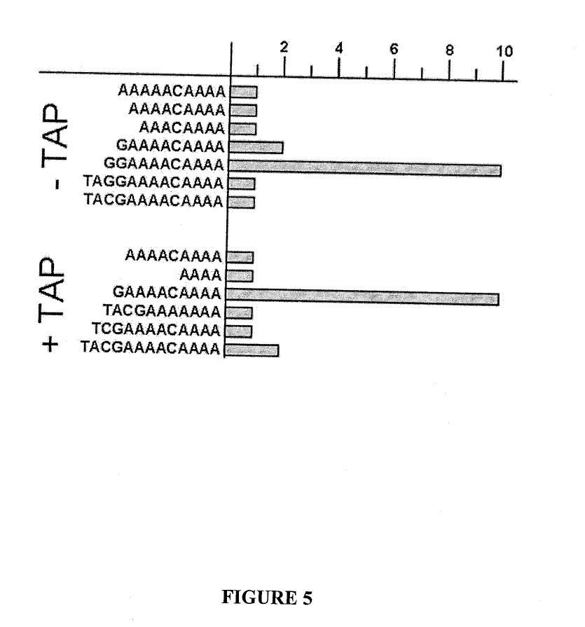

Analysis of the sequence of the 5′-end of the MaMV genome determined the following. The 5′ untranslated region (UTR) is a 81 nucleotide AC rich domain that is mostly unstructured, except for the presence of two putative stem loops of weak stability within nucleotides 26-36 and 48-78 respectively. Like most potexviruses, the predominant 5′-end motif observed with RNA treated with Tobacco Acid Pyrophosphatase (TAP) prior to reverse transcription is the consensus motif GAAAA (10 sequences out of 16) (FIG. 5). FIG. 5 shows the sequence obtained with RNA un-treated (-TAP) and pre-treated (+TAP) with the Tobacco Acid Pyrophosphatase prior to the reverse transcriptase reaction in the upper and lower panel respectively. The bars indicate the frequency that each sequence was found in all the clones sequenced. As shown in FIG. 5, when the Tobacco Acid Pyrophosphatase treatment was omitted, the predominant motif was GGAAAA as observed in ScaVX, CymMV and AlsVX (10 out of 17 sequences) (Wong et al., 1997. Arch. Virol. 142, 383-391; Kim et al., 1998. Mol. Cell. 8, 181-188; Chen et al., 2002. Arch Virol. 147, 683-693; Fuji et al., 2005. Arch Virol. 150, 2377-2385.). In the last case, the consensus GAAAA sequence was observed only twice. This discrepancy could be explained in part by the observation that most reverse transcriptases have terminal transferase and template switching activity. It has been shown that Superscript II, when tested with an in vitro transcribed RNA containing cap termini using the buffer conditions recommended by the manufacturer, will preferentially add one additional cytosine residue to the 3′ terminus of the cDNA (Schmidt et Mueller, 1999. Nucleic Acids Res. 27, e31.). Schmidt also observed that dCMP tailing by the reverse transcriptase is 10-fold more efficient with capped RNA templates compared to 5′-OH RNA. This observation could have explained the results observed here, since the cytosine residue added to the 3′-end of the cDNA would be read as an extra guanosine at the 5′-end of the RNA. This additional G residue observed with the non-treated RNA would be in fact artefactual due to the previously described terminal transferase activity of the reverse transcriptase and consequently, would not accurately represent the exact motif found at the 5′-end of the genomic RNA. Accordingly, it is likely that the results obtained with TAP-treated RNA reflect more accurately the sequence of the genome for the MaMV natural population found in hosts.

Interestingly, the 5′-end GGAAAA motif has been shown in some potexviruses, like AlsVX, LVX, ScaVX. While the possibility that these viruses show the unusual GGAAAA sequence in vivo cannot be completely excluded, the results above underlined that the finding of such a motif could be linked to the protocol used instead of representing accurately the genomic population found within the host and suggest that the consensus GAAAA motif could be a hallmark generally widespread within the potexviruses genus.

### Example 4

### Characterization of Malva Mosaic Virus by Sequence Analysis, Phylogenetic Analysis and Genomic Organization

The MaMV was further characterized as follows.

### Sequence Analysis

The MaMV sequence obtained as described in Example 2 was screened and compared against the BLASTn database at the National Center for Biotechnology Information (NCBI) (Altschul et al., 1997. Nucleic Acids Res. 25, 3389-3402.). Viral sequences were analyzed and assembled using the Contig Assembly Program (CAP) software (Huang, 1992. Genomics. 14, 18-25). Open reading frames (ORFs) were identified using NCBI ORF finder. Identity/similarity analysis were performed with the program GAP from the Wisconsin (GCG) package version 10.3, using a gap creation penalty of 8 and a gap extension penalty of 2 for amino acid comparisons (Anon, 2001. Wisconsin Package version 10.3. Accelrys Inc., San Diego, Calif., USA). The amino acid sequences of the replicase, TGB1 and capsid protein from various potexvirus strains were determined and entered into a multiple alignment generated by the Clustal W software (version 1.83) and corrected through final visual inspection with the SeqLab application (Wisconsin package version 10.3; Accelrys).

Excluding the poly(A) tail, MaMV genomic RNA is 6858 nucleotides (nt) long with a GC content of 45% (GenBank accession # DQ660333). The genomic organization is similar to other potexviruses, comprising a putative RNA-dependent RNA polymerase (RdRp), followed by three overlapping genes coding for the TGB proteins and finally, a coat protein (FIG. 6A; replicase is represented by the white box, triple gene block protein by the pale grey boxes and coat protein by the dark grey box. The black circle and diamond indicate the localization of the TGB1 and CP sgPromoter respectively (Abbreviations: MT: methyltransferase; A1 kB: DNA/RNA repair domain; HEL: helicase; POL: RNA-dependent RNA polymerase; CP: coat protein)). The suggested AUG initiation codon for the replicase is located at nucleotides 81-83 and translation from this site would produce a protein of 1571 amino acids (aa) for a calculated molecular weight of 177.96 kDa. The MaMV replicase derived from ORF1 is composed of at least three distinct domains (FIG. 6A), an N-terminal methyltransferase-like domain (aa 31-390), a NTP-binding/helicase-like domain (aa 824-1059) and a C-terminal RdRp2 domain (aa 1137-1535). These domains are generally well conserved and present within the replicase of all potexviruses (Rozanov et al., 1992. J. Gen. Virol. 73, 2129-2134; Koonin and Dolja, 1993. Crit. Rev. Biochem. Mol. Biol. 28, 375-430; Longstaff et al., 1993. EMBO J. 12, 379-386; Davenport and Baulcombe, 1997. J. Gen. Virol. 78, 1247-1251; Batten et al., 2003. Molecular Plant Pathology. 4, 125-131.). This analysis also highlighted the presence of a domain sharing homology to the DNA repair protein AlkB. A few other plant RNA viruses, mainly from the Flexiviridae family, harbour a similar motif within their replicase (Aravind and Koonin, 2001. Genome Biology. 2, research0007.1-0007.8; Bratlie and Drablos, 2005. BMC Genomics. 6, 1-15). As shown in FIG. 6A, the amino acid sequence and genomic organization of MaMV are similar to those found in other potexviruses.

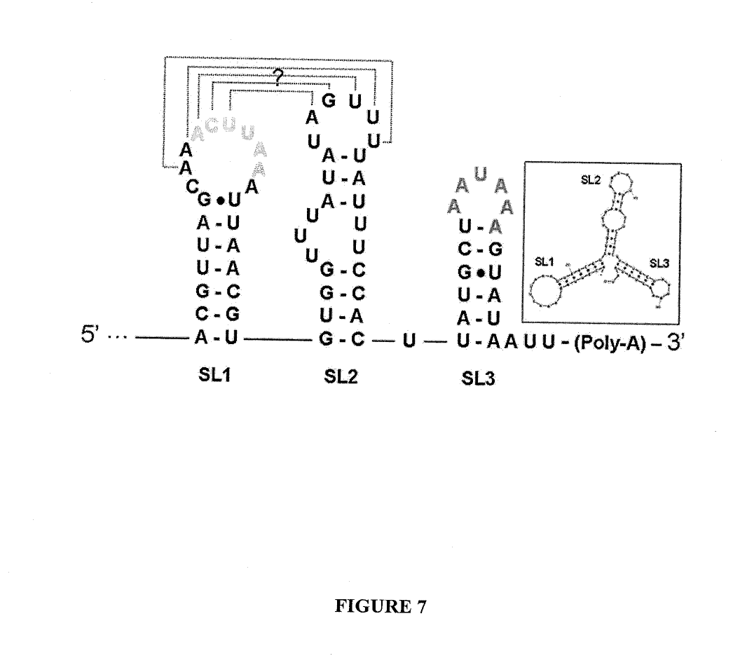

The next three ORFs are overlapping genes showing similarities with potexvirus TGB, which is involved in viral movement (Beck et al., 1991. Virology. 183, 695-702; Batten et al., 2003. supra; Morozov and Solovyev, 2003. J. Gen. Virol. 84, 1351-1366). TGB1 (ORF2) is a 235 aa protein of deduced molecular weight of 26.3 kDa. It has a very high content of leucine and charged residues (13.2% and 21.7% respectively). It is the most acidic TGB1 protein of all potexviruses with an isoelectric point (pI) of 4.84. Its sequence contains typical NTPase/helicase domains, which activity has been demonstrated in vitro for PVX and two hordeiviruses TGB1 proteins (Kalinina, N, O., et al., (2002) Virology 296(2):321-9). It is also suggested that TGB1 could play a role in inhibition of RNA silencing. The TGB2 protein (ORF3) is 119 aa long for a calculated molecular weight of 13 kDa and a theoretical pI of 9.42 while the ORF4, or TGB3, is a short protein of 84 aa (9 kDa) with a neutral pI (FIG. 7). In PVX, these two last proteins are associated with membranes and cell walls, and their functions are mainly to modulate TGB1 activity (Morozov et al., 1991. J. Gen. Virol. 72, 2039-2042; Yang et al., 2000. Mol. Plant-Microbe Interact. 13, 599-605; Morozov and Solovyev, 2003. supra). The conserved sequences and hydrophobic profiles of MaMV TGB proteins are typical to those of other potexvirus TGB proteins, suggesting that they could have similar activities.

The capsid or coat proteins (CPs) of potexviruses are involved in genome protection and virus movement. MaMV CP contained the conserved amphipathic core sequence KYAGFDFFDGVT (SEQ ID NO:20; encoded by nt 6545-6581), which is proposed to be responsible for binding of potexviruses RNA to the CP via hydrophobic interactions (Bancroft et al., 1991. J. Gen. Virol. 72, 2173-2181; Dolja et al., 1991. Virology. 184, 79-86; Wong et al., 1997. supra; Cotillon et al., 2002. Arch. Virol. 147, 2231-2238; Thompson and Jelkmann, 2004. Arch. Virol. 149, 1897-1909; Chen et al., 2005. Arch. Virol. 150, 825-832; Fuji et al., 2005. supra). The MaMV CP is a 243 aa protein (see FIG. 2 and SEQ ID NO:2), for a predicted molecular weight of 26 kDa.

A comparison of MaMV amino acid sequences with other potexviruses was performed and the results are summarized in Table 2 below. Viruses that are more closely related to MaMV are marked with an asterisk (*). Proteins with the highest homology with MaMV are highlighted in bold. Abbreviations and Genbank accession numbers: AlsVX: NC_{—}007408; BaMV: NC_{—}001642; CVX: NC_{—}002815; CsCMV: NC_{—}001658; ClYMV: NC_{—}001753; CymMV: NC_{—}001812; FoMV: NC_{—}001483; HdRSV: NC_{—}006943; LVX: NC_{—}007192; MVX; NC_{—}006948; NMV: NC_{—}001441; OVX: NC_{—}006060; PapMV: NC_{—}001748; PepMV: NC_{—}004067; PlAMV: NC_{—}003849; PAMV: NC_{—}003632; PVX: NC_{—}001455; ScaVX: NC_{—}003400; SMYEV: NC_{—}003794; TVX: NC_{—}004322; WClMV: NC_{—}003820; ZVX: NC_{—}006059.

TABLE 2
Amino acid identity/homology of MaMV with other potexviruses

| | Abbre- | | | | | |
|---|---|---|---|---|---|---|
| Virus | viation | Replicase | TGB1 | TGB2 | TGB3 | CP |
| *Alstroemeria | AlsVX | 64.2/ | 43.8/ | 42.7/ | 41.7/ | 67.1/ |
| virus X* | | 70.9 | 50.2 | 51.8 | 47.6 | 73.8 |
| Bamboo | BaMV | 44.4/ | 27.1/ | 45.0/ | 26.0/ | 22.8/ |
| mosaic virus | | 52.9 | 36.2 | 50.5 | 44.0 | 28.7 |
| Cactus virus X | CVX | 42.3/ | 35.1/ | 39.6/ | 28.1/ | 27.8/ |
| | | 51.6 | 43.7 | 47.2 | 40.6 | 35.0 |
| Cassava common | CsCMV | 44.2/ | 36.3/ | 36.8/ | 32.9/ | 29.8/ |
| mosaic virus | | 53.0 | 43.9 | 42.5 | 40.5 | 38.7 |
| Clover yellow | ClYMV | 45.8/ | 30.1/ | 33.0/ | 33.9/ | 30.3/ |
| mosaic virus | | 55.9 | 35.8 | 39.5 | 42.9 | 37.0 |
| Cymbidium | CymMV | 55.7/ | 39.4/ | 34.9/ | 39.2/ | 54.4/ |
| mosaic virus | | 64.4 | 45.7 | 45.9 | 55.7 | 63.1 |
| Foxtail | FoMV | 41.8/ | 27.9/ | 36.5/ | 35.6/ | 24.0/ |
| mosaic virus | | 52.3 | 35.4 | 45.9 | 44.4 | 32.2 |
| Hydrangea | HdRSV | 45.0/ | 29.6/ | 34.2/ | 27.8/ | 32.1/ |
| ringspot virus | | 53.5 | 39.8 | 39.6 | 30.6 | 38.0 |
| Lily virus X | LVX | 47.9/ | 33.2/ | 41.9/ | 21.5/ | 38.3/ |
| | | 57.6 | 42.0 | 45.7 | 30.8 | 47.3 |
| Mint virus X | MVX | 48.7/ | 35.4/ | 40.0/ | 30.8/ | 38.7/ |
| | | 58.5 | 45.0 | 47.6 | 37.2 | 46.2 |
| *Narcissus | NMV | 66.6/ | 52.8/ | 63.0/ | 53.6 | 75.6/ |
| mosaic virus* | | 72.8 | 60.9 | 69.7 | 66.7 | 81.3^{a} |
| Opuntia virus X | OVX | 43.7/ | 33.9/ | 39.0/ | 30.4/ | 30.1/ |
| | | 53.4 | 43.8 | 43.8 | 35.7 | 38.4 |
| Papaya mosaic | PapMV | 44.1/ | 30.0/ | 29.0/ | 28.6/ | 31.2/ |
| virus | | 54.0 | 36.6 | 36.4 | 41.3 | 37.2 |
| *Pepino | PepMV | 58.2/ | 37.0/ | 40.7/ | 33.8/ | 59.5/ |
| mosaic virus* | | 65.6 | 46.7 | 44.1 | 44.2 | 66.2 |
| Plantago asiatica | PlAMV | 45.8/ | 33.2/ | 45.8/ | 27.3/ | 31.1/ |
| mosaic virus | | 54.4 | 39.9 | 47.7 | 39.0 | 41.3 |
| Potato aucuba | PAMV | 50.3/ | 33.0/ | 44.4/ | 30.8/ | 43.8/ |
| mosaic virus | | 58.0 | 43.3 | 51.9 | 40.0 | 51.5 |
| Potato virus X | PVX | 44.4/ | 31.9/ | 42.6/ | 29.4/ | 31.8 |
| | | 53.8 | 36.4 | 50.0 | 39.7 | 40.7 |
| *Scallion | ScaVX | 67.2/ | 51.9/ | 60.5/ | 48.8/ | 71.3/ |
| virus X* | | 72.7 | 60.0 | 64.7 | 56.1 | 77.4 |
| Strawberry mild | SMYEV | 49.5/ | 33.6/ | 43.9/ | 34.5/ | 31.7/ |
| yellow edge virus | | 58.9 | 41.2 | 49.5 | 37.9 | 38.7 |
| Tulip virus X | TVX | 47.5/ | 32.9/ | 40.6/ | 25.7/ | 32.9/ |
| | | 55.9 | 40.9 | 42.5 | 32.4 | 39.6 |
| White clover | WClMV | 53.1/ | 35.4/ | 35.8/ | 27.9/ | 47.1/ |
| mosaic virus | | 61.3 | 44.5 | 40.4 | 42.6 | 51.9 |
| Zygocactus | ZVX | 42.7/ | 33.0/ | 38.5/ | 28.8/ | 33.0/ |
| virus X | | 52.9 | 43.0 | 43.1 | 37.3 | 40.2 |

^{a}The identity/homology results obtained for this protein have been obtained using the corrected NMV
coat protein sequence (see below).

This comparison revealed that ORF 1 (replicase) has the highest identity with the homologous protein from ScaVX while all the other ORFs (TGB1, 2 and 3, and coat protein) are more related to their NMV counterparts. PepMV and AlsVX also demonstrated good local homologies with MaMV, particularly in the replicase and capsid protein. However, AlsVX and PepMV seem globally farther from MaMV, mainly by reason of lower homology between their respective TGB proteins. As indicated above, the homology analysis revealed that the highest amino acid identity score between the replicase of MaMV and ScaVX (67.2%) and between the capsid protein of MaMV and NMV (75.6%). Since these values are lower than the molecular criteria of species demarcation established by Adams et al. (2004), supra, MaMV can be considered to be a different species from all previously published potexviruses.

Malva veinal necrosis potexvirus (MVNV) was reported in 1990 (Brunt et al., (eds.) 1996 onwards. “Plant Viruses Online: Descriptions and Lists from the VIDE Database. Version: 20 Aug. 1996”) in Brazil as a virus infecting Malva parviflora, on which local lesions and systemic veinal necrosis was detected. The nucleotide sequence of MVNV is not presently available. Although the original host from which MVNV was isolated is similar to the natural host for MaMV, the two virus are different for the following reasons: a) MaMV particles observed by electron microscopy are longer than MVNV, and b) MVNV was shown to induce local lesions on Malva species, while MaMV induces mosaic symptoms.

### Phylogenetic Analysis

Phylogenetic analyses were performed with MEGA version 3.1 (Kumar et al., 2004. Brief Bioinform. 5(2), 150-163) using distance methods and the neighbour-joining algorithm. The topological accuracy of the tree was evaluated using 500 bootstrap replicates. The 5′ and 3′ untranslated region (UTR) structural analyses were performed using the program mfold version 3.2 (Zuker, 2003. Nucleic Acids Res. 31, 3406-3415). Multiple alignments and profile of protein domains were performed with the Pfam database of protein families (version 18) located at the Wellcome Trust Sanger Institute at Cambridge website using the default parameters (Bateman et al., 2004. Nucleic Acids Res. 32, 138-141). The multiple alignment underlined a frameshift error in the C-terminal region of the NMV CP (NC_{—}001441) which indicates that the last 45 aa of this protein is completely unrelated to any other potexviruses. The corrected NMV sequence was therefore used for the homology/phylogenetic analysis.

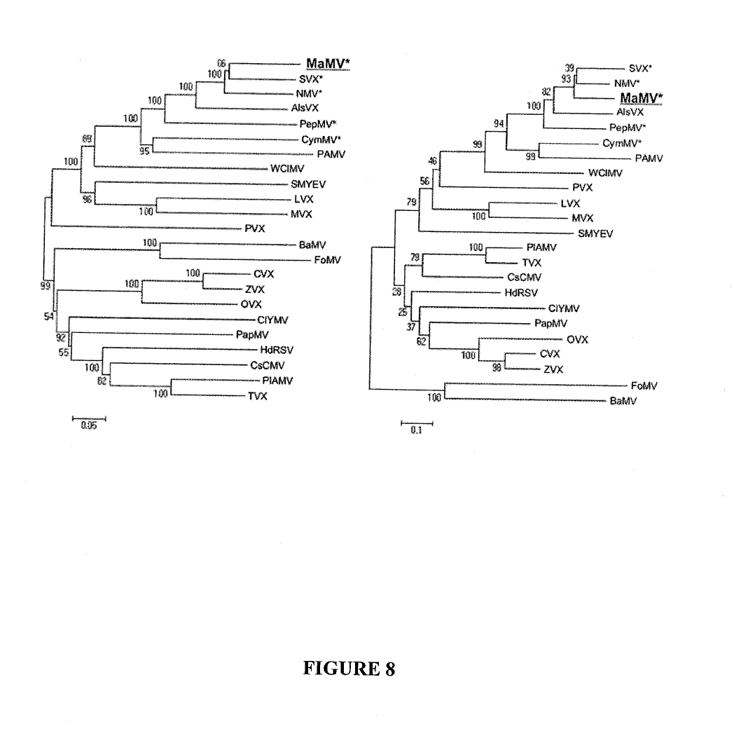

Phylogenetic analyses were performed in order to study more accurately the relationship between MaMV and other potexviruses. The analysis was performed using the most conserved region of the replicase and capsid amino acid sequences (FIG. 8) as well as the complete sequence of TGB1 proteins. FIG. 8 depicts the phylogenetic analysis of the replicase and capsid proteins of MaMV and other potexviruses. The phylogenetic tree of the replicase and the coat protein are at the left and right panel respectively. Numbers indicate the bootstrap value of each branch (500 replications). Viruses highlighted with an asterisk possess a similar predicted pseudoknot structure as identified at the 3′-end untranslated region of MaMV.

The phylogenetic analysis revealed that the conserved sequences usually detected in potexviruses are found within MaMV. The phylogenetic analysis supports the previous observation that MaMV appeared to be most closely related to a subgroup of potexviruses comprising NMV, ScaVX, AlsVX and PepMV, the latter two being slightly more phylogenetically distant. In every tree analyzed, MaMV, NMV and ScaVX were always grouped together, again supporting this close relationship. However, the low bootstrap value observed within this subgroup for the replicase and the capsid protein (66% and 39% respectively), did not allow an unequivocal conclusion as to which of these two viruses MaMV is most closely related. Phylogenetic analysis performed with the TGB1 gave similar results and also did not allow a conclusion as to the closest relative to MaMV.

### Subgenomic Promoters and Analysis of Conserved RNA Promoter Elements

Total RNA was extracted from healthy and MaMV-infected C. quinoa plants as follows. Infected leaves and non-infected leaves of C. quinoa were harvested and ground in liquid nitrogen. Total RNA was extracted using phenol/chloroform and further purified using a RNA spin column (Qiagen). Total RNA was analyzed by Northern blotting as follows: RNA (20 μg) was separated on a formaldehyde agarose gel (1%) and then transferred to a nylon membrane (Amersham Biosciences). The immobilized RNA was probed with a cDNA fragment of the MaMV coat protein labeled by PCR with digoxigenin (Roche Diagnostics). Hybridization was performed according to the manufacturer's protocol. The hybridization signals were visualized with chemiluminescent substrate CDP-Star (Roche Diagnostics).

The Northern blot revealed that 3 major viral RNAs were produced during infection, one large genomic RNA, and two subgenomic species migrating approximately as a RNA of 2000 and 800 nucleotides respectively (FIG. 6B). The subgenomic species should correspond to the viral subgenomic RNAs encoding for the triple gene block and the viral CP (2000 nt long RNA) and the viral CP alone (800 nt RNA). The identification of sgpromoter consensus sequences in MaMV strongly suggests that the TGB and the CP are translated from their own sgRNA in vivo. However, the possibility that they can be produced by internal initiation on the genomic RNA is not excluded since this mechanism has been previously observed, at least for PVX CP (Hefferon et al., 1997. J. Gen. Virol. 78, 3051-3059).

Octanucleotide subgenomic promoter sequences have been found in the intergenic region between the replicase and TGB1 gene (nt 4851-4858) and between nt 5982-5989 in the 3′-end of TGB3 coding sequence. FIG. 6C shows the sequence alignment of the octanucleotide putative sgPromoter sequence. Left and right hand panels represent the TGB (black circle) and CP (black diamond) sgPromoter consensus sequence respectively. Highly conserved nucleotides within the consensus octanucleotide are highlighted with black or grey boxes respectively. Nucleotides that differ from the consensus sequence are in white.

Both promoters have the exact consensus sequence GTTAAGTT retrieved in most potexviruses (Skryabin et al., 1988. FEBS. 240, 33-40; Kim and Hemenway, 1997. Virology. 232, 187-197; Batten et al., 2003. supra;). The TGB2/TBG3 sgpromoter consensus sequences reported for PVX are not as well defined as those of TGB1 and CP and the precise identification of such domain is consequently more ambiguous (Skryabin et al., 1988. supra). sgRNA that correspond to species that would correlate with active TGB2/TGB3 promoters were not detected in these infected plants. If these sgRNA species exist, it is likely that they are not as abundant as TGB1 and CP sgRNAs.

The 3′ UTR is 70 nt long and is predicted to fold into a tRNA-like secondary structure, similar to those identified in other potexviruses (Thompson and Jelkmann, 2004. supra). The 3′ UTR contains the polyadenylation signal AAUAAA 14 nt upstream of the polyadenylation site as well as the conserved hexamer ACUUAA, present in all potexviruses sequenced to date (nt 6799-6804). Both sequences are localized into a distinct loop of the three stem-loop structures (SL3 and SL1 respectively) (FIG. 7). FIG. 7 is a schematic representation of the secondary structure of the MaMV 3′-untranslated region. The 3 stem-loop structures are identified as SL1, SL2 and SL3. The consensus sequence ACUUAA found in all potexviruses is highlighted by the pale grey nucleotide in SL1 while the polyadenylation signal located in SL3 is in dark grey. The secondary structure obtained by the program mfold is represented within the inset. The putative novel pseudoknot between SL1 and SL2 is indicated by the dashed line. The question mark (?) indicates that the pseudoknot is speculative and that the folding of this structure in solution has yet to be determined.

This analysis revealed a putative 5-base pair pseudoknot between nucleotides located in loop of SL1 and SL2. A similar structure has been previously described for PVX but in this case, the complementarity region was between nucleotides from the subgenomic promoter and the ACUUAA conserved sequence of SL1 (Kim and Hemenway, 1997. supra). Analysis of the 3′-end of other potexviruses revealed that similar pseudoknots can be detected in some other viruses like NMV, CymMV, ScaVX, and PepMV. However, the complementary sequence forming the pseudoknot in the last two viruses is only 4 nucleotides long.

### Example 5

### Malva Mosaic Virus Coat Protein Production, Purification and Self-Assembly in E. coli

The complete MaMV coat protein gene was amplified by PCR from a sequencing plasmid encompassing the entire 3′-end of the genomic RNA (containing the sequences encoding the CP and the triple gene block; see Example 2) using the forward primer (5′-GGTACATGTCGAACTCTGGTTCAGCCG-3′, SEQ ID NO:21) and reverse primer (5′-TACGGATCCTCAATGGTGATGGTGATGGTGGAATTCTGGGGGGGCTTCAA TGG-3′, SEQ ID NO:22). The forward primer contained an Afl III restriction site (underlined), which included the initiation codon for the cloning of the PCR product into the pET-3D expression vector, while the reverse primer allowed the addition of a 6×-His tag (underlined) to the 3′-end of the CP gene for further purification of the protein on a nickel affinity column. The PCR reaction was performed as follows: 45 s at 94° C., 45 s at 65° C. and 60 s at 72° C. for 33 cycles with a pre-incubation of 3 min at 94° C. using the Expand™ High Fidelity PCR system (Roche Diagnostics) under standard conditions recommended by the manufacturer. The PCR products were digested by Afl III/Bam H1 restriction enzyme and ligated into the pET-3D compatible Nco I/Bam H1 restriction sites. Ligation products were transformed into and amplified in E. coli DH5α and the plasmid DNA extracted and sequenced as described in Example 2.

A plasmid identified as containing the CP gene (pMaMV-CP-6H) was used to transform the E. coli strain BL21 (DE3) RIL cells (Invitrogen). The transformed cells were spread on 2×YT agar plates with 50 μg/ml of ampicillin and incubated overnight at 37° C. Culture media (2×YT, 50 μg/ml ampicillin) was inoculated from a pre-culture of about 10 isolated colonies and grown until an OD of 0.6-0.8 was reached. Protein expression was induced by the addition of IPTG (Promega) to a final concentration of 1 mM and the culture incubated at 22° C. for 16 hours with shaking at 225 RPM. The cells were then centrifuged and resuspended in lysis buffer (50 mM NaH₂PO₄, 300 mM NaCl, 10 mM imidazole pH 8.0) in presence of 20 μM PMSF (EM Science), 1× protease cocktail inhibitor (Roche) and 1 mg/ml lysozyme (Sigma) before being sonicated on ice with a Sonic Dismembrator model 500 sonicator (Fisher Scientific). The protein solution was centrifuged and the supernatant was incubated for 3 hours at 4° C. in the presence of 2.5 ml of Nickel-NTA agarose beads before being poured into an elution column (Bio Rad). Beads were washed with 25 ml of each of the following buffers: washing buffer 1 (50 mM NaH₂PO4, 300 mM NaCl, 20 mM imidazole, pH 8.0), washing buffer 2 (50 mM NaH₂PO₄, 300 mM NaCl, 50 mM imidazole, pH 8.0) and washing buffer 3 (10 mM Tris-HCl, 50 mM imidazole, pH 8.0). The beads were then incubated 30 min in presence of elution buffer (10 mM Tris-HCl, 1M imidazole, pH 8.0) to elute the protein. The resulting eluted protein was analyzed by 10% SDS-PAGE and by electron microscopy as described above to check for the formation of virus like particles (VLP).

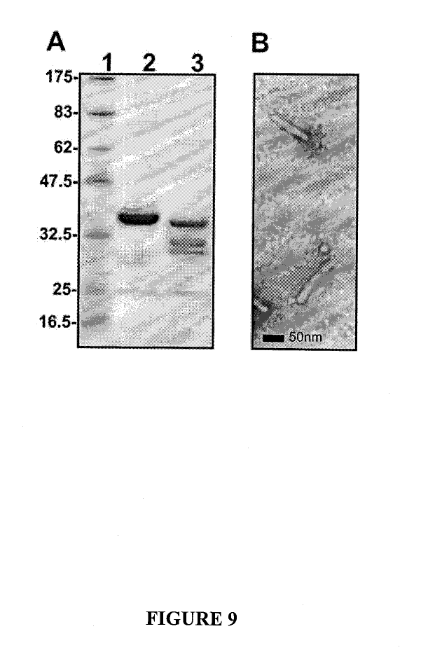

Using SDS-PAGE analysis of the protein extracts, a predominant form of 34-35 kDa was detected (FIG. 9A; Lane 1: Protein molecular weight marker. Molecular weight markers are shown on the left in kDa; Lane 2: Purified coat protein produced in E. coli; Lane 3: Coat protein isolated from purified MaMV from infected plants) The mobility of the coat protein on the polyacrylamide gel is consistent with other potexviruses coat proteins (Tremblay et al, 2006. FEBS. 273, 14-25; Hu and Ghabrial 1995, J. Virol. Meth. 55, 367-379; Hammond and Hull, 1981. J. Gen. Virol. 54, 75-90). A slight difference in the molecular weight of MaMV CP produced in E. coli compared to the wild type protein can be explained by the additional 6×-His tag (FIG. 9A).

Finally, the purified recombinant protein was observed using electron microscopy which confirmed that overexpression of the MaMV CP resulted in self assembly of the coat protein in E. coli into virus like particles that are very similar to the native virus (see FIG. 9B, which shows an electron micrograph of the virus-like particles made of the purified recombinant MaMV CP (Bar is 50 nm long).

The nucleotide sequence of the MaMV coat protein gene contained in plasmid pMaMV-CP-6H is provided in FIG. 10 (SEQ ID NO: 23) and the amino acid sequence of the encoded coat protein is provided in FIG. 11 (SEQ ID NO: 24).

### Example 6

### Analysis of Purified Recombinant Malva Mosaic Virus Coat Protein by LC-MS/MS Analysis

The purified recombinant coat protein from Example 5 was analysed by LC-MS/MS analysis (Eastern Quebec Proteomics Centre, Centre Hospitalier de l'Université Laval, Québec). The sample was lyophilized before reduction and alkylation with 45 mM dithiothreitol and 100 mM iodoacetamide in 50 mM ammonium bicarbonate buffer. After dilution in acetonitrile, tryptic digestion was performed at 37° C. overnight using 0.2 μg of sequencing grade modified trypsin (Promega). Digestion was stopped using formic acid and 2 μL of the sample was injected in the mass spectrometer. Peptide MS/MS spectra were obtained by capillary liquid chromatography coupled to an LTQ (Thermo-Electron, San Jose, Calif., USA) quadrupole IT mass spectrometer with a nanospray interface. Chromatographic separation was achieved on a PicoFrit column BioBasic C18, 10 cm×75 μm, (New Objective, Woburn, Mass.) with a linear gradient from 2-50% solvent B (acetonitrile, 0.1% formic acid) in 30 minutes, at 200 nL/min Peptides eluted through the column directly into the LTQ linear ion trap mass spectrometer with the spray voltage set to 1.8 kV and the transfer capillary temperature set to 225° C. Mass spectra acquisition was controlled by Xcalibur 2.0 SR 2 software (ThermoElectron Corp.) using a data dependent acquisition mode in which each full scan mass spectrum (400 to 2000 m/z) was followed by collision-induced dissociation of the seven most intense ions. The dynamic exclusion function was enabled, and the relative collisional fragmentation energy was set to 35%.

Resulting MS/MS spectra were interpreted using MASCOT (Matrix Science, London, UK; version 2.2.0) and searched against 3 different databases: one containing the protein of interest, Uniref Human databank and Uniref E. coli database (version 8.0, containing respectively 94 985 and 40 567 entries). Carbamidomethylation of cysteine and partial oxidation of methionine, two missed cleavages, and an error tolerance of 2.0 Da for peptides and 0.5 Da for fragments were considered in the searches. Scaffold (version Scaffold-01_{—}06_{—}18, Proteome Software Inc., Portland, Oreg.) was used to validate MS/MS based peptide and protein identifications. Peptide identifications were accepted if they could be established at greater than 90.0% probability as specified by the Peptide Prophet algorithm (Keller, A et al. 2002; Anal. Chem. 74(20):5383-92). Protein identifications were accepted if they could be established at greater than 90.0% probability and contained at least 2 identified peptides. Protein probabilities were assigned by the Protein Prophet algorithm (Nesvizhskii, A. I., 2003, Anal Chem. 75(17):4646-58). Proteins that contained similar peptides and could not be differentiated based on MS/MS analysis alone were grouped to satisfy the principles of parsimony.

The digestion of the purified recombinant protein with trypsin generated 30 peptides that were analysed by LC-MS/MS which confirmed that all the peptides belong to the MaMV CP sequence. The MaMV CP has a molecular weight of 25,961.2 Da. The 30 peptides that were generated had a mass that corresponded 100% to the expected fragment of the MaMV protein. The peptides covered 93% of the amino acid sequence of the MaMV CP, confirming the identity of the protein that was purified and overexpressed in E. coli. Table 3 shows the LC-MS/MS analysis of the MaMV coat protein peptides generated from trypsin digestion (SEQ ID NOs:25-53).

TABLE 3
LC-MS/MS analysis of the
MaMV coat protein peptides

| | Peptide | Peptide | Peptide | SEQ |
|---|---|---|---|---|
| Peptide sequence | mass | start | stop | ID NO |
| AINYVSTTTAVATPAEIK | 1849.9913 | 48 | 65 | 25 |
| AINYVSTTTAVATPAEIK | 2664.4614 | 48 | 72 | 26 |
| LLGDLFR | | | | |
| APTLEDLK | 886.4888 | 40 | 47 | 27 |
| APTLEDLKAINYVSTTTA | 2717.4609 | 40 | 65 | 28 |
| VATPAEIK | | | | |
| AYADVQASR | 980.4800 | 91 | 99 | 29 |
| AYADVQASRSAILSGSTP | 2549.2956 | 91 | 115 | 30 |
| SNPSITR | | | | |
| EIQAHSTAK | 984.5114 | 194 | 202 | 31 |
| GIDANAVAPAMWDLAR | 1670.8326 | 75 | 90 | 32 |
| GIDANAVAPAMWDLARAY | 4201.1099 | 75 | 115 | 33 |
| ADVQASRSAILSGSTPSN | | | | |
| PSITR | | | | |
| IQNGNYVSNLAEVTHGR | 1871.9364 | 211 | 227 | 34 |
| IVWNMMLATHTPPANWAK | 2081.0466 | 139 | 156 | 35 |
| KGIDANAVAPAMWDLAR | 1798.9277 | 74 | 90 | 36 |
| KKGIDANAVAPAMWDLAR | 1927.0227 | 73 | 90 | 37 |
| KPAENIPSQEPQPADPAD | 2258.1057 | 19 | 39 | 38 |
| PTR | | | | |
| KPAENIPSQEPQPADPAD | 3125.5759 | 19 | 47 | 39 |
| PTRAPTLEDLK | | | | |
| LLGDLFR | 833.4887 | 66 | 72 | 40 |
| LLGDLFRK | 961.5837 | 66 | 73 | 41 |
| MPNQK | 617.3082 | 189 | 193 | 42 |
| MPNQKEIQAHSTAK | 1582.8013 | 189 | 202 | 43 |
| MSNSGSAAAAPSQPSAAK | 1632.7651 | 1 | 18 | 44 |
| QFCMYFAK | 1094.4805 | 131 | 138 | 45 |
| QGLPEDCK | 946.4306 | 157 | 164 | 46 |
| QGLPEDCKYAGFDFFEGV | 3498.6890 | 157 | 188 | 47 |
| LSPSALEPADGLIR | | | | |
| QLYSIDLTPR | 1205.6532 | 121 | 130 | 48 |
| QRIQNGNYVSNLAEVTHGR | 2156.0959 | 209 | 227 | 49 |
| SAILSGSTPSNPSITR | 1587.8343 | 100 | 115 | 50 |
| SNSGSAAAAPSQPSAAK | 1501.7246 | 2 | 18 | 51 |
| TALAK | 503.3194 | 116 | 120 | 52 |
| YAGFDFFEGVLSPSALEP | 2571.2771 | 165 | 188 | 53 |
| ADGLIR | | | | |

### Example 7

### Immunogenic Effect of Malva Mosaic Virus VLPs

The MaMV coat protein gene was expressed in E. coli BL21 (pLysS) from the plasmid pMaMV-CP-6H as follows: A 1 L culture of E. coli BL21 (pLysS) containing pMaMV-CP-6H was prepared and expression of the encoded coat protein was induced with 1 mM IPTG. Expression was allowed to proceed O/N at 25° C. The cells were harvested and lysed using a French press at 750 psi (lysis buffer: 50 mM NaP buffer pH8, 20 mM imidazole and 300 mM NaCl). Debris was removed by 2× centrifugation at 10,000 g for 45 min and 30 min. respectively, and the supernatant retained. 12 ml Ni²⁺ beads (Qiagen) were added per 1 L of bacterial culture and incubated in batch O/N at 4° C. with gentle shaking. The beads containing the bound protein were placed in a econo-column and washed with (a) 50 ml washing buffer (10 mM Tris-HCl pH8+50 mM imidazole), followed by (b) 50 ml washing buffer+0.5% Triton X-100, (c) 50 ml washing buffer, (d) 50 ml washing buffer plus 1% Zwittergent, and (e) 50 ml washing buffer. The protein was then eluted with 1M imidazole.

Eluted protein was dialysed against 3 L of 10 mM Tris-HCl pH 8 for an hour. The buffer was exchanged for fresh and the dialysis continued for a further hour. A final change of buffer was performed and the dialysis continued O/N. The protein was subsequently dialysed against 3 L of PBS, with 3 changes of buffer at 20 minute intervals. The concentration of NaCl was subsequently increased to 500 mM and the suspension centrifuged at high speed (100,000 g) for 3 hours to pellet the VLPs.

The VLP pellet was resuspended in sterile PBS and filtered through a 0.45 micron filter. Finally, the concentration of the protein was adjusted to 1 mg/ml. The presence of VLPs was confirmed using the Electron microscope, and the presence of any LPS contaminant was determined using the Limulus test.

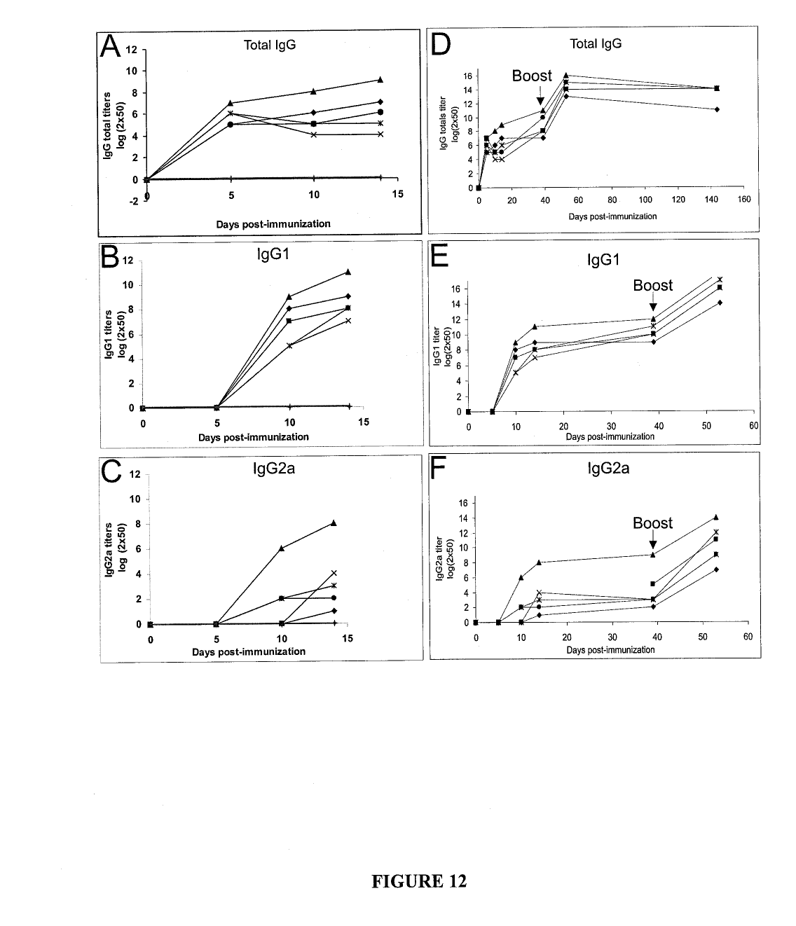

The immunogenicity and ability of the MaMV VLPs to elicit an immune response was tested in mice following standard protocols. Briefly, five Balb/C mice were injected subcutaneously at day 0 with 100 μg of MaMV VLPs without adjuvant. Blood samples were collected at days 5, 10 and 14 after immunisation. The total amount of IgG, the amount of Ig1 and the amount of IgG2a directed toward the VLPs present in the sera were measured by standard ELISA (see FIG. 12A-C).

The same mice were immunised a second time at day 40 with 100 μg of MaMV VLPs s.c. without adjuvant. The total amount of IgG, the amount of IgG1 and the amount of IgG2a directed toward the VLPs present in the sera were again measured by standard ELISA (see FIG. 12D-F).

The results show high levels of IgG and IgG1, which indicates that MaMV VLPs are highly immunogenic and can trigger an efficient antibody response. Furthermore, the presence of IgG2a suggests activation of CD4+ T cells and an efficient induction of the antibody class switch. This result suggests that a balanced TH1 and TH2 response is induced.

### Example 8

### Purification of Salmonella typhi Porin Proteins

The following purification procedure was used for purification of Salmonella typhi OmpC and OmpF proteins suitable for use as antigens in conjunction with MaMV or MaMV VLPs. The purification procedure is based on that described by Secundino et al. (2006), Immunology 117:59.

The two proteins were co-purified from Salmonella typhi. Individual purification of OmpC and OmpF was achieved using knock-out mutants of S. typhi in which either OmpC [STYC171 (OmpC)] or OmpF [STYF302 (OmpF⁻)] open reading frames are interrupted. The procedure for purification of the individual proteins from the knock-out mutated forms of the bacteria was followed as for the co-purification. This procedure is outlined below.

The bacterial strain, Salmonella typhi 9,12,Vi:d (ATCC 9993) was grown in Minimal medium A supplemented with yeast extract, magnesium and glucose at 37° C., 200 rpm. The formula for 10 L Minimal medium A supplemented with yeast extract, magnesium and glucose is: 5.0 g of dehydrated Na-Citrate (NaC₆H₅O₇:2H₂O), 31.0 g NaPO₄ monobasic (NaH₂PO₄), 70.0 g NaPO₄ dibasic (Na₂HPO₄), 10.0 g (NH₄)₂SO₄, 200 mL yeast extract solution 5% (15.0 g in 300 mL). 1.434 L medium was distributed per 4 L Erlenmeyer flask. Sterilization was performed at 121° C., 15 lbs pression/in², 15 min. o each flask was then added: 6.0 mL of sterile MgSO4 solution 25% and 60.0 mL of glucose solution 12.5%. The flask was inoculated with an overnight culture of S. typhi and when the OD₅₄₀ reached 1.0, incubation was stopped and the culture centrifuged at 7,500 rpm for 15 min at 4° C. The pellet was resuspended in 100 mL final of Tris-HCl pH 7.7 (6.0 g Tris-base/L) and the biomass was sonicated for 90 min on ice and then centrifuged at 7,500 rpm for 20 min at 4° C. To each 10 mL of supernatant was added: 2.77 mL MgCl₂ 1M, 25 ml RNaseA (10,000 U/mL), 25 ml DNaseA (10,000 U/mL). The mixture was then incubated at 37° C. and 120 rpm for 30 min.

Porin extraction from the mixture was performed as follows:
1. Ultracentrifugation was performed at 45,000 rpm, 45 min, 4° C. and the pellet retained.
2. The pellet was resuspended in 10 mL Tris-HCl-SDS 2% followed by homogenisation.
3. An incubation step was performed at 32° C., 120 rpm, 30 min.
4. Ultr acentrifugation followed at 40,000 rpm, 30 min, 20° C. and the pellet retained.
5. The pellet was resuspended in 5 mL Tris-HCl-SDS 2% followed by homogenisation.
6. An incubation step was performed at 32° C., 120 rpm, 30 min.
7. Ultr acentrifugation followed at 40,000 rpm, 30 min, 20° C. and the pellet retained.
8. The pellet was resuspended in 20 mL Nikaido buffer-SDS 1% followed by homogenisation. [For 1 L of Nikaido buffer: 6.0 g Tris-base, 10.0 g SDS, 23.4 g NaCl, 1.9 g EDTA was dissolved in water and the pH adjusted to pH 7.7. 0.5 mL β-mercaptoethanol solution was then added]
9. The mixture was incubated at 37° C., 120 rpm, 120 min.
10. Ultracentrifugation followed at 40,000 rpm, 45 min, 20° C. The supernatant, which contained the porin extract, was recovered.

The porins were purified from the supernatant using fast protein liquid chromatography (FPLC). 0.5× Nikaido buffer (see above) without β-mercaptoethanol was employed during the purification process. The proteins were separated using a Sephacryl S-200 (FPLC WATERS 650 E) with a Flux speed: 10 mL/min. The column was loaded with 22 mL of supernatant. Eluted fractions were monitored at 260 and 280 nm. The main peak, which contained the purified porins, was retained and stored at 4° C. The purified porins were stable for long period (over one year).

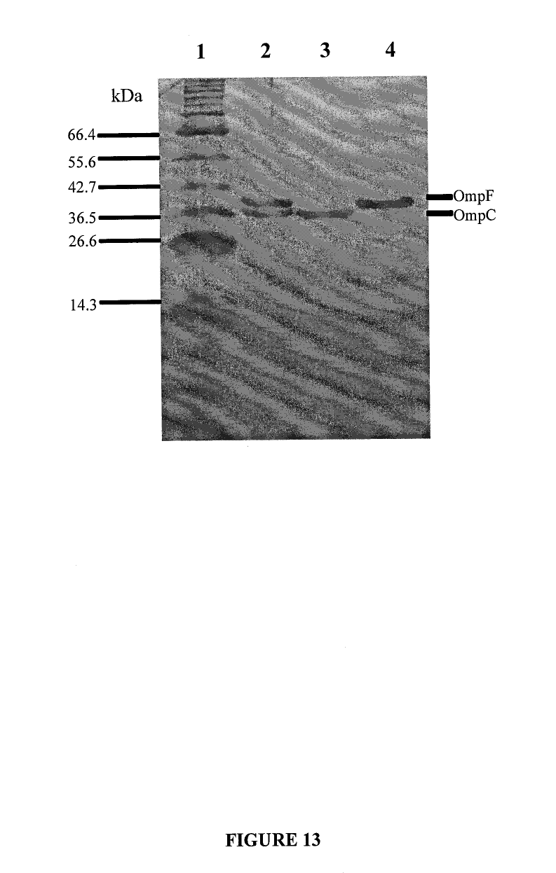

FIG. 13 shows the SDS-PAGE profile of the porins, OmpC and OmpF, purified by the procedure described above.

### Example 9

### Production of Affinity Peptides Suitable for Attachment of Salmonella typhi Porins to Malva Mosaic Virus VLPs

Specific peptides against purified OmpC and OmpF were selected using the Ph.D-7 Phage Display Peptide Library Kit (New England Biolabs, Inc.). The protocol followed was an in vitro selection process known as “panning,” which was conducted according to the manufacturer's protocol. Briefly, 2×10¹¹ phage were added to 10 ug of purified OmpC or OmpF bound to the base of the wells of an ELISA plate and the contents of the well gently mixed at room temperature for 1 hour. Unbound phage were eluted with 1 ml of 200 mM Glycine-HCl (pH 2.2), by incubating for 10 min at room temperature. To neutralize the supernatant, and to avoid killing the phage, 150 μl of 1M Tris-HCl (pH 9.1) was added. The eluted phage were then amplified and taken through additional binding/amplification cycles to enrich the pool in favour of binding sequences. The wash buffer contained 0.1% of Tween 20 for the first round of panning and was increased to 0.5% for subsequent rounds. Selected phage were amplified in E. coli ER2738 between each panning round. The cycle was repeated 3 times to select those peptides with the highest affinity for the respective porin proteins. The peptides thus identified are shown in Table 4.

TABLE 4
Sequence and Frequency of Occurrence
of OmpC and OmpF Affinity Peptides

| | Sequence | | SEQ |
|---|---|---|---|
| Target | of | | ID |
| Protein | Peptide | Frequency | NO |
| OmpC | SLSLIQT | 1/8 | 54 |
| OmpC | EAKGLIR | 6/8 | 55 |
| OmpC | TATYLLD | 1/8 | 56 |
| OmpF | FHENWPS | 3/5 | 57 |
| OmpF | FHEFWPT | 2/5 | 58 |

The affinity peptides identified above can be engineered into the C-terminus of the MaMV coat protein, for example, by PCR and the resulting fusion protein can be expressed in E. coli and VLPs from the fusion protein will be produced. The VLPs can then be mixed with their cognate porin in solution, for example in a 1:1 ratio to provide a complex comprising the VLP and the porin. The complex, either alone or in combination with additional porin, can subsequently be used to immunize mice against challenge with S. typhi.

### Example 10

### Adjuvant Effect of Malva Mosaic Virus VLPs on a Commercial Vaccine

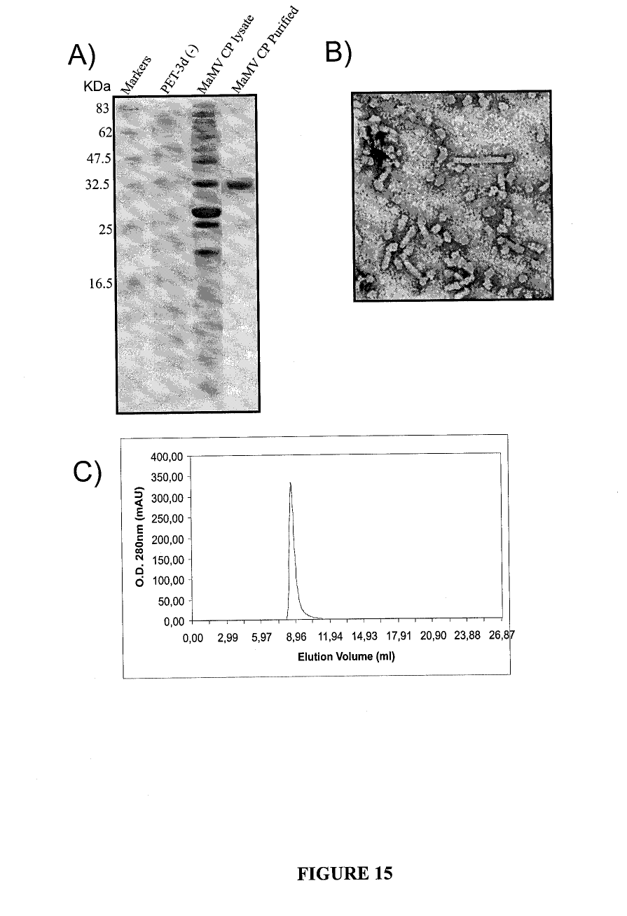

Recombinant MaMV CP having a sequence as set forth in SEQ ID NO:24 (see FIG. 11A) was over-expressed in E. coli at 22° C. for 16-22 hours. The bacteria were lysed using a French press, the sample centrifuged to remove the debris, and loaded on a Ni²⁺ column for affinity purification. The coat proteins purified by affinity chromatography are shown in FIG. 15A. The VLPs formed from these CPs have different lengths, but are similar to the WT virus (FIG. 15B) and were excluded from a Superdex 200 gel filtration column showing that all protein was found in high molecular weight form (VLPs more than 500 kDa) since the monomeric or lower multimeric form (less than 500 kDa) were not detected (FIG. 15C). The LPS levels in the MaMV VLP preparation were evaluated at 24.0 EU/mg. In subsequent immunisations (see below), 3 or 30 μg of the VLPs were used and for these amounts, LPS contamination is considered to be negligible.

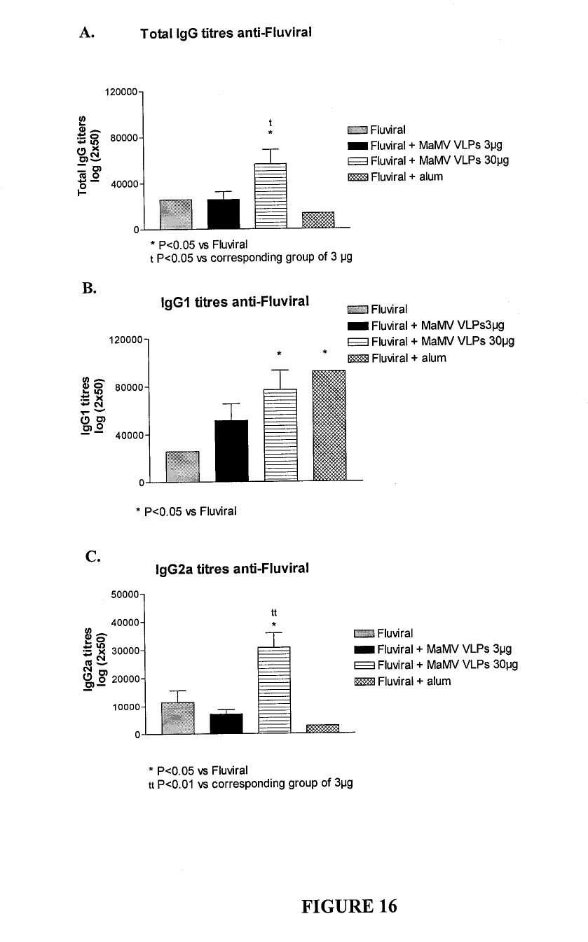

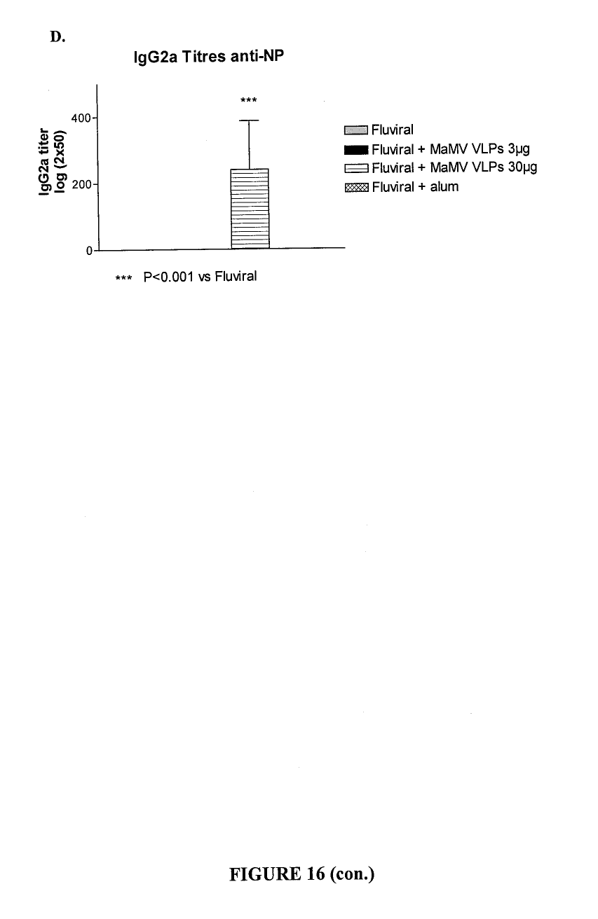

To measure the adjuvant property of the MaMV VLPs, a trivalent flu vaccine Fluviral® (GlaxoSmithKline) as a model system. One-fifth of the human dose of Fluviral® was adjuvanted with either 3 μg or 30 μg of MaMV VLPs or with the conventional adjuvant, alum, and administered sub-cutaneously to Balb/C mice, 5 per group. Bleeding was done at day 14 after immunization and the immune response to Fluviral® or the purified influenza NP (nucleocapsid of influenza WSN/33) protein were performed by ELISA. Total IgG (FIG. 16A), IgG1 (FIG. 16B), IgG2a (FIG. 16C) to Fluviral® were measured. IgG2a to NP were also measured (FIG. 16D).

The results shown in FIG. 16 indicate that 30 μg of MaMV VLPs could significantly improve the immune response to Fluviral®. A significant increment of total IgG (FIG. 16A), IgG1 (FIG. 16B) and IgG2a (FIG. 16C) were observed against the Fluviral® proteins, while alum failed to improve significantly the amount of total IgG and IgG2a. MaMV VLPs also induced a T_{H2} immune response (IgG1) and show a similar efficacy to alum in this regard (FIG. 16B). In addition, MaMV VLPs were shown to induce the production of IgG2a directed to the NP protein (FIG. 16C). Fluviral® and Fluviral® adjuvanted with alum were unable to induce production of any IgG2a to the NP protein. This result suggests that the NP protein, which is found in the interior of the influenza virus and is present in the Fluviral® preparation, became immunogenic only in presence of the MaMV VLPs. This result also suggests that a T_{H1} response was induced since the class switch leads to production of large amount of the IgG2a isotype, which is an ideal response for protection against a viral infection, and demonstrates that MaMV VLPs have excellent adjuvant properties.

The NP protein is one of the most conserved protein in all the strains of influenza (more than 92% identity). As such, it is expected that the induction of the immune response to this protein, or any other conserved epitope found in the Fluviral® vaccine, using the MaMV-based adjuvant, will provide a protection to strains of influenza that are unrelated to the those found in the commercial vaccine, i.e. the addition of the MaMV-based adjuvant to commercially available influenza vaccine can potentially provide a formulation capable of providing protection to multiple strains of influenza.

### Example 11

### Production and Testing of Malva Mosaic Virus VLPs Comprising Coat Protein Genetically Fused to an Antigen

The following Example demonstrates that MaMV VLPs can be used as a vaccine platform. All coat proteins described in this Example were purified using the same CP purification procedure as described in the preceding Examples.

The C-terminus of the MaMV coat protein was engineered to include SpeI and MluI restriction sites to ease the cloning of small annealed oligonucleotide encoding an appropriate antigen directly at the C-terminus of the coat protein. The sequence 5′ ACTAGTACGCGT 3′ (SEQ ID NO:61) containing a SpeI/MluI site was be cloned into the coat protein gene shown in FIG. 10 in the position just after the last amino acid (phenylalanine: F) and before the 6×H tag. The recognition sequences of each of the enzymes are as follows:

### SpeI=ACTAGT

### MluI=ACGCGT

The resulting MaMV CP is named MaMV CP-SM. The nucleotide sequence encoding this MaMV CP-SM is shown in FIG. 10B (SEQ ID NO:62) and the amino acid sequence of the MaMV CP-SM protein is shown in FIG. 11B (SEQ ID NO:63).

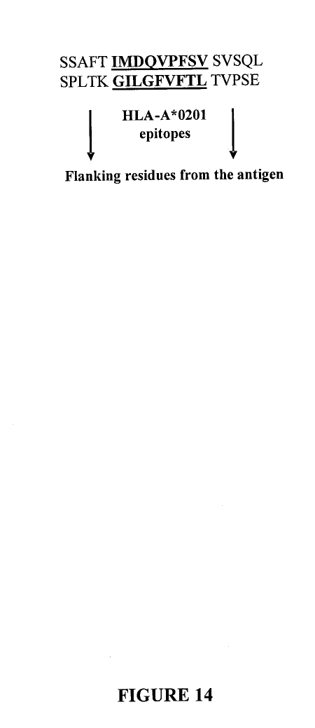

9-mer HLA-A*0201 epitopes from the well-defined tumor antigen gp100 (IMDQVPFSV; SEQ ID NO:59), and from influenza M1 protein (GILGFVFTL; SEQ ID NO:60) were chosen for fusion with the MaMV CP. The HLA-A*0201 epitopes were flanked on the N- and C-terminal sides by 5 residues from the respective native sequences to favour natural processing by the proteasome (see FIG. 14). Upon fusion with the Influenza or the gp100 epitope, the amino acids TS and TR will be fused to the N terminus and the C terminus, respectively, of the fused epitope.

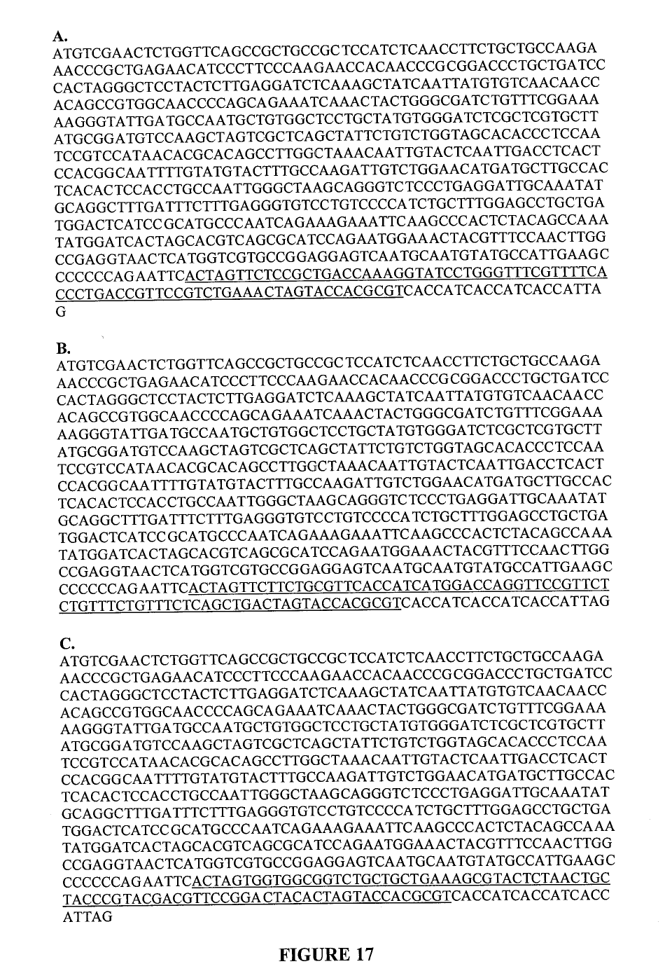

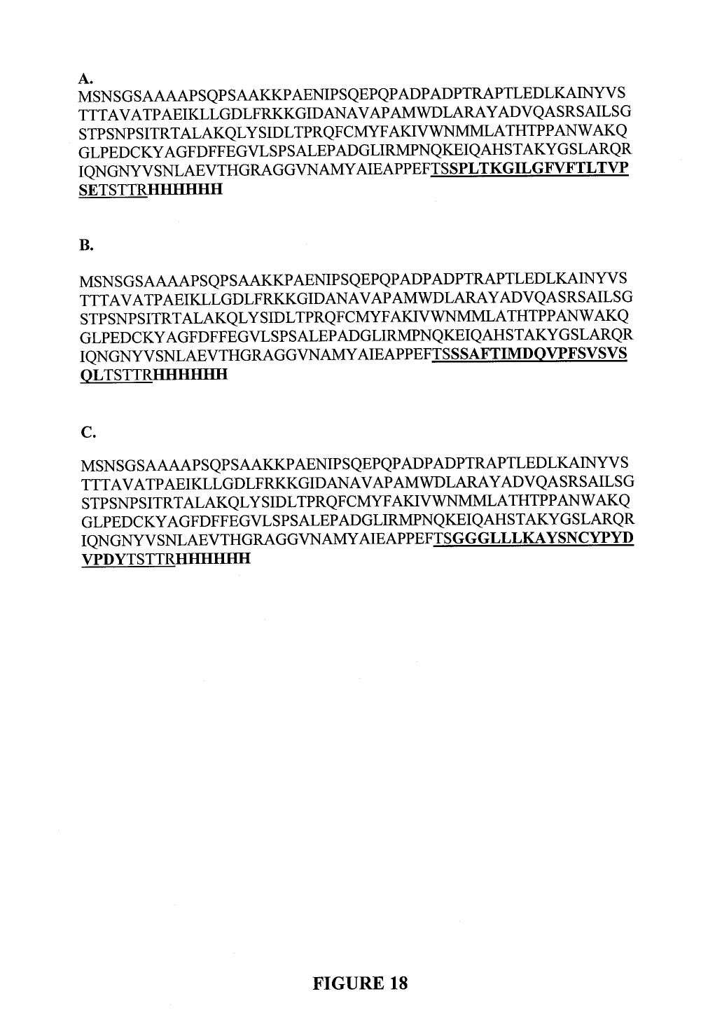

Using the MaMV-SM construct, two different fusions were generated. The first (MaMV-M1 or MaMV Flu-M1) included the CTL epitope derived from the M1 protein of influenza. The nucleotide sequence encoding the MaMV-M1 protein is shown in FIG. 17A (SEQ ID NO:66), and the amino acid sequence of MaMV-M1 is shown in FIG. 18A (SEQ ID NO:67). The second (MaMV-gp100) included the CTL epitope derived from the gp100 protein. The nucleotide sequence encoding the MaMV-M1 protein is shown in FIG. 17B (SEQ ID NO:68), and the amino acid sequence of MaMV-M1 is shown in FIG. 18B (SEQ ID NO:69).

A third fusion was also generated using a second MaMV CP (MaMV gl-SM) and the F3 peptide derived from the HA of an influenza H3 strain (KAYSNCYPYDVPDY (SEQ ID NO:72)). MaMV gl-SM has the sequence of the MaMV SM construct but includes a GGGLLL spacer. The nucleotide sequence encoding the MaMV gl-SM protein is shown in FIG. 10C (SEQ ID NO:64), and the amino acid sequence of MaMV gl-SM is shown in FIG. 11C (SEQ ID NO:65).

The fusion of MaMV gl-SM and the F3 peptide resulted in the construct MaMV gl-F3. The nucleotide sequence encoding the MaMV gl-F3 protein is shown in FIG. 17C (SEQ ID NO:70), and the amino acid sequence of MaMV gl-F3 is shown in FIG. 18C (SEQ ID NO:71).

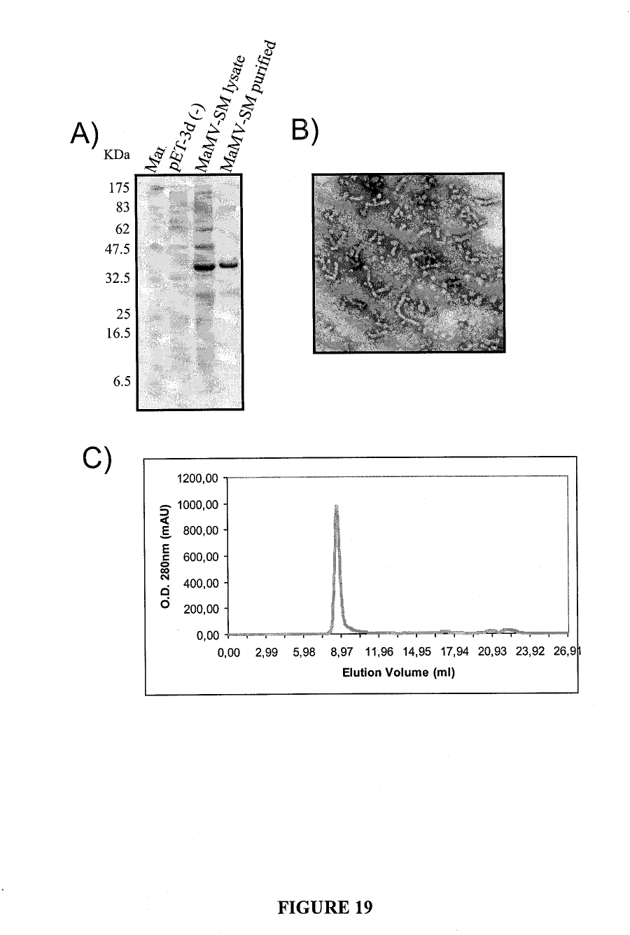

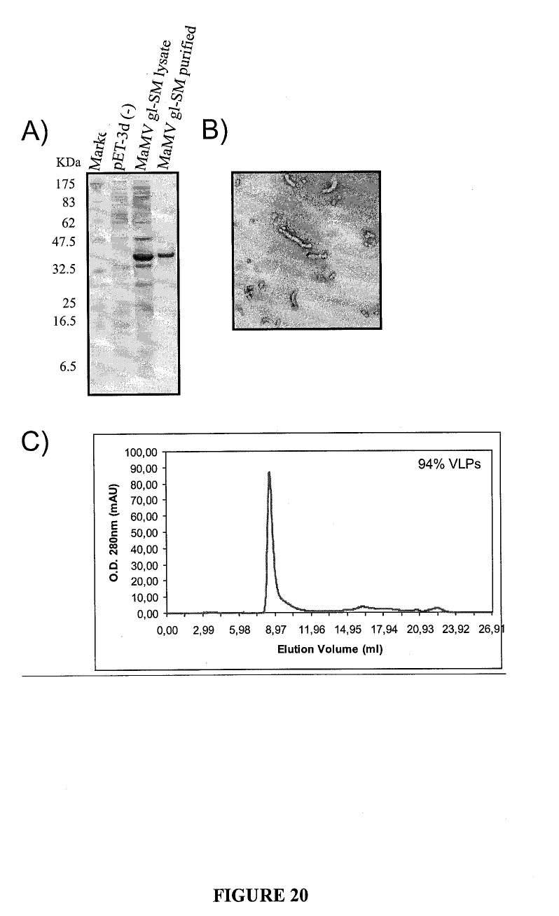

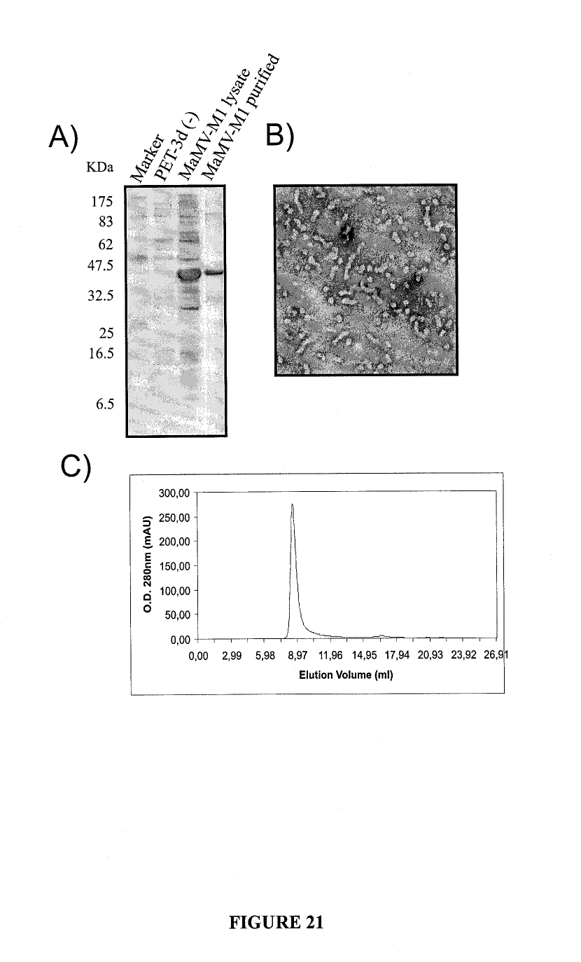

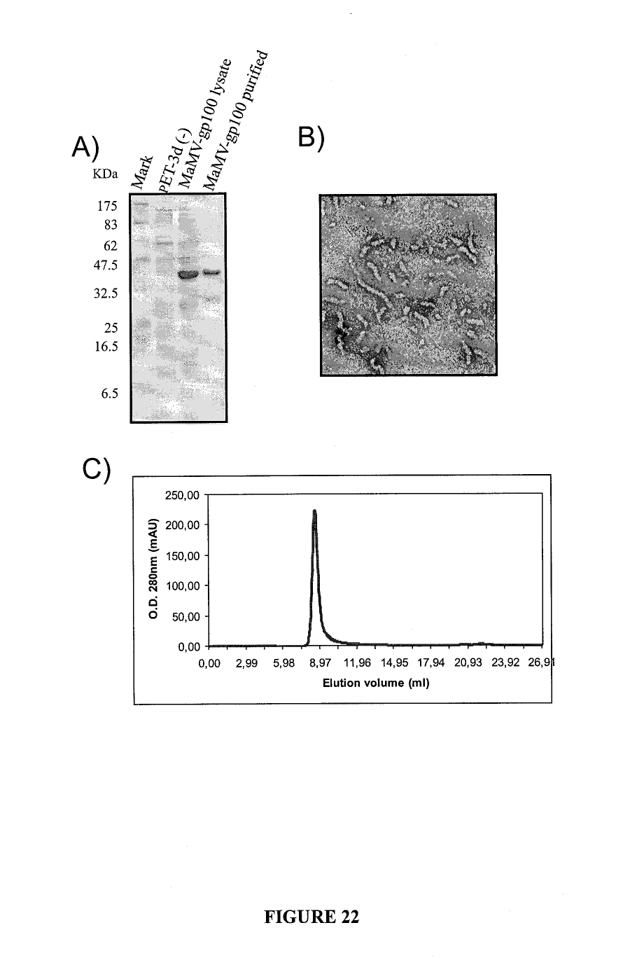

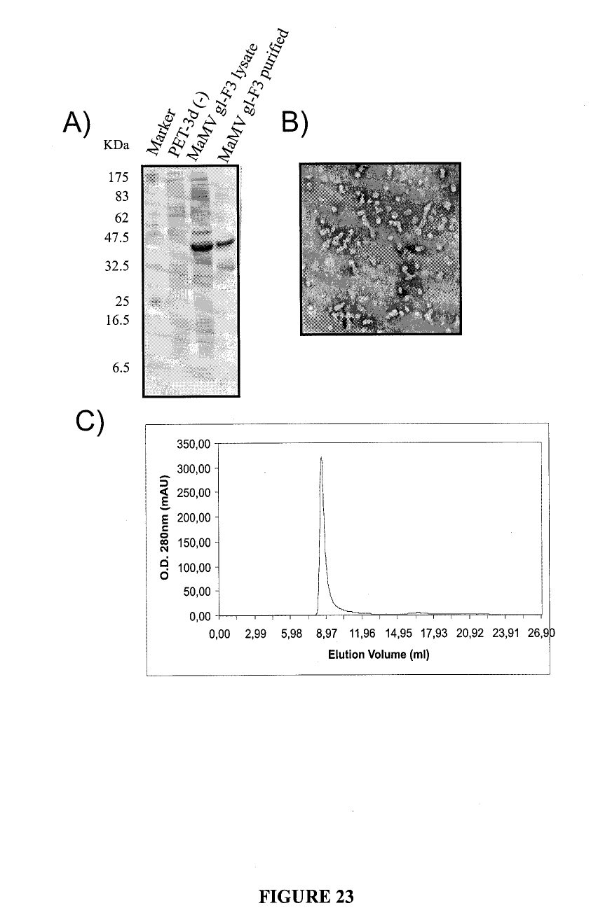

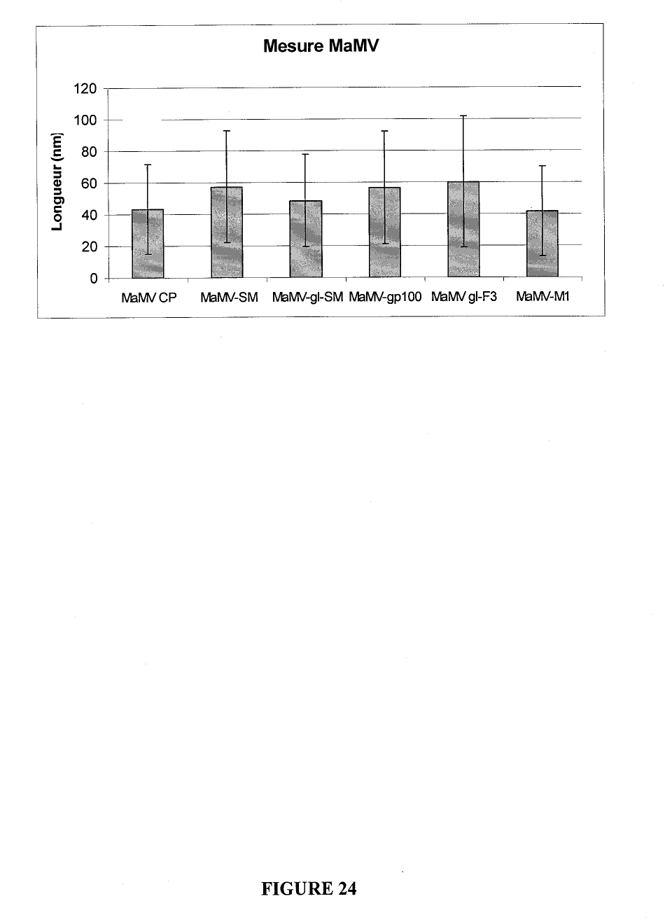

The purification profile of the respective coat proteins, electron microscopy photograph of the resulting VLPs and the FPLC profile of the VLPs are shown respectively in FIG. 19 (MaMV-SM), FIG. 20 (MaMV gl-SM), FIG. 21 (MaMV-M1), FIG. 22 (MaMV-gp100) and FIG. 23 (MaMV gl-F3). The length of the VLPs of each of these recombinant proteins was measured and no significant difference between them was observed (FIG. 24). The average length of the VLPs is 60 nm, but some VLPs exceeding 200 nm were measured.

The results described above demonstrate that the MaMV CP can tolerate the fusion of several different peptide sequences without losing its ability to form VLPs.

### Example 12

### Internalization of Malva Mosaic Virus VLPs by Lymphocytes

The ability of different sources of antigen-presenting cells (APCs) to take up and internalise MaMV VLPs was evaluated using previously described techniques (Leclerc D, et al. (2007) J Virol 81: 1319-1326) and compared to uptake and internalisation of VLPs derived from the coat protein of papaya mosaic virus (PapMV).

In brief, VLPs formed from PapMV CP, MaMV CP or MaMV-M1 (see Example 11) were conjugated with a fluorescent label (Allophycocyanin or APC), pulsed on CD40-activated B lymphocytes for 20 hours. CD40-activated B lymphocytes are efficient APCs, with similar properties compared to dendritic cells (DCs). Cells were then washed and fluorescence was evaluated by flow cytometry.

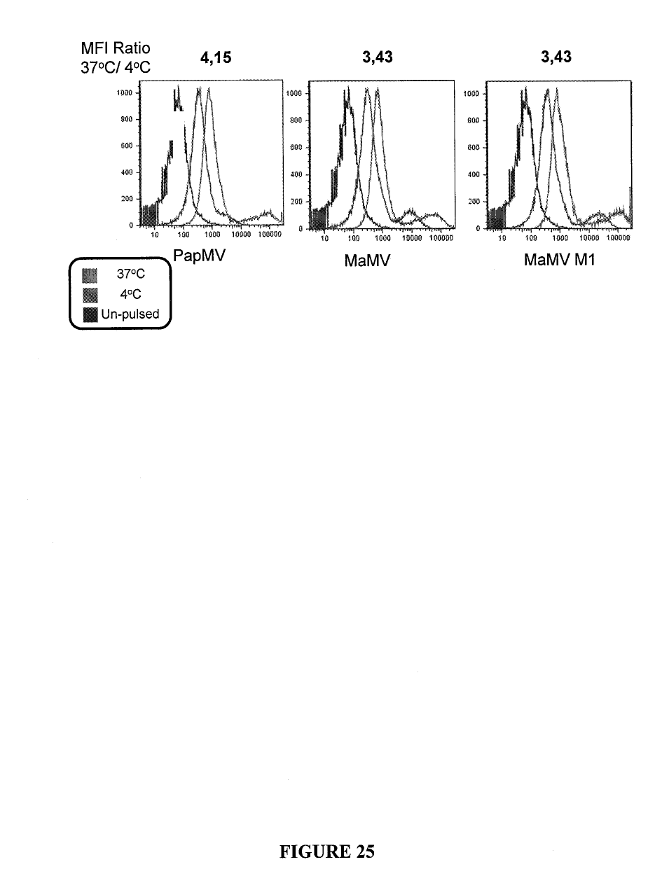

The results are shown in FIG. 25 and demonstrate that PapMV, MaMV and MaMV-M1 VLPs are internalized with similar efficiency. Staining at 4° C. also suggests that all VLPs can bind to the cell surface. The presence of 2 peaks (mid and high mean fluorescent intensity or MFI), suggests that the VLPs are differently internalized into/associated with the cells, however, there was no major difference between MaMV and MaMV-M1 VLPs.

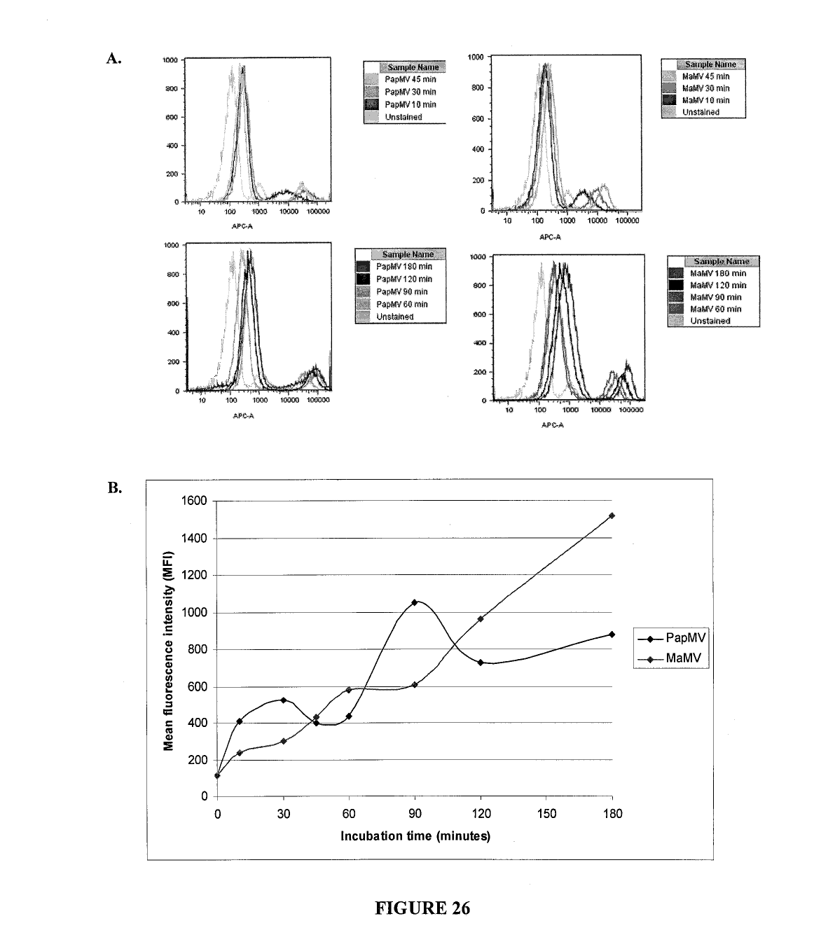

A hetero-hybridoma cell line of B and T lymphocytes was next exploited as a source of APCs. This cell line (T2 cells) is deficient in the transporter associated with antigen processing (TAP). A time course uptake assay with T2 cells was conducted in which the T2 cells were pulsed at 37° C. at the times indicated in FIG. 26, washed, and analyzed by flow cytometry.

The results are shown in FIGS. 26A & B and demonstrate that PapMV, MaMV and MaMV-M1 VLPs are internalized with similar efficiency in time. Again, 2 peaks (mid and high MFI) were observed, which suggests that the VLPs are also differently internalized into/associated with T2 cells. According to the increase of MFI over time, MaMV VLPs appear to be taken up more effectively. However, it is possible that the PapMV VLPs are degraded more rapidly, resulting in an apparent lower total MFI.

These results indicate that MaMV VLPs and PapMV VLPs are internalised into APCs with comparable efficacy and thus suggest that they will have comparable efficacy as a vaccine platform.

### Example 13

### Ability of Malva Mosaic Virus VLPs to Elicit MHC Class I Presentation of CTL Epitopes

PapMV has a demonstrated capacity to mediate MHC class I epitope cross-presentation under a proteasome-independent mechanism (Leclerc D, et al. (2007) J Virol 81: 1319-1326). As shown in Example 12, both PapMV and MaMV bind cells and are internalized similarly. As such, the ability of MaMV to elicit the presentation of covalently-linked MHC class I epitopes in a similar manner to PapMV was next investigated using previously described techniques (see Leclerc et al. (2007) supra).

In brief, VLPs derived from PapMV coat protein or MaMV coat protein with the HLA-A*0201 influenza M1 epitope inserted at the C-terminus (PapMV-M1 and MaMV-M1; see Example 11) and a synthetic peptide corresponding to the influenza M1 epitope, were pulsed on T2 cells for 20 hours. Cells were washed, and T lymphocytes specific to the M1 influenza epitope linked to the relevant HLA-A*0201 MHC class I molecule were added for an additional 20 hours. Supernatants were harvested and interferon (IFN)-γ secretion was evaluated by ELISA. IFN-γ is secreted by T cells that recognize the MHC/peptide complex.

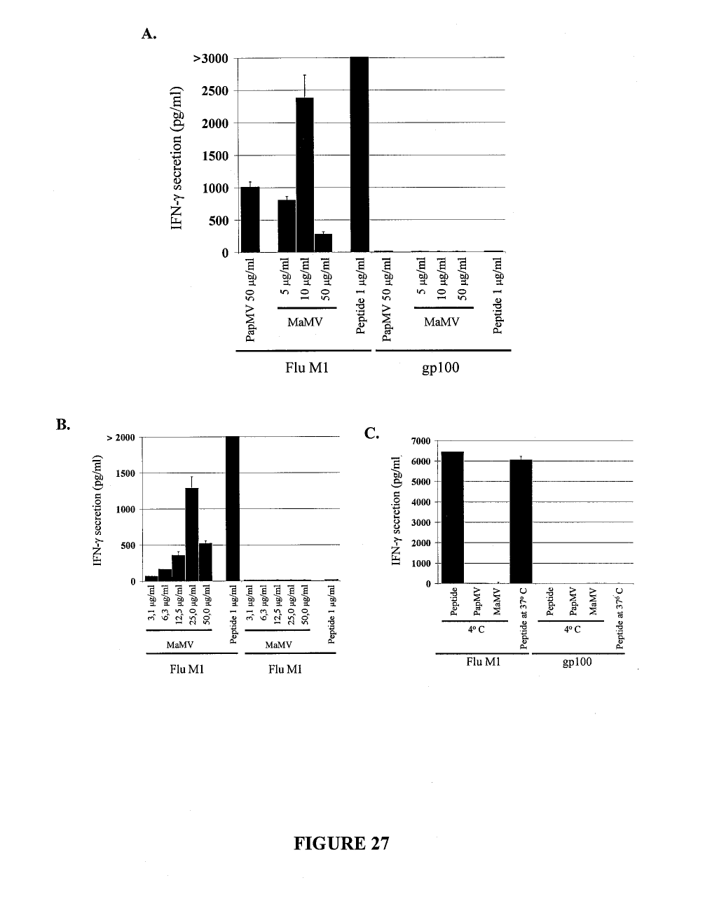

Data are presented in FIG. 27A. Gp100 serves as a negative specificity control and must be negative. The results show that both PapMV and MaMV VLPs have the capacity to cross-present the M1 epitope. For MaMV VLPs, improved recognition was observed at 10 μg/ml, with a marked decrease in recognition when pulsed at 50 μg/ml.

A similar experiment was conducted with MaMV-M1 to evaluate the dose response (FIG. 27B). Gp100 serve as a negative specificity control and must be negative.

As proteases are present in the human serum used to cultivate the APCs in the above experiments, it is possible that the pulsing of APCs is achieved after VLP degradation in the culture media, and not from VLP internalisation, processing and MHC loading after internalisation in APCs. To exclude this possibility, T2 cells were pulsed at 4° C. with the MaMV-M1 VLPs. The lower temperature being employed to inhibit any potential degradation of the VLPs. If peptides are released externally, they should load MHC class I from outside the cells, however, as shown in FIG. 27C this was not the case (the experiment shown in FIG. 27B was performed at the same time and served as positive control at 37° C.). As a control, synthetic peptides were similarly pulsed and were recognized when pulsed at either 4 or 37° C.

The results shown in FIGS. 27B & C demonstrate that a dose response correlates the recognition efficiency for MaMV-M1. However, pulsing at a higher dose (50 mg/ml) resulted in marked decrease recognition (FIG. 27B). The M1 epitopes were shown not to be released by degradation in the culture media containing human serum (FIG. 27C). Finally, since the above experiments and those in Example 12 were conducted in T2 cells, which are TAP-deficient, these results further confirm that the cross-presentation mechanism is not proteasome/TAP mediated.

### Example 14

### Sensitization of Resting Epitope-Specific T Lymphocytes by Malva Mosaic Virus VLPs

Using resting T lymphocytes from 4 different donors (#405, #542, #614 and #621), the capacity of VLP-pulsed APCs to elicit the expansion of influenza M1 protein-specific T cells was evaluated. This assay is referred to as “T cell sensitization” or an “in vitro vaccination.” In such an assay, it is possible to evaluate if VLPs carrying the M1 epitope are efficient in expanding specific T cells in a 3-week culture assay.

For each donor, autologous APCs (CD40-activated B lymphocytes) were first cultured. These cells were pulsed with either PapMV VLPs or MaMV VLPs carrying the M1 epitope (see Example 11) (25 μg/ml) or with a synthetic peptide comprising the M1 epitope. Pulsed APCs were then co-cultured with autologous blood T lymphocytes for 7 days, followed by a re-stimulation with the same pulsed-APCs. After a total of 21 days of culture, the specificity of the cultured T lymphocytes to the relevant influenza M1 epitope was evaluated.

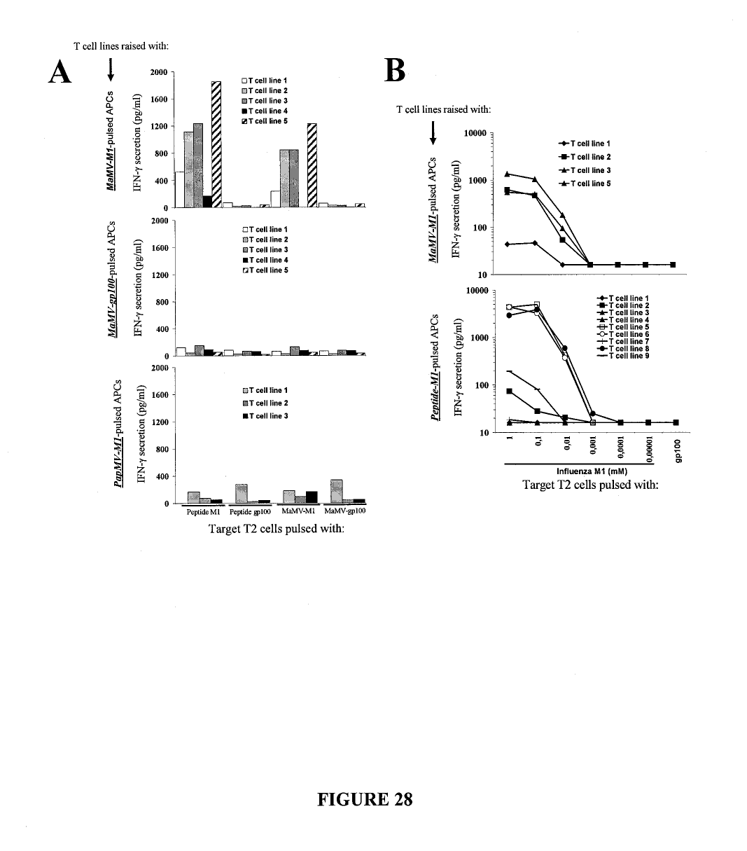

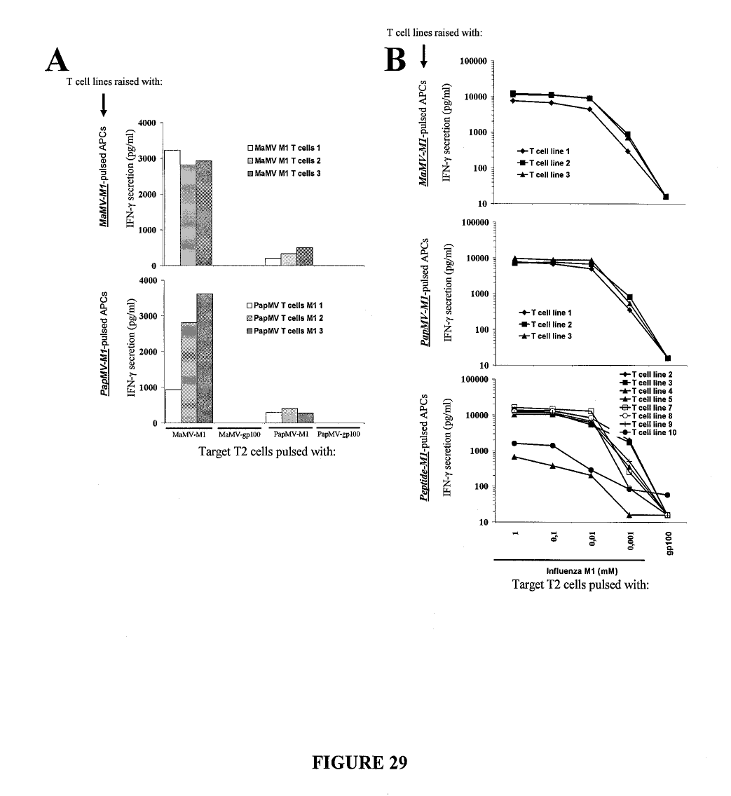

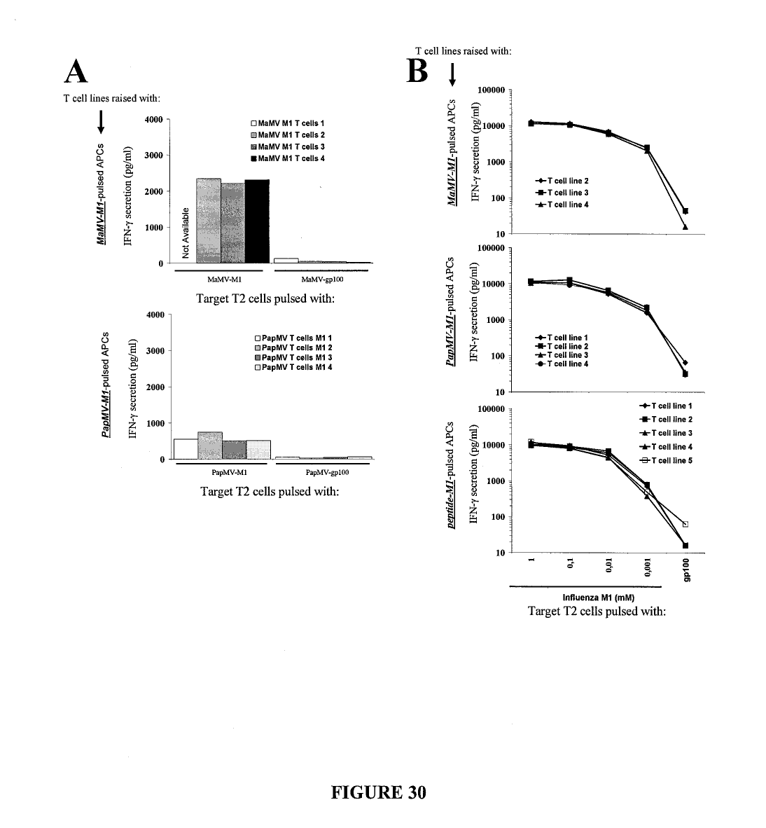

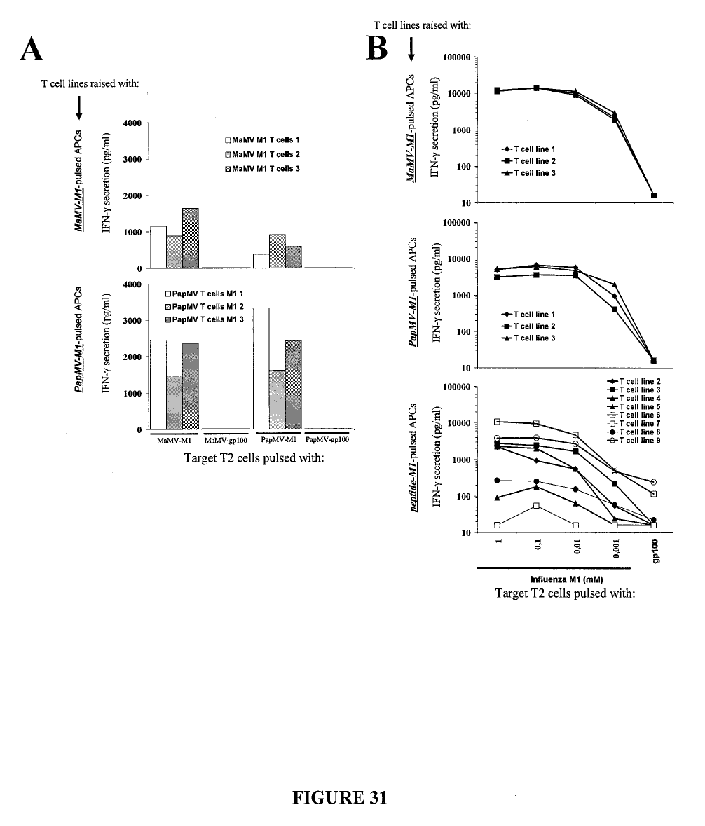

The specificity of raised T lymphocytes for each donor is presented in FIG. 28-31. For donor #405 (FIG. 28), MaMV-M1 VLPs resulted in the expansion of specific T-cells work, but PapMV-M1 VLPs did not. Some peptide specific lines were observed to be more avid when compared to MaMV-M1 VLPs. For donor #542 (FIG. 29), T cell lines raised with either PapMV-M1 VLPs or MaMV-M1 VLPs better recognize MaMV-M1. Overall, for this donor, VLPs tended to generate T cells with higher avidity compared to peptide pulsing. For donor #614 (FIG. 30), T cell lines raised with MaMV-M1 VLPs better recognize VLP-M1. Overall, T cells generated with VLPs raise T cells with higher avidity compared to peptide-pulsing at low peptide concentration (0.001 mM; note that this is a logarithmic scale). For donor #621 (FIG. 31), T cell lines raised with either PapMV-M1 VLPs or MaMV-M1 VLPs recognize VLP-M1. MaMV-M1 T cell lines appeared to have higher avidity compared to PapMV-M1 T cell lines, and VLP T cell lines were overall superior to peptide-pulsing.

In conclusion, both MaMV-M1 VLPs and PapMV-M1 VLPs were generally efficient in generating antigen-specific T lymphocytes in this in vitro T cell sensitization assay and, overall, both MaMV and PapMV VLPs performed better than peptide alone. Surprisingly, no anti-platform T lymphocytes were observed for either MaMV or PapMV VLPs. Overall, the MaMV and PapMV VLPs appeared to be equivalent. Variation within donors was expected.

Examples 12 to 13 clearly demonstrate that the MaMV-gp100 and the MaMV-M1 were capable of inducing cross-presentation of these CTL epitopes on MHC class 1 of human APCs and of inducing proliferation of CD8+ specific cells. These results strongly indicate that the MaMV platform is capable of triggering a CTL response in humans or animals.

In addition, Examples 12 to 14 demonstrate that the MaMV platform has immunogenic properties that are similar to those of the PapMV platform even though the sequences of the two coat proteins are divergent on 69% of their amino acids. Both platforms show enormous potential in vaccine development and it is expected that they could be used together in the same vaccination protocol to optimise the immune response to a given epitope or used in alternation in the same patient to avoid cross neutralisation by antibodies directed to the platform of the previous treatment.

### Example 15

### Ability of Malva Mosaic Virus VLPs Fused to Antigens to Elicit an Immune Response In Vivo

VLPs comprising MaMV-gp100, MaMV-M1 or MaMV gl-F3 (see Example 11) were tested to verify that an immune response toward the fused peptide could be detected in Balb/C mice.

In brief, mice received two subcutaneous injections at 14 day intervals as shown in Table 5. Blood was collected before each injection (at day 0 & 14) and also 14 days after the last injection (day 28).

TABLE 5
Mouse Immunizations

| | Amount of protein | |
|---|---|---|
| Protein injected | injected/innoculum | Number of mice/group |
| MaMV-SM | 100 μg | 10 |
| MaMV gl-F3 | 100 μg | 10 |
| MaMV gl-SM + F3 | total of 100 μg (= 94.3 μg | 10 |
| | of MaMV gl-SM + 5.7 μg | |
| | F3) | |
| MaMV-M1 | 100 μg | 10 |
| MaMV-gp100 | 100 μg | 10 |

ELISA was performed using blood drawn at days 14 and 28 to quantify the total anti-peptide and total anti-MaMV IgG titers.

The LPS content for each recombinant protein was evaluated and was in each case less than 15 EU/mg, which essentially makes the LPS content of the injected composition negligible.

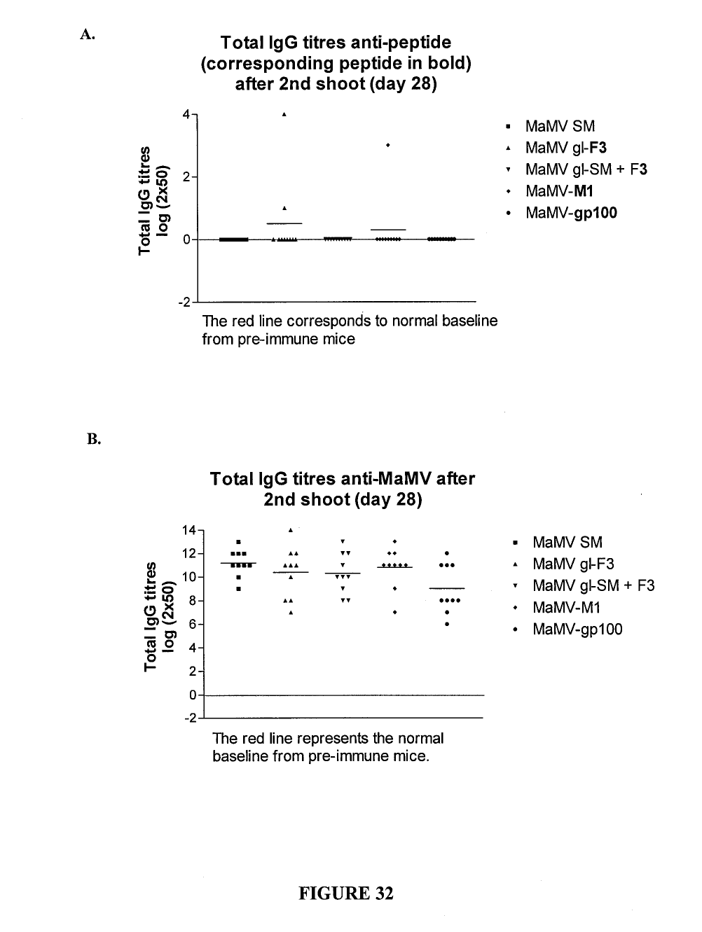

Surprisingly, most of the mice did not show an IgG response toward the peptide fused to the MaMV CP (FIG. 32A). Two of the ten mice treated with the MaMV gl-F3 construct showed seroconversion to the peptide, and one of the ten mice treated with the MaMV-M1 construct showed seroconversion to the peptide. No response was observed in mice treated with the MaMV-gp100 construct or with the F3 peptide when the F3 peptide was not fused to the MaMV platform.

In this regard, it is important to note that the choice of the CTL epitopes derived from M1 or gp-100 protein was probably not optimal for assessing the humoral response in mice as it is very likely that the immune repertoire of the mice used for the experiment does not include B-cells that can recognise these peptides. This is likely also the case for the F3 peptide.

The previous Examples demonstrate that MaMV is a strong adjuvant. This Example confirms, however, that it is not possible to trigger a strong IgG response if the immune repertoire of the test animals cannot recognise the antigen. An adjuvant can only amplify a B-cell response if the B-cells specific to the antigen in question are available. This conclusion is supported by the fact that a very strong IgG response could be measured in all cases toward the MaMV CP showing clearly that the platform is highly immunogenic (FIG. 32B). This is an important characteristic for a vaccine platform as, in general, it is well recognised that for vaccine platforms such as HBV VLPs or PapMV VLPs that the intrinsic immunogenicity of the platform itself drives the immune response directed to the fused epitope or peptide at the surface of the VLPs. Therefore, since the MaMV VLPs were shown to be highly immunogenic, use of a more appropriate epitope (for example, the M2e epitope of the M2 protein of influenza (see Denis et al., 2008, Vaccine, 26:3395-3403), is anticipated to produce a very strong immune response toward the peptide.

In addition, for future experiments, a spacer will be cloned between the fused peptide and the MaMV CP to push the peptide to the surface of the VLPs as this is expected to improve the cross link with B cells, which was not optimised in this experiment.

### Example 16

### Cross-Reactivity of Antibodies to Papaya Mosaic Virus VLPs and Malva Mosaic Virus VLPs

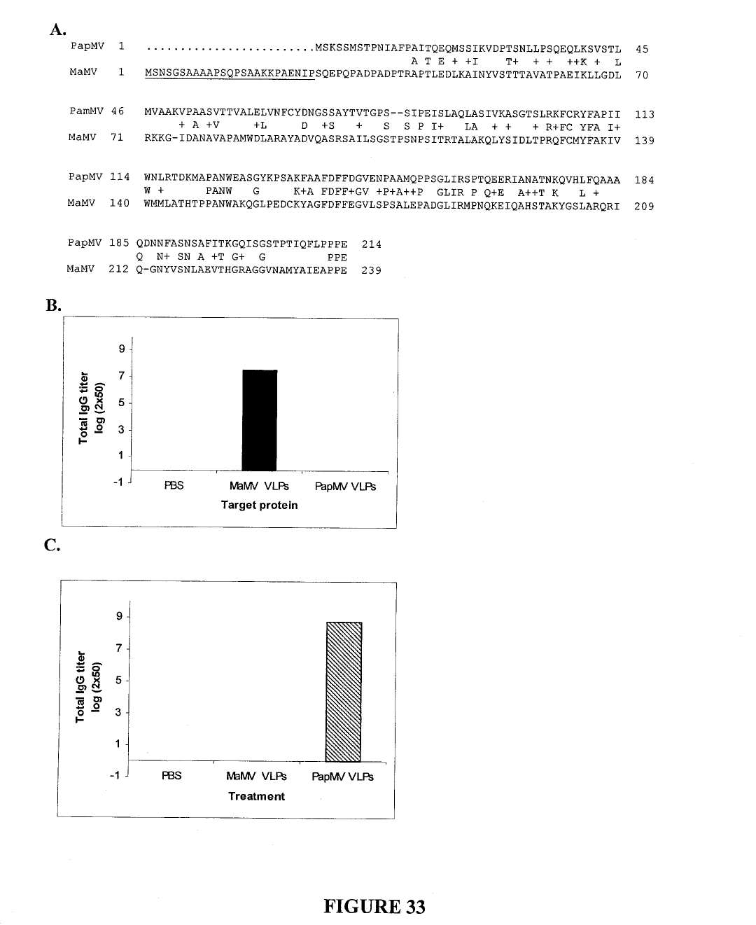

PapMV and MaMV CPs share only 31% sequence identity (see FIG. 33A). The difference in sequence is also apparent in the lack of cross-reactivity in antibodies specific for each coat protein. For example, FIG. 33B shows the results of an ELISA using antibodies directed to MaMV CP, which can be seen not to recognise PapMV VLPs. A similar experiment with antibodies directed to the PapMV CP also revealed that PapMV antibodies do not bind the MaMV platform (FIG. 33C). This result suggests that the surfaces of the two VLPs are very different. The two platforms could therefore be used together in a vaccine regimen to ensure a better and more efficient vaccination program since the antibodies directed to the first platform cannot interfere with the second one.

### Example 17

### Immunogenic Effect of Malva Mosaic Virus

The following protocol is an alternate protocol that can be followed in order to assess the immunogenic effect of compositions comprising MaMV and a weak immunogen.

The translation of innate immune response into antibody response is observed when adjuvants are co-administered to poor immunogenic vaccines. Adjuvants are substances capable of strengthen or augment the antibody or cellular immune response against an antigen. To determine whether MaMV is an adjuvant that can promote a long-lasting antibody response to other antigens, BALB/c mice can be immunized with the weak immunogens, ovalbumin (OVA) or hen egg lysozyme (HEL) either alone or together with MaMV. Other adjuvants such as CFA or LPS in combination with OVA or HEL can be used as positive controls. The IgG antibody titer specific for OVA or HEL can then be measured by ELISA at appropriate time points.

### MaMV Purification

MaMV for use in the following experiment can be purified as described in Example 1.

### Antigens and Adjuvants

LPS-free OVA Grade VI can be purchased from Sigma-Aldrich Chemical Co, St Louis, Mo. and hen egg white lysozyme (HEL) can be purchased from Research Organics Inc. Cleveland, Ohio. LPS from E. coli O111:B4 can be purchased from Sigma-Aldrich, St Louis, Mo.

### Immunizations

BALB/c mice, 6-8 weeks old, are bred and kept under conditions in conformity with good laboratory practice guidelines. To study the effects of the test adjuvants, groups of mice are immunized i.p. on day 0 with 2 mg of OVA or HEL alone or in combination with 30 mg of MaMV, CFA 1:1 (v/v), or 5 mg of LPS from E. coli O111:B4 (Sigma-Aldrich). Control mice are injected with saline solution only. Blood samples are collected from the retro-orbital sinus at various times. Individual serum samples are stored at −20° C. until analysis. Three mice are used in each experiment.

### Determination of Antibody Titers by ELISA

High-binding 96-well polystyrene plates (Corning®, New York, N.Y.) are coated with 1 mg/mL of MaMV, 100 mg/mL of HEL, or 150 mg/mL OVA in 0.1 M carbonate-bicarbonate buffer (pH 9.5). Plates are incubated for 1 h at 37° C. and then overnight at 4° C. Before use the next morning, plates are washed three times in PBS (pH 7.2) containing 0.05% Tween-20 (PBS-T) (Sigma-Aldrich). Nonspecific binding is blocked with 5% nonfat dry milk diluted in PBS (PBS-M) for 1 h at 37° C. After washing, mice serum is diluted 1:40 in PBS-M, and 2-fold serial dilutions are added to the wells. Plates are incubated for 1 h at 37° C. and then washed four times with PBS-T. Peroxidase-conjugated rabbit anti-mouse IgM (optimal dilution 1:1000) IgG, IgG1, IgG2a, IgG2b antibodies (Zymed, San Francisco, Calif.) or IgG3 (optimal dilution 1:3000) (Rockland, Gilbertsville, Pa.) is added, and the plates are incubated for 1 h at 37° C. and washed three times with PBS-T. Orthophenylenediamine (0.5 mg/mL; Sigma-Aldrich) in 0.1 M citrate buffer (pH 5.6) containing 30% hydrogen peroxide is used as the enzyme substrate. The reaction is stopped with 2.5 N H₂SO₄, and the absorbance is determined at 490 nm using an automatic ELISA plate reader (Dynex Technologies MRII, Chantilly, Va., USA) with BIOLINX 2.22 software. Antibody titers are given as −log 2 dilution×40. A positive titer can be defined as 3 SD above the mean value of the negative control.

### Example 18

### Induction of Resistance to Sublethal Doses of Influenza Virus by Malva Mosaic Virus and Malva Mosaic Virus VLPs

The ability of MaMV or recombinant MaMV VLPs to induce resistance to sublethal dose of influenza virus in the absence of antigen can be tested in mice. In brief, mice will be treated by the intranasal route with 100 ug of MaMV, 3 times at 7 day intervals. 2 days after the last treatment, the animals will be submitted to sublethal dose of Influenza virus (A/WSN/33) (1 LD₅₀). The amount of virus replicating in the animal will be measured by sampling the secretion in the nose at 2 day intervals after the infection. The temperature and weight of the animals will be monitored daily for 7 days after challenge. The morbidity due to infection will be scored 10 days after infection.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An immunogenic composition comprising Malva mosaic virus (MaMV) or a virus-like particle (VLP) comprising MaMV coat protein and a pharmaceutically acceptable carrier.

2. The immunogenic composition according to claim 1 further comprising one or more antigens.

3. The immunogenic composition according to claim 2, wherein said one or more antigens are attached to said MaMV or VLP.

4. The immunogenic composition according to claim 2, wherein said immunogenic composition comprises a VLP and said one or more antigens are genetically fused to the coat protein comprised by said VLP.

5. The immunogenic composition according to claim 1, wherein said MaMV coat protein comprises an amino acid sequence at least 80% identical to the sequence as set forth in SEQ ID NO:2.

6. The immunogenic composition according to claim 1, wherein said MaMV coat protein comprises an amino acid sequence at least 90% identical to the sequence as set forth in SEQ ID NO:2.

7. The immunogenic composition according to claim 1, wherein said MaMV coat protein comprises an amino acid sequence as set forth in SEQ ID NO:2.

8. A method of potentiating an immune response to one or more antigens in an animal comprising administering to said animal an effective amount of the immunogenic composition according to claim 1.

9. The method according to claim 8, wherein said immune response comprises the production of antibodies.

10. The method according to claim 8, wherein said immune response comprises a cellular response.

11. The method according to claim 8, wherein said immunogenic composition is administered by injection.

12. The method according to claim 8, wherein said immunogenic composition is administered intranasally.

13. The method according to claim 8, wherein said animal is a mammal, a bird, a fish or a reptile.

14. The method according to claim 8, wherein said animal is a human.

15. A method of vaccinating an animal against a disease, disorder or infection comprising administering to said animal an effective amount of immunogenic composition comprising Malva mosaic virus (MaMV) or a virus-like particle (VLP) comprising MaMV coat protein and one or more antigens.

16. The method according to claim 15, wherein said MaMV coat protein comprises an amino acid sequence at least 80% identical to the sequence as set forth in SEQ ID NO:2.

17. The method according to claim 15, wherein said MaMV coat protein comprises an amino acid sequence at least 90% identical to the sequence as set forth in SEQ ID NO:2.

18. The method according to claim 15, wherein said MaMV coat protein comprises an amino acid sequence as set forth in SEQ ID NO:2.

19. The method according to claim 15, wherein said one or more antigens are attached to said MaMV or VLP.

20. The method according to claim 15, wherein said immunogenic composition comprises a VLP and said one or more antigens are genetically fused to the coat protein comprised by said VLP.

21. The method according to claim 15, wherein said immunogenic composition and said one or more antigens are administered by injection.

22. The method according to claim 15, wherein said animal is a mammal.

23. The method according to claim 15, wherein said animal is a human.

24.-29. (canceled)

30. A virus-like particle (VLP) comprising Mavia mosaic virus (MaMV) coat protein.

31. The VLP according to claim 30, wherein said MaMV coat protein comprises an amino acid sequence at least 80% identical to the sequence as set forth in SEQ ID NO:2.

32. The VLP according to claim 30, wherein said MaMV coat protein comprises an amino acid sequence at least 90% identical to the sequence as set forth in SEQ ID NO:2.

33. The VLP according to claim 30, wherein said MaMV coat protein comprises an amino acid sequence as set forth in SEQ ID NO:2.

34. The VLP according to claim 30, further comprising one or more antigens attached to said VLP.

35. The VLP according to claim 30, further comprising one or more antigens genetically fused to the MaMV coat protein.

36. A method of preparing a virus-like particle (VLP), said method comprising the step of expressing a Malva mosaic virus (MaMV) coat protein in a host cell.

37. The method according to claim 36, wherein said MaMV coat protein comprises an amino acid sequence at least 80% identical to the sequence as set forth in SEQ ID NO:2.

38. The method according to claim 36, wherein said MaMV coat protein comprises an amino acid sequence at least 90% identical to the sequence as set forth in SEQ ID NO:2.

39. The method according to claim 36, wherein said MaMV coat protein comprises an amino acid sequence as set forth in SEQ ID NO:2.

40. The method according to claim 36, wherein said MaMY coat protein is a fusion protein comprising one or more antigens genetically fused to the coat protein.

41. The method according to claim 36, wherein said host cell is a bacterial cell.

42. The method according to claim 36, wherein said host cell is an E. coli cell.

43. A fusion protein comprising a Malva mosaic virus (MaMV) coat protein fused to an antigen.

44. The fusion protein according to claim 43, wherein said MaMV coat protein comprises an amino acid sequence at least 80% identical to the sequence as set forth in SEQ ID NO:2.

45. The fusion protein according to claim 43, wherein said MaMV coat protein comprises an amino acid sequence at least 90% identical to the sequence as set forth in SEQ ID NO:2.

46. The fusion protein according to claim 43, wherein said MaMV coat protein comprises an amino acid sequence as set forth in SEQ ID NO:2.

47. An isolated polynucleotide encoding the fusion protein according to claim 43.

48. A host cell genetically engineered with the polynucleotide according to claim 47.

49. (canceled)

50. The method according to claim 8, wherein said one or more antigens are attached to said MaMV or VLP.

51. The method according to claim 8, wherein said immunogenic composition comprises a VLP and said one or more antigens are genetically fused to the coat protein comprised by said VLP.

52. The method according to claim 8, wherein said MaMV coat protein comprises an amino acid sequence at least 80% identical to the sequence as set forth in SEQ ID NO:2.

53. The method according to claim 8, wherein said MaMV coat protein comprises an amino acid sequence at least 90% identical to the sequence as set forth in SEQ ID NO:2.

54. The method according to claim 8, wherein said MaMV coat protein comprises an amino acid sequence as set forth in SEQ ID NO:2.


SEQUENCE LISTING (78 sequences)

SEQ ID NO: 1 - DNA, 6858 bp, Malva mosaic virus
gaaaacaaaacaacacaaacacactctactcagtagtaaccatataaggacaataaccgtttaacaacactatcgtcccatatggcaaaagttagagcgtcgttggaaaggattagagacccatcagtccaaactgcactctcggaggcagcttacactcatgtccgtcctgtcctaaaagaagccttggtcaattgcccttatgcactgaccgacgcggaagcagattgtttagaaaattttggtattacaataaacccatacgccactcaaacccacactcacgcagcttgtaaagcaatagagaatagaatgttagagatagtaggcaagcacttgcccaaagagaaagttaccatgctcttcctaaagaaaagtaaattaaggtatctgcgcagagccgctgccctaaatgacgttttcattaacaaagacatggaaccaaaagacctctttaggtatgatagagatacaataagaaatagtcttcaaaatgtagacactagtttagcttacatgtctgacacactacatttcatgaactttaactcagtagctcagctctttgtggattctccaaaattgaccactttattggcaacagtagtgttaccagtagaagcgctacatcgcagatcgtctctatatccagcgttatattcaatcaactaccacccacaagggtttgaatatattcctgggaaccatggaggaggggcttactttcacccatatgacaccttagattggctgaaagtccgcaagatccttgcaaaagacttttatcatcttgatgagaaatttactttaacattccagatggtagaaagcatcggggccaatcacctcttcatcattcacaaagccgacatgataactcccggcatgagaacattctgcagagactctctggtgactttaccaaaaatcttttgccctgattcgatgaacgctaacagacctttatcgaaaaccaaagctatgcaattgatgttgtattgcaaatctgttaagcaagttacggagagagacatctatgctaaattccgacaaattattcctaccatagagttggaactgtatgaaccagacgagataattcacctttgcaactatctcaacttcatgactcaactcagttctataacctcctacgaagatttgttgggtaacagtttatggcgtagactcactcagccaatcaaggctcaggttaaaaaatttttggacttcttccgcggtcaagatgatttcaccaaattgctcggcgtactaaaatgggaagcttttagctactccctgtgtccctccgattttcacgccactttcatcagcaaatctgctcaagattttgcgacatcacaagaagtccccattgacgaagctgttgagacaatacgacattgtcgcaataatcccaactttgtcagtctggatgattttgaagtgtcgagtctaccagattttttatctcccccagcccaaatggaggacttttctacagatgaaagtgtggatgacacaccgacgtgcccgacaaccaaccttcctaaccaggacgttccggaagaacatcaagtggtgatccacaacagcgattggcaggtagacacaatattgcctatcatacgcaacctaccatacacacaactgaagggtcgaaaatgttactttttgaccacctcacccgacatagattatgggcacgactctgttagctaccaagcttgtccttgggatgaagttcttagtgacatcttgctaactacaggacaaagagcaaatgcatgcctcatccaggtttacgagcaagatgccaaaataccattacacagagatgatgagaaagtttacgcagggcatcccattctaactatgaactttggccaggcatctttctcgttttccaatggagaaacttttgaactaacagacggtcagtggttcgtcatgagtggcaagtacttgacacagtataaacatgcagcttttgctaagtctgtcggaaggatatccctgaccttccgaacacatgttaagacactggcaggccaccccatttcgagtcgtagccttgaacctacgctgaatgacgtagttgaaccggaagaggctactaagaacgatgtctcagatgctcctgaagtgccaaaatcttttagaaatccactgagttcacaagcatcggagccaatcccgtcaaccactgactcacttccctggaatgcttggaagcctttactcaaggaacatggcttcgaaggcgatcaacagcagctggactcatctgggttcatgattttgcccattacggacatcaagaaagtgccacatgtaccctaccccagtgaagtgccagaagctttacagaaagctttgatagccatcaaacgctaccccgtggctataactctagaccacaagagagcgggttcttatgcttctgacatcaagaatagccggacagggaaactgttagcacaaaaagatgataaatggaaagcagcatttgcttacaagatgcagcacgaagaccatgtggtggtcggtacagtcatacacggttgtggaggatcagggaaatctcataccctacaaaactggatgaggactttgaaggcagaccagagtgatgtaactgtagtcacaccaaccattttgttacgaaatgattggagcactaagttacccatcttgccagcagaaaccttcaaaacttttgagaaagccattgtgcagccatgtaacccagtagtcatcttcgatgattacacgaagcttccgcctggcatcattgaagcacttgtgatgcaccatcgtaatgtgagcttcattattctaactggagacagtaggcagtctgtgtaccatgaaatgaatcctgaggcttacatctccgctttaccagaagctgttgaagtttttgagccttattgcgaattctacgtcaatgcctcacacaggaatgtaaaacagctggctaataaattaggcgtatattcggagagagaaggcaaattgaaagtcaatttcgcctcccatcatttgaaagcttccagaacccctattttggtgccatctactatgaaaaggaacgccatggctgacatgggccaccacagcatgacctatgcaggctgccaaggcctgactgccccaaaagtgcaaatcctcttagacaaccacacccaattttgttccgacagggtgttgtatacctgcctttcccgagctgtggacacaattcatttcattaacacaggtcccaccactggggattattggactaagctggaaagtactccatacctgaaagctttcatcgacacgtacagggacgagaaaacggaattgtacaacagctcacctgccgatgatagcccaactgaaccagaggctccgaaaactcacttcccagttgccccaaaaccacttttggaacctctggtggctgaactagctgataaagagagtagagaaattttttctcgcagcacagggtattcaaacaccattcaaactaacgaccccgtggtgcagcttttccaacatcaacaggcgaaagacgagacattgtattgggcaactattgaagcccgactctctatctctacccccgaggagaaccttagagagttcaaaatgaaagctgatgttggggatattctcttcatgaattatgccaagctaatgcaactaccagcagaacccgtaccattcgaagaaaggctttgggagatctcagcggcagaggtgcgcaatacctatctgagtaagccaataggaaatttggtgaatgcagctactaggcagagcccagatttccctgcgaacaaaattgcgctcttcctcaaatcccagtgggtcaagaaggttgaaaagctgggtgctataaaagtcaaacctggccagaccattgcctcctttcatgcaagaaacggtgatgttgtacggtacgatgcaagatacatacgcaagatgagggcccggtttcagcccgataatatctttattaactgtgaagctacgcccgaagatctaaacaaatttgttaagaccaagtggcacttcaacagacctgcccacacaaatgatttcacagcgtttgaccaatctcaagatggcgctatgctacaatttgaagttatgaaagccaagttcttcaatataccagcagacattattgaaggatacatatacataaagctaaacgctgtgattttccttggcactttgggcatcatgaggctttcaggggagggacccactttcgatgccaacactgaatgttccattgcatataatgccaccagatttcatattgaagatgacacagcacaagtgtatgcaggagacgatatggccttagatagaatcagcacagaaaagagcagttttgcaaagctggcacatcaactcaaattgacgtccaaacctcaatttccaagacaggtccgtggggattatgcagagttttgtggttgggtgttgactccagctgggattttgaagcattctcttaaaatgcaggcctcgatccaactccaaaagaagatcaacaacatagcacagagtgctaggtcttatgctttggatctgcgatatgcctatcaaatggggatgaattacaggagcacctcactgaagaagaagcagatttccaccagcagtctatcagagacatgcatctgttgcaccaggaggacgtgttggttaacggcagtgctagcccacccagagacactgagttgcctaagctatgcggttccacggcaacacaaaaacgcaacaatttgaagaagaagaaaaagagtagggtggtggacatacttcctccccctactagccgggttaagttacccagtgttcgaaatggagttagattatctaattaaattgttagaatttaataattttccacgcacaaacctagatttctcgttacccttagtagtacacggcgttgctggctgtggcaagtcaacaataattagcaaattagctaaagctttccccactctagtagtcgcttcctttaccccacaaatcttagacggtaatacaggacggaaacaagtcgctgtggacggcagcccggttgatattttagacgagtatctaagtggtcccaccccttcagtgagattagcgcttttctgcgacccacttcaatattcttgcgaaaagcctcggttgcctcacttcatctcgctcacaactcatcggttctgcccactcacagcggacttcctgaattcaaaatttgggtgtgaaatcatttcactccgccaagacagttgtgaaatcgttgaagcagaccccttcgccactgatcctgaaggtgtcgttattacttttgaacccgaggttaaaagcatccttgaaaggcaccagtgttttccaactgatattagcacactttggggcaagaacttagaaaccgttagcgtgtacctcagctcatttgacacttgtttggattcttttagaacagatctgttcctaagcctcacacgacacactaagaagcttcttgtttttgacttcaatgcctggcctgacagctccgacgaactatgagcaggtttacaagattttagctattggtgtgctcagttgcttatctatctacgcacttagagcaagtcatctgccccacgttggtgacaacctccatcatttaccacacggcggaaattacgttgacggcactaagagaatctcctactttaaacctcacacctcacatggtgctaactacaagtggagtgccgcctgcgccatcgggtttctttcacttcttatcttcgcacaaactcgatttaattctcgtcctgtcagtacttctgttcgtgtatgcgctcattgcacctccaactccagtgtgccaagtgataatcaccggtgaatctgtcaaagttctaaactgtcctgcacccgataagattatagctagtattcaactcgccccttggaacggggttaagtttcctattttataggtttgaaaataaattaagtatcgttatatattgttgatattgaataaccactccatgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattctgaacgttagcaaacttaaattaacgtgtggtttatatagttttatttccacttatgctaataaagtataatt

SEQ ID NO: 2 - protein, 243 aa, Malva mosaic virus
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEF

SEQ ID NO: 3 - DNA, 732 bp, Malva mosaic virus
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattctga

SEQ ID NO: 4 - protein, 23 aa, Artificial Sequence
  Synthetic construct
SLLTEVETPIRNEWGCRCNDSSD

SEQ ID NO: 5 - protein, 23 aa, Artificial Sequence
  Synthetic construct
SLLTEVETPIRNEWGCRCNGSSD

SEQ ID NO: 6 - protein, 23 aa, Artificial Sequence
  Synthetic construct
SLLTEVETPTKNEWDCRCNDSSD

SEQ ID NO: 7 - protein, 23 aa, Artificial Sequence
  Synthetic construct
SLLTEVETPTRNGWECKCSDSSD

SEQ ID NO: 8 - protein, 23 aa, Artificial Sequence
  Synthetic construct
SLLTEVETPTRNEWECRCSDSSD

SEQ ID NO: 9 - protein, 8 aa, Artificial Sequence
  Synthetic construct
EVETPIRN

SEQ ID NO: 10 - protein, 8 aa, Artificial Sequence
  Synthetic construct
EVETLTRN

SEQ ID NO: 11 - protein, 8 aa, Artificial Sequence
  Synthetic construct
EVETPIRS

SEQ ID NO: 12 - protein, 8 aa, Artificial Sequence
  Synthetic construct
EVETPTRN

SEQ ID NO: 13 - protein, 8 aa, Artificial Sequence
  Synthetic construct
EVETPTKN

SEQ ID NO: 14 - protein, 8 aa, Artificial Sequence
  Synthetic construct
EVDTLTRN

SEQ ID NO: 15 - protein, 8 aa, Artificial Sequence
  Synthetic construct
EVETPIRK

SEQ ID NO: 16 - protein, 8 aa, Artificial Sequence
  Synthetic construct
EVETLTKN

SEQ ID NO: 17 - DNA, 22 bp, Artificial Sequence
  Synthetic construct
acatgtaagctaaactagtgtc

SEQ ID NO: 18 - DNA, 50 bp, Artificial Sequence
  Synthetic construct
gtgataaagttatgaccataacctatgtcgtaggatatgcattaactaat

SEQ ID NO: 19 - DNA, 50 bp, Artificial Sequence
  Synthetic construct
attagttaatgcatatcctacgacataggttatggtcataactttatcac

SEQ ID NO: 20 - protein, 12 aa, Artificial Sequence
  Synthetic construct
KYAGFDFFDGVT

SEQ ID NO: 21 - DNA, 27 bp, Artificial Sequence
  primer
ggtacatgtcgaactctggttcagccg

SEQ ID NO: 22 - DNA, 53 bp, Artificial Sequence
  primer
tacggatcctcaatggtgatggtgatggtggaattctgggggggcttcaatgg

SEQ ID NO: 23 - DNA, 750 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattccagcatcagcatcagcattga

SEQ ID NO: 24 - protein, 249 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPVMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFHHHHHH

SEQ ID NO: 25 - protein, 18 aa, Artificial Sequence
  Synthetic construct
AINYVSTTTAVATPAEIK

SEQ ID NO: 26 - protein, 25 aa, Artificial Sequence
  Synthetic construct
AINYVSTTTAVATPAEIKLLGDLFR

SEQ ID NO: 27 - protein, 8 aa, Artificial Sequence
  Synthetic construct
APTLEDLK

SEQ ID NO: 28 - protein, 26 aa, Artificial Sequence
  Synthetic construct
APTLEDLKAINYVSTTTAVATPAEIK

SEQ ID NO: 29 - protein, 9 aa, Artificial Sequence
  Synthetic construct
AYADVQASR

SEQ ID NO: 30 - protein, 25 aa, Artificial Sequence
  Synthetic construct
AYADVQASRSAILSGSTPSNPSITR

SEQ ID NO: 31 - protein, 9 aa, Artificial Sequence
  Synthetic construct
EIQAHSTAK

SEQ ID NO: 32 - protein, 16 aa, Artificial Sequence
  Synthetic construct
GIDANAVAPAMWDLAR

SEQ ID NO: 33 - protein, 41 aa, Artificial Sequence
  Synthetic construct
GIDANAVAPAMWDLARAYADVQASRSAILSGSTPSNPSITR

SEQ ID NO: 34 - protein, 17 aa, Artificial Sequence
  Synthetic construct
IQNGNYVSNLAEVTHGR

SEQ ID NO: 35 - protein, 18 aa, Artificial Sequence
  Synthetic construct
IVWNMMLATHTPPANWAK

SEQ ID NO: 36 - protein, 17 aa, Artificial Sequence
  Synthetic construct
KGIDANAVAPAMWDLAR

SEQ ID NO: 37 - protein, 18 aa, Artificial Sequence
  Synthetic construct
KKGIDANAVAPAMWDLAR

SEQ ID NO: 38 - protein, 21 aa, Artificial Sequence
  Synthetic construct
KPAENIPSQEPQPADPADPTR

SEQ ID NO: 39 - protein, 29 aa, Artificial Sequence
  Synthetic construct
KPAENIPSQEPQPADPADPTRAPTLEDLK

SEQ ID NO: 40 - protein, 7 aa, Artificial Sequence
  Synthetic construct
LLGDLFR

SEQ ID NO: 41 - protein, 8 aa, Artificial Sequence
  Synthetic construct
LLGDLFRK

SEQ ID NO: 42 - protein, 5 aa, Artificial Sequence
  Synthetic construct
MPNQK

SEQ ID NO: 43 - protein, 14 aa, Artificial Sequence
  Synthetic construct
MPNQKEIQAHSTAK

SEQ ID NO: 44 - protein, 18 aa, Artificial Sequence
  Synthetic construct
MSNSGSAAAAPSQPSAAK

SEQ ID NO: 45 - protein, 8 aa, Artificial Sequence
  Synthetic construct
QFCMYFAK

SEQ ID NO: 46 - protein, 8 aa, Artificial Sequence
  Synthetic construct
QGLPEDCK

SEQ ID NO: 47 - protein, 32 aa, Artificial Sequence
  Synthetic construct
QGLPEDCKYAGFDFFEGVLSPSALEPADGLIR

SEQ ID NO: 48 - protein, 10 aa, Artificial Sequence
  Synthetic construct
QLYSIDLTPR

SEQ ID NO: 49 - protein, 19 aa, Artificial Sequence
  Synthetic construct
QRIQNGNYVSNLAEVTHGR

SEQ ID NO: 50 - protein, 16 aa, Artificial Sequence
  Synthetic construct
SAILSGSTPSNPSITR

SEQ ID NO: 51 - protein, 17 aa, Artificial Sequence
  Synthetic construct
SNSGSAAAAPSQPSAAK

SEQ ID NO: 52 - protein, 5 aa, Artificial Sequence
  Synthetic construct
TALAK

SEQ ID NO: 53 - protein, 24 aa, Artificial Sequence
  Synthetic construct
YAGFDFFEGVLSPSALEPADGLIR

SEQ ID NO: 54 - protein, 7 aa, Artificial Sequence
  Synthetic construct
SLSLIQT

SEQ ID NO: 55 - protein, 7 aa, Artificial Sequence
  Synthetic construct
EAKGLIR

SEQ ID NO: 56 - protein, 7 aa, Artificial Sequence
  Synthetic construct
TATYLLD

SEQ ID NO: 57 - protein, 7 aa, Artificial Sequence
  Synthetic construct
FHENWPS

SEQ ID NO: 58 - protein, 7 aa, Artificial Sequence
  Synthetic construct
FHEFWPT

SEQ ID NO: 59 - protein, 9 aa, Artificial Sequence
  Synthetic construct
IMDQVPFSV

SEQ ID NO: 60 - protein, 9 aa, Artificial Sequence
  Synthetic construct
GILGFVFTL

SEQ ID NO: 61 - DNA, 12 bp, Artificial Sequence
  Synthetic construct
actagtacgcgt

SEQ ID NO: 62 - DNA, 765 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattcactagtaccacgcgtcaccatcaccatcaccattag

SEQ ID NO: 63 - protein, 254 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFTSTTRHHHHHH

SEQ ID NO: 64 - DNA, 789 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattcactagtggtggcggtctgctgctgactagtaccacgcgtcaccatcaccatcaccattag

SEQ ID NO: 65 - protein, 262 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFTSGGGLLLTSTTRHHHHHH

SEQ ID NO: 66 - DNA, 828 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattcactagttctccgctgaccaaaggtatcctgggtttcgttttcaccctgaccgttccgtctgaaactagtaccacgcgtcaccatcaccatcaccattag

SEQ ID NO: 67 - protein, 275 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFTSSPLTKGILGFVFTLTVPSETSTTRHHHHHH

SEQ ID NO: 68 - DNA, 828 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattcactagttcttctgcgttcaccatcatggaccaggttccgttctctgtttctgtttctcagctgactagtaccacgcgtcaccatcaccatcaccattag

SEQ ID NO: 69 - protein, 275 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFTSSSAFTIMDQVPFSVSVSQLTSTTRHHHHHH

SEQ ID NO: 70 - DNA, 831 bp, Artificial Sequence
  Synthetic construct
atgtcgaactctggttcagccgctgccgctccatctcaaccttctgctgccaagaaacccgctgagaacatcccttcccaagaaccacaacccgcggaccctgctgatcccactagggctcctactcttgaggatctcaaagctatcaattatgtgtcaacaaccacagccgtggcaaccccagcagaaatcaaactactgggcgatctgtttcggaaaaagggtattgatgccaatgctgtggctcctgctatgtgggatctcgctcgtgcttatgcggatgtccaagctagtcgctcagctattctgtctggtagcacaccctccaatccgtccataacacgcacagccttggctaaacaattgtactcaattgacctcactccacggcaattttgtatgtactttgccaagattgtctggaacatgatgcttgccactcacactccacctgccaattgggctaagcagggtctccctgaggattgcaaatatgcaggctttgatttctttgagggtgtcctgtccccatctgctttggagcctgctgatggactcatccgcatgcccaatcagaaagaaattcaagcccactctacagccaaatatggatcactagcacgtcagcgcatccagaatggaaactacgtttccaacttggccgaggtaactcatggtcgtgccggaggagtcaatgcaatgtatgccattgaagcccccccagaattcactagtggtggcggtctgctgctgaaagcgtactctaactgctacccgtacgacgttccggactacactagtaccacgcgtcaccatcaccatcaccattag

SEQ ID NO: 71 - protein, 276 aa, Artificial Sequence
  Synthetic construct
MSNSGSAAAAPSQPSAAKKPAENIPSQEPQPADPADPTRAPTLEDLKAINYVSTTTAVATPAEIKLLGDLFRKKGIDANAVAPAMWDLARAYADVQASRSAILSGSTPSNPSITRTALAKQLYSIDLTPRQFCMYFAKIVWNMMLATHTPPANWAKQGLPEDCKYAGFDFFEGVLSPSALEPADGLIRMPNQKEIQAHSTAKYGSLARQRIQNGNYVSNLAEVTHGRAGGVNAMYAIEAPPEFTSGGGLLLKAYSNCYPYDVPDYTSTTRHHHHHH

SEQ ID NO: 72 - protein, 14 aa, Artificial Sequence
  Synthetic construct
KAYSNCYPYDVPDY

SEQ ID NO: 73 - protein, 19 aa, Artificial Sequence
  Synthetic construct
SPLTKGILGFVFTLTVPSE

SEQ ID NO: 74 - protein, 24 aa, Artificial Sequence
  Synthetic construct
MSLLTEVETPIRNEWGCRCNDSSD

SEQ ID NO: 75 - protein, 24 aa, Artificial Sequence
  Synthetic construct
HSLLTEVETPTRNEWECRCSDSSD

SEQ ID NO: 76 - protein, 24 aa, Artificial Sequence
  Synthetic construct
MSLLTEVETPTRNGWECKCSDSSD

SEQ ID NO: 77 - protein, 24 aa, Artificial Sequence
  Synthetic construct
MSLLTEVETPTRNGWGCRCSDSSD

SEQ ID NO: 78 - protein, 24 aa, Artificial Sequence
  Synthetic construct
MSLLTEVETPTRNEWGCRCSDSSD